(19) United States
(12) Patent Application Publication
(10) Pub. No.: US 2025/0349382 A1
(43) Pub. Date: Nov. 13, 2025

(54) COMPOSITIONS AND METHODS FOR CRISPR-CAS GUIDE RNA DESIGN

(71) Applicants: The Regents of the University of California, Oakland, CA (US); GlaxoSmithKline Intellectual Property Development Limited, Stevenage (GB)

(72) Inventors: Adam Litterman, Oakland, CA (US); Amy Yi-Tyng Chang, Oakland, CA (US); Shawn Shafer, Collegeville, PA (US); Laralynne Przybyla, Oakland, CA (US); Eric D. Chow, Oakland, CA (US)

(21) Appl. No.: 19/195,240

(22) Filed: Apr. 30, 2025

Related U.S. Application Data
(60) Provisional application No. 63/646,514, filed on May 13, 2024.

(51) Int. Cl. G16B 15/30; C12N 9/22; C40B 40/08; G16B 25/10

(52) U.S. Cl. CPC G16B 15/30; C12N 9/222; C40B 40/08; G16B 25/10; C12N 2310/20

(57) ABSTRACT

Methods and compositions are provided for using transcription start site (TSS) profiling to identify alternate promoters that yield better transcription modulation (e.g., knockdown using CRISPRi or activation using CRISPRa). Methods can also include designing CRISPR-Cas guide RNAs to target CRISPRi or CRISPRa complexes to the identified alternative promoters, and methods can also include steps of generating such guide RNAs. Also provided are libraries of guide RNA, methods of modulating expression of a target gene. In some cases, multiple promoters for the same gene are targeted simultaneously.

### CROSS-REFERENCE

This application claims the benefit of U.S. Provisional Patent Application No. 63/646,514 filed May 13, 2024, which application is incorporated herein by reference in its entirety.

### INCORPORATION BY REFERENCE OF SEQUENCE LISTING PROVIDED AS AN XML FILE

A Sequence Listing is provided herewith as a Sequence Listing XML, “UCSF-772WO_SEQ_LISTING.xml” created on Apr. 29, 2025 and having a size of 392,063 bytes. The contents of the Sequence Listing XML are incorporated by reference herein in their entirety.

### I. INTRODUCTION

Bacterial adaptive immune systems employ CRISPRs (clustered regularly interspaced short palindromic repeats) and CRISPR-associated (Cas) proteins for RNA-guided nucleic acid cleavage. CRISPR-Cas effector proteins and their guide RNAs have found use in a variety of applications, including gene editing, nucleic acid detection, and transcription control.

### II. SUMMARY

Current CRISPR-Cas guide RNA libraries for transcription control using, e.g., CRISPRi, target a single transcription start site (TSS) that was identified as the top or second most canonical by a large consortium effort to profile TSS across human cell types about a decade ago (FANTOM5 project by the FANTOM consortium led by RIKEN in Japan). The work described in the experimental examples below led the inventors to the surprising finding that while these guide RNA designs work for most genes in most cell types, approximately 10% of genes in a given cell type use a non-canonical (alternate) promoter or more than one promoter simultaneously, and thus are not knocked down fully using guide RNAs designed by the consortium. Thus, screens (such as genome wide screens) using such constructs will fail to uncover strong phenotypes for these genes even if they are relevant in the biological process under investigation.

Provided are methods and compositions for an alternative approach-using TSS profiling to identify alternate promoters that yield better transcription modulation (e.g., knockdown using CRISPRi or activation using CRISPRa). In some cases, multiple promoters for the same gene are targeted simultaneously. As non-limiting examples, such methods and compositions can be used to: (1) generate libraries of guide RNAs for specific cell types (e.g., guide RNA libraries tailored to a particular cell type/cell line); (2) generate guide RNA construct libraries for use with CRISPR-Cas effector proteins (such as Cas12a, e.g., CRISPRi-dCas12a), where each member of the library encodes an array of guide RNAs (e.g., 5-10, 5-8, 5-7, 6-10, 6-8, 6-7 guide RNAs)—e.g., to target all active promoters for every gene in the human genome, or e.g., to target all sets of matched human gene paralogues simultaneously (e.g., to reveal phenotypes that are obscured by paralogue redundancy); and (3) generate CRISPRa guide RNA libraries for use with CRISPR-Cas effector proteins (such as CRISPRa-dCas12a) that target the TSS and the most active proximal enhancers simultaneously to drive higher levels of gene expression than existing CRISPRa genome wide library designs.

As such, provided are methods for identifying promoters for targeted expression modulation. In some embodiments, a subject method is for identifying one or more target genes for alternative promoter targeting, which can also be referred to as a method of identifying promoters (e.g., alternative/alternate promoters), e.g., for expression modulation. In some cases, a subject method includes a step of designing CRISPR-Cas guide RNAs to target CRISPRi or CRISPRa complexes to alternative promoters of the identified target genes. Such methods can be referred to as, e.g., a method of designing a CRISPR-Cas guide RNA library. In some cases, a subject method includes a step of producing (generating) the designed CRISPR-Cas guide RNAs. Such methods can be referred to as, e.g., a method of producing (or generating) a CRISPR-Cas guide RNA library.

Subject methods can include assessing, for a cell type of interest, using a computer: (i) transcript start site (TSS) expression data, and (ii) genome annotation data, thereby generating quantitative data relating to active promoters and their relative genomic location. Such methods can also include computer processing the quantitative data to identify, for each of a plurality of genes, the most highly utilized promoter that is responsible for at least a threshold percentage of transcription (e.g., 40% or more, 50% or more, or 60% or more of the total transcription across all TSSs assigned to a particular gene), thereby identifying promoters for targeted expression modulation for the cell type of interest. In some cases, at least one of the identified promoters for targeted expression modulation is an alternative promotor and is therefore not a primary promoter as identified by the FANTOM consortium FANTOM5 project (see, e.g., Bertin et al., Sci Data. 2017 Oct. 3:4:170147 as well as Andersson et al., Nature. 2014 Mar. 27; 507 (7493): 455-461; Kawaji et al., Sci Data. 2017 Aug. 29:4:170113; Abugessaisa et al., Methods Mol Biol. 2017; 1611:199-217; Lizio et al., Nucleic Acids Res. 2019 Jan. 8; 47 (D1): D752-D758; Abugessaisa et al., Sci Data. 2017 Aug. 29:4:170107; Noguchi et al., Sci Data. 2017 Aug. 29:4:170112) or as targeted by CRISPRi v3 guide RNA library (see Replogle et al., eLife. 2022 Dec. 28:11:e81856).

In some cases, e.g., when the method is a method of identifying one or more target genes for alternative promoter targeting, a subject method includes computer processing the quantitative data (generated from the assessing) to identify genes for which the most highly utilized promoter is responsible for at least a threshold percentage of transcription (e.g., 40% or more, 50% or more, or 60% or more of the total transcription across all TSSs assigned to a particular gene) and is not the primary promoter of the target gene as identified by the FANTOM5 project or as targeted by CRISPRi v3 guide RNA library.

For any of the subject methods, in some embodiments, the genome annotation data (which can in some cases be analyzed using software, e.g., bedtools) comprises genomic location data for gene annotations and for known CRISPR-Cas guide RNA targets (e.g., CRISPRi v3 guide RNA library). In some cases, the genome annotation data further comprises genomic location data for exon annotations (which also can in some cases, e.g., be analyzed using bedtools software).

In some cases, a subject method includes designing CRISPR-Cas guide RNAs to target CRISPRi or CRISPRa complexes to identified alternative promoters, e.g., alternative promoters of the identified target genes. Such methods can be referred to as, e.g., a method of designing a CRISPR-Cas guide RNA library. In some cases, a subject method includes producing (generating) the designed CRISPR-Cas guide RNAs. Such methods can be referred to as a method of producing (or generating) a CRISPR-Cas guide RNA library.

Also provided are guide RNA libraries, e.g., libraries designed/generated using a subject method. Also provided are methods of modulating expression of a target gene, e.g., in some cases, methods of introducing a subject library into a population of target cells. Reagents, compositions, and kits/systems that find use in practicing the subject methods are provided.

### III. BRIEF DESCRIPTION OF THE DRAWINGS

The following detailed description of embodiments of the invention will be better understood when read in conjunction with the appended drawings. It should be understood that the invention is not limited to the precise arrangements and instrumentalities of the embodiments shown in the drawings.

FIG. 1 illustrates an example workflow for identifying alternative promoter usage in a user-defined cell line of interest.

FIG. 2 provides sequences of the CRISPRi dual guide pairs used in the Perturb-seq K562 experiment. For “protospacer_A” column, from top to bottom is SEQ ID NOs: 1-222. For “protospacer_B” column, from top to bottom is SEQ ID NOs: 223-444.

FIG. 3 provides the results of the Perturb-seq experiment for Group A guide pairs with 4 guides per gene, including % knockdown and p-value data.

FIG. 4 provides the results of the Perturb-seq experiment for Group A guide pairs with 5 guides per gene, including % knockdown and p-value data.

FIG. 5 provides the results of the Perturb-seq experiment for Group B guide pairs, including % knockdown and p-value data.

FIG. 6 depicts NCAM2 expression levels for cells assigned an NCAM2-targeting guide (A1, A2, A3, or A4), labeled “vector-positive” and for negative control cells, labeled “target_negative”. When A1 and A2 guide pairs targeting the canonical NCAM2 promoter as published in the CRISPRi v3 library are used, no significant changes in transcriptional expression are observed. However, when A3 and A4 guide pairs are used to target alternative promoters identified in K562 CAGE data, statistically significant transcriptional knockdown is observed.

FIG. 7 depicts ACIN1 expression levels for cells assigned an ACIN1-targeting guide (B1, B2, or B3) and for negative control cells, labeled “target_negative”. When B1 and B2 guide pairs targeting either the P1 or P2 canonical promoter as published in the CRISPRi v3 library are used, statistically significant, but moderate transcriptional repression is observed. However, when B3 guide pairs are used, where guide pairs are split across the two promoters, statistically significant and more pronounced transcriptional repression is observed.

FIG. 8 depicts a generalized schematic for one example embodiment for identifying an alternative promoter. In this example, Gene X (“Annotated gene”) in a cell type of choice (e.g., K562 cells) has 6 identified peaks relatively nearby—A, B, C, D, E, and F. Peak A: >500 bp from gene annotation, so peak A is eliminated as a candidate alternate TSS for guide RNA targeting. Peak B: <500 bp from gene annotation and >5 kb from CRISPRi v3 library TSS guide RNA (Peak D, see below), so peak B is a candidate alternate TSS for guide RNA targeting. Peak C: <500 bp from gene annotation and >5 kb from CRISPRi v3 library TSS guide RNA (Peak D), so peak C is a candidate alternate TSS for guide RNA targeting. Peak D (has asterisk in figure): TSS to which CRISPRi v3 library TSS guide RNAs are targeted. Peak E: <500 bp from gene annotation, but <5 kb from CRISPRi v3 library TSS guide RNA (Peak D), so peak is eliminated as a candidate alternate TSS for guide RNA targeting. Peak F: <500 bp from gene annotation and >5 kb from CRISPRi v3 library TSS guide RNA (Peak D), so peak F is a candidate alternate TSS for guide targeting. Of the candidate peaks there were not eliminated (Peaks B, C, and F), peak C is the dominant peak, and is therefore selected as the alternate TSS to target with new guide RNA(s).

### IV. DEFINITIONS

A DNA sequence that “encodes” a particular RNA is a DNA nucleic acid sequence that is transcribed into RNA. A DNA polynucleotide may encode an RNA (mRNA) that is translated into protein, and the DNA can therefore be said to encode the protein. A DNA may encode an non-coding RNA (ncRNA), i.e., and RNA that is not translated into protein (e.g. tRNA, rRNA, CRISPR-Cas guide RNA).

A “protein coding sequence” or a sequence that encodes a particular protein or polypeptide, is a nucleic acid sequence that is transcribed into mRNA (in the case of DNA) and is translated (in the case of mRNA) into a polypeptide in vitro or in vivo when placed under the control of appropriate regulatory sequences. The boundaries of the coding sequence are determined by a start codon at the 5′ terminus (N-terminus) and a translation stop nonsense codon at the 3′ terminus (C-terminus). A transcription termination sequence will usually be located 3′ to the coding sequence.

As used herein, a “promoter sequence” is a DNA regulatory region capable of binding RNA polymerase and initiating transcription of a downstream (3′ direction) coding or non-coding sequence. Eukaryotic promoters will often, but not always, contain “TATA” boxes and “CAT” boxes. Various promoters, including inducible promoters, may be used to drive the various vectors of the present invention. The promoter may be a constitutively active promoter, i.e. a promoter is active in the absence externally applied agents (e.g., CMV, EF1a, beta-Actin), or it may be an inducible promoter (e.g., T7 RNA polymerase promoter, heat shock promoter, Tetracycline-regulated promoter, Steroid-regulated promoter, Metal-regulated promoter, doxycycline-regulated promoter, etc). As used herein, an inducible promoter is a promoter whose activity is regulated by a factor that induces expression, e.g., upon the application of an agent to the cell, (e.g. doxycycline), the induced presence of a particular RNA polymerase (e.g., T7 RNA polymerase), and the like. When referring to a nucleic acid encoding an small non-coding RNA (e.g., a CRISPR-Cas guide RNA, an shRNA, a microRNA, an siRNA), the nucleotide sequence encoding the non-coding RNA can be operably linked to a pol III promoter such as a human U6 small nuclear promoter (U6) (Miyagishi et al., Nature Biotechnology 20, 497-500 (2002)), an enhanced U6 promoter (e.g., Xia et al., Nucleic Acids Res. 2003 Sep. 1; 31 (17)), a human H1 promoter (H1), and the like.

The terms “DNA regulatory sequences,” “control elements,” and “regulatory elements,” used interchangeably herein, refer to transcriptional and translational control sequences, such as promoters, enhancers, polyadenylation signals, terminators, protein degradation signals, and the like, that provide for and/or regulate transcription of a non-coding sequence (e.g., a guide RNA) or a coding sequence (e.g., a CRISPRi or CRISPRa fusion protein) and/or regulate translation of an encoded polypeptide.

“Exogenous,” is used herein to refer to something not endogenous to the cell. For example, when an expression vector encoding a CRISPRi or CRISPRa fusion protein is delivered to a cell, the expression vector is exogenous to the cell—the expression vector is an exogenous nucleic acid.

“Recombinant,” as used herein, means that a particular nucleic acid (DNA or RNA) is the product of various combinations of cloning, restriction, polymerase chain reaction (PCR) and/or ligation steps resulting in a construct having a structural coding or non-coding sequence distinguishable from endogenous nucleic acids found in natural systems. DNA sequences encoding polypeptides can be assembled from cDNA fragments or from a series of synthetic oligonucleotides, to provide a synthetic nucleic acid which is capable of being expressed from a recombinant transcriptional unit contained in a cell or in a cell-free transcription and translation system. Genomic DNA comprising the relevant sequences can also be used in the formation of a recombinant gene or transcriptional unit. Sequences of non-translated DNA may be present 5′ or 3′ from the open reading frame, where such sequences do not interfere with manipulation or expression of the coding regions, and may indeed act to modulate production of a desired product by various mechanisms. Alternatively, DNA sequences encoding RNA (e.g., a guide RNA) that is not translated may also be considered recombinant. Thus, e.g., the term “recombinant” polynucleotide or “recombinant” nucleic acid refers to one which is not naturally occurring, e.g., is made by the artificial combination of two otherwise separated segments of sequence through human intervention. This artificial combination is often accomplished by either chemical synthesis means, or by the artificial manipulation of isolated segments of nucleic acids, e.g., by genetic engineering techniques. Such is usually done to replace a codon with a codon encoding the same amino acid, a conservative amino acid, or a non-conservative amino acid. Alternatively, it is performed to join together nucleic acid segments of desired functions to generate a desired combination of functions. This artificial combination is often accomplished by either chemical synthesis means, or by the artificial manipulation of isolated segments of nucleic acids, e.g., by genetic engineering techniques.

A “vector” or “expression vector” is a replicon, such as plasmid, phage, virus, or cosmid, to which another DNA segment, i.e. an “insert”, may be attached so as to bring about the replication and/or expression of the attached segment in a cell.

An “expression cassette” comprises a DNA coding sequence operably linked to a promoter. “Operably linked” refers to a juxtaposition wherein the components so described are in a relationship permitting them to function in their intended manner. For instance, a promoter is operably linked to a coding sequence if the promoter affects its transcription or expression. The coding sequence can also be said to be operably linked to the promoter.

The terms “recombinant expression vector,” or “DNA construct” are used interchangeably herein to refer to a DNA molecule comprising a vector and at least one insert. Recombinant expression vectors are usually generated for the purpose of expressing and/or propagating the insert(s), or for the construction of other recombinant nucleotide sequences. The insert(s) may or may not be operably linked to a promoter sequence and may or may not be operably linked to DNA regulatory sequences.

A cell has been “genetically modified” or “transformed” or “transfected” by exogenous DNA, e.g. a recombinant expression vector, when such DNA has been introduced inside the cell. The presence of the exogenous DNA results in permanent or transient genetic change. The transforming DNA may or may not be integrated (covalently linked) into the genome of the cell. A stably transformed cell is one in which the transforming DNA has become integrated into a chromosome so that it is inherited by daughter cells through chromosome replication. This stability is demonstrated by the ability of the eukaryotic cell to establish cell lines or clones that comprise a population of daughter cells containing the transforming DNA. A “clone” is a population of cells derived from a single cell or common ancestor by mitosis. A “cell line” is a clone of a primary cell that is capable of stable growth in vitro for many generations.

As used herein, a first molecule “specifically binds” or “preferentially binds” or “targets” another molecule if it binds with greater affinity, avidity, more readily, and/or with greater duration than it binds to other substances, e.g., in a sample, in a cell, etc. In some embodiments, a first molecule “specifically binds” or “targets” if it binds to or associates with the target molecule with an affinity or Ka (that is, an association rate constant of a particular binding interaction with units of 1/M) of, for example, greater than or equal to about 105 M−1. In certain embodiments, the first molecule binds with a Ka greater than or equal to about 106 M−1, 107 M−1, 108 M−1, 109 M−1, 1010 M−1, 1011 M−1, 1012 M−1, or 1013 M−1. Alternatively, affinity may be defined as an equilibrium dissociation constant (KD) of a particular binding interaction with units of M (e.g., 10-5 M to 10-13 M, or less). In some aspects, specific binding means the targeting moiety binds to the target molecule with a KD of less than or equal to about 10-5 M, less than or equal to about 10-6 M, less than or equal to about 10-7 M, less than or equal to about 10-8 M, or less than or equal to about 10-9 M, 10-10 M, 10-11 M, or 10-12 M or less. The binding affinity of a first molecule for a target molecule can be readily determined using conventional techniques, e.g., by competitive ELISA (enzyme-linked immunosorbent assay), equilibrium dialysis, by using surface plasmon resonance (SPR) technology (e.g., the BIAcore 2000 or BIAcore T200 instrument, using general procedures outlined by the manufacturer); by radioimmunoassay; or the like.

The term “targets” can also be used to describe complementarity between nucleic acid molecules. As a non-limiting example, a guide RNA that hybridizes to a particular target sequence within a gene of a target DNA (the guide sequence of the guide RNA hybridizes to the target sequence of a target DNA) can be said to “target” that gene. Likewise, the guide RNA can be said to “target” that particular sequence. For example, a guide RNA that targets a particular alternate promoter (e.g., one identified using the subject methods) has a guide sequence that hybridizes to the target DNA such that the CRISPRi or CRISPRa protein it is complexed with modulates transcription from that promoter (i.e., from the targeted promoter). In other words, a guide RNA can be said to target a particular TSS. For example, if multiple guide RNAs are said to target the same particular gene, some may target one particular TSS/promoter of that gene while others may target a different TSS/promoter. Thus guide RNAs can be referred to as targeting a particular gene, and can also be referred to as targeting a particular promoter or TSS.

Suitable methods of genetic modification (also referred to as “transformation”) include viral infection (transduction), transfection, electroporation, particle gun technology, calcium phosphate precipitation, direct microinjection, and the like. The choice of method is generally dependent on the type of cell being transformed and the circumstances under which the transformation is taking place (e.g., in vitro, ex vivo, or in vivo). A general discussion of these methods can be found in Ausubel, et al., Short Protocols in Molecular Biology, 3rd ed., Wiley & Sons, 1995.

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It is noted that, as used herein and in the appended claims, the singular forms “a”, “an”, and “the” include plural referents unless the context clearly dictates otherwise. As such, the articles “a” and “an” are used herein to refer to one or to more than one (i.e., to at least one) of the grammatical object of the article. By way of example, “an element” means one element or more than one element. Thus, for example, reference to “a cell” includes a plurality of such cells and reference to “the polypeptide” includes reference to one or more polypeptides and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible. For example, it is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

While the apparatus and method has or will be described for the sake of grammatical fluidity with functional explanations, it is to be expressly understood that the claims, unless expressly formulated under 35 U.S.C. § 112, are not to be construed as necessarily limited in any way by the construction of “means” or “steps” limitations, but are to be accorded the full scope of the meaning and equivalents of the definition provided by the claims under the judicial doctrine of equivalents, and in the case where the claims are expressly formulated under 35 U.S.C. § 112 are to be accorded full statutory equivalents under 35 U.S.C. § 112.

### V. DETAILED DESCRIPTION

As noted above, provided are methods and compositions for using TSS profiling to identify alternate promoters that yield better transcription modulation (e.g., knockdown using CRISPRi or activation using CRISPRa). As such, provided are methods for identifying promoters for targeted expression modulation. In some embodiments, a subject method is for identifying one or more target genes for alternative promoter targeting, which can also be referred to as a method of identifying promoters (e.g., alternative/alternate promoters), e.g., for expression modulation. In some cases, a subject method includes a step of designing CRISPR-Cas guide RNAs to target CRISPRi or CRISPRa complexes to alternative promoters of the identified target genes. Such methods can be referred to as, e.g., a method of designing a CRISPR-Cas guide RNA library. In some cases, a subject method includes a step of producing (generating) the designed CRISPR-Cas guide RNAs. Such methods can be referred to as, e.g., a method of producing (or generating) a CRISPR-Cas guide RNA library.

In some cases, at least one of the identified promoters for targeted expression modulation is an alternative promotor and is therefore not a primary promoter as identified by the FANTOM consortium FANTOM5 project.

### Compositions and Methods

### FANTOM and CRISPRi v3 Guide RNA Library

As noted above, in some cases, at least one of the identified promoters from a subject method (e.g., for targeted expression modulation) is an alternative promotor and this therefore not a primary promoter as identified by the FANTOM consortium FANTOM5 project (see, e.g., Bertin et al., Sci Data. 2017 Oct. 3:4:170147 as well as Andersson et al., Nature. 2014 Mar. 27; 507 (7493): 455-461; Kawaji et al., Sci Data. 2017 Aug. 29:4:170113; Abugessaisa et al., Methods Mol Biol. 2017; 1611:199-217; Lizio et al., Nucleic Acids Res. 2019 Jan. 8; 47 (D1): D752-D758; Abugessaisa et al., Sci Data. 2017 Aug. 29:4:170107; Noguchi et al., Sci Data. 2017 Aug. 29:4:170112) or as targeted by CRISPRi v3 guide RNA library (see Replogle et al., eLife. 2022 Dec. 28:11:e81856).

See, e.g., Noguchi et al., Sci Data. 2017 Aug. 29:4:170112. Since the completion of the human genome sequencing, the role of individual bases has been a central question. An international collaborative effort, FANTOM (Functional ANnoTation Of Mammalian Genome), delineated a complex landscape of transcribed RNAs (transcriptome) and their regulations. The initial key technology driving the project was to make full-length cDNA clones, representing complete primary structure of transcribed RNA molecules. Sequencing of the full-length cDNA clones uncovered unexpected number of long non-coding RNAs as well as protein coding genes. The CAGE (Cap Analysis Gene Expression) protocol, in combination with high-throughput sequencing, was developed to monitor frequencies of transcription initiation by determining 5′-end of capped RNAs. The technology was devised to uncover complexity of the transcriptome and elucidate transcriptional regulatory networks by focusing on promoter elements.

In the fifth round of the FANTOM projects, FANTOM5, the challenge was to capture the transcriptome of many varieties of cell states as possible, to understand the implication of each genomic bases in different contexts. In the first phase of the FANTOM5 project, cells were targeted in steady state, called ‘snapshot’ samples. The central focus was on human primary cells, while cell lines, tissues and mouse samples were chosen to cover cells inaccessible as isolated human primary samples. The resulting data provided an atlas of promoter and enhancer activities in wide range of cell states, which is a baseline of understanding complex transcriptional regulation. In the second phase, the focus was on transitions of cell states by monitoring ‘time course’ samples, such as activations, differentiations, and developments at sequential time points. The monitored activities of promoters and enhancers demonstrated that enhancer activities is the earliest event during dynamic changes of transcriptome. These data sets are available and are being utilized in studies inside and outside of the FANTOM5.

As used herein, terms such as “CRISPRi v3 guides”, “CRISPRi v3 guide RNA library”, “CRISPRi v3 guide RNAs”, and “CRISPRi v3 guide RNA”, etc. refer to the CRISPRi guide RNA library generated by Replogle et al. (eLife. 2022 Dec. 28:11:e81856). This guide RNA library was designed based on consensus TSS information across many different cell types/cell lines as provided by CAGE data from FANTOM5. More specifically, guide RNAs of the CRISPRi v3 guide RNA library were designed to target the “primary promoter” of each targeted gene. The primary promoter can also be referred to herein as the “canonical promoter.” The primary promoter was determined based on consensus TSS information across many different cell types, i.e., the primary promoter is the highest ranking (top-used) promoter when analyzing TSS data across many different cell types. As such, for genes that use an “alternate promoter” (also referred to herein as “alternative promoter”) (a promotor that is not the primary promoter) in some cell types but not others, the alternate promoters were not targeted by the CRISPRi v3 guide RNA library. To the contrary, the methods described herein are for facilitating guide RNA design in a particular cell type of interest, and therefore the guide RNA libraries designed using the subject methods include guide RNAs that target alternate promoters, i.e., promoters that were not targeted by the CRISPRi v3 guide RNA library.

### TSS Peaks

“Transcript start site (TSS) expression data” refers to data related to the location of transcriptional start sites in a given target genomic DNA (e.g., genome of a cell type of interest). As would be known to one of ordinary skill in the art, an example of such data is in the form of sequencing reads, e.g., performed with CAGE analysis. Many methods are available for producing/providing TSS expression data, and any convenient method can be used. Examples include, but are not necessarily limited to: oligo-capping methods such as 5′ Serial Analysis of Gene Expression (5′ SAGE), TSS-seq, Paired-End Analysis of TSSs (PEAT), CapSeq, TL-seq, Transcript IsoForm sequencing (TIF-seq), and Simultaneous Mapping of RNA Ends by sequencing (SMORE-seq); cap-trapping methods such as Cap Analysis of Gene Expression (CAGE) and Multiplexed Affinity Purification of Capped RNA (MAPCap); and template-switching reverse transcription methods such as template-switching reverse transcription (TSRT), nanocage, CAGEscan, NanoCAGE-XL, RNA Annotation and Mapping of Promoters for the Analysis of Gene Expression (RAMPAGE), Tn5Prime, low-input Parallel Analysis of RNA Ends (nanoPARE), Survey of TRanscription Initiation and Promoter Elements with high-throughput sequencing (STRIPE-seq). In some cases, methods that capture TSSs from nascent transcripts can be used, e.g., Global Run-On sequencing (GRO-seq) and Precision Run-On sequencing (PRO-seq).

In some embodiments, TSS expression data can be provided in the form of CAGE data (e.g., a CAGE bam file). As would be understood by one of ordinary skill in the art, multiple forms of CAGE are available, and any convenient method can be used. Examples of modified versions of the original CAGE include, but are not necessarily limited to: DeepCAGE, HeliScopeCAGE, no-amplification non-tagging CAGE for Illumina sequencers (nAnT-iCAGE), Super-Low-Input Carrier CAGE (SLIC-CAGE), and C1 CAGE. See, e.g., Policastro et al., Cell Rep Methods. 2021 Sep. 27; 1 (5): 100081; Kouno et al., Nat Commun. 2019 Jan. 21; 10 (1): 360; Georgakilas et al., Sci Rep 10, 877 (2020); and Seki et al., Nucleic Acids Research, Volume 52, Issue 2, 25 Jan. 2024, Page e7; as well as U.S. Pat. No. 11,312,991; and US patent publication No US20210164020, which are incorporated by reference herein for disclosures relating to TSS mapping.

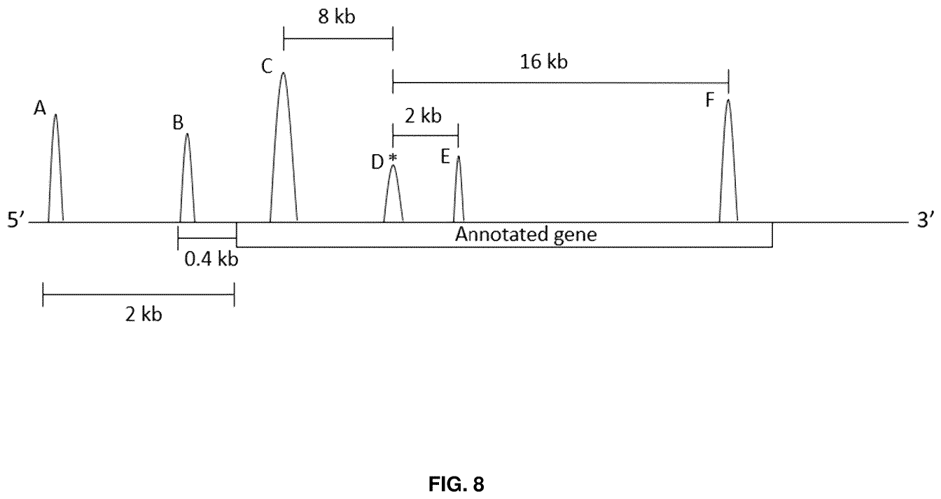

When sequence reads (e.g., from CAGE analysis) accumulate at a particular location in the genome, they can appear as a ‘peak’ when plotted as a histogram along the genome (i.e., the x-axis is the location of the genome). The peaks can represent TSSs. If multiple peaks are present near/within a given gene annotation, than those peaks represent multiple different TSSs for that gene- and are therefore possible candidates for guide RNA design (e.g., for CRISPRi or CRISPRa). See, e.g., FIG. 8. As such, the methods disclosed herein can provide a method for determining which peak(s) should be used for targeting guide RNAs for applications such as CRISPRi or CRISPRa. In some cases, the TSS expression data includes total read counts per expression peak.

Readily available software, such as bedtools and MACS2, can be used to sort through/extract peak information. This peak information can be used in combination with genome annotation data to generate quantitative data relating to active promoters and their relative genomic location (e.g., location relative to gene annotations, exon annotations, and in some cases previously designed guide RNAs). As such, in some embodiments a subject method includes assessing, for a cell type of interest, using a computer: TSS expression data and genome annotation data to generate quantitative data relating to active promoters and their relative genomic location. In some cases, the quantitative data (of a subject method) relating to active promoters and their relative genomic location includes expression peak data and the closest gene annotation, the closest exon annotation, and/or the closest CRISPR-Cas guide RNA target annotation (e.g., from CRISPRi v3 guide RNA library) for each expression peak.

This ‘assessing’ can be performed using any convenient data set and software tools. For example, in some embodiments, a subject method includes using genome sequence information, e.g., human genome information such as from a genome assembly, e.g., hg38 (e.g., in some cases provided via gtf file), to make a determination as to where the peaks are located relative to that genome sequence. Available software tools (e.g., bedtools) can be used, e.g., to assign the closest gene annotation and/or exon annotation to each of the TSS peaks. In some cases, multiple TSS peaks will be assigned to the same gene annotation and/or exon annotation. These peaks can then be considered candidates for guide RNA targeting (in some cases subject to a filtering step, see below).

Likewise, as part of ‘assessing’, information such as guide sequence information from previously designed guide RNAs (e.g., for ‘Horlbeck guide RNAs”: see Horlbeck et al., Elife. 2016 Sep. 23:5:e19760; and for “CRISPRi v3 guide RNAs”/“CRISPRi v3 guide RNA library”: see Replogle et al., eLife. 2022 Dec. 28:11:e81856), in some cases provided in the form of a bed file, can be used to determine which of the TSS peaks has been targeted by a previously designed guide RNA library.

### Computer Processing and Filtering

In some embodiments, computer processing is used to identify, for each of a plurality of genes, the most highly utilized promoter that is responsible for at least a threshold percentage of transcription (e.g., 40% or more, 50% or more, or 60% or more of the total transcription across all TSSs assigned to a particular gene). For methods of identifying promoters for targeted expression modulation, such an identification can result in the identification of promoters for targeted expression modulation for the cell type of interest. In some cases, the threshold percentage of transcription (% relative to the total across all TSSs assigned to a particular gene) is 40%. In some cases, the threshold percentage is 45%. In some cases, the threshold percentage is 50%. In some cases, the threshold percentage is 55%. In some cases, the threshold percentage is 60%.

For other related methods, e.g., methods of identifying one or more target genes for alternate promoter targeting, the computer processing can be used to identify genes for which the most highly utilized promoter is responsible for at least a threshold percentage of transcription (e.g., 40% or more, 50% or more, or 60% or more of the total transcription across all TSSs assigned to a particular gene) and is not the primary promoter of the target gene as identified by the FANTOM5 project or as targeted by CRISPRi v3 guide RNA library. Such an identification can result in the identification of target genes for alternate promoter targeting. In some cases, the threshold percentage of transcription (% relative to the total across all TSSs assigned to a particular gene) is 40%. In some cases, the threshold percentage is 45%. In some cases, the threshold percentage is 50%. In some cases, the threshold percentage is 55%. In some cases, the threshold percentage is 60%.

For any of the subject methods, in some cases, the most highly utilized promoter is at least a threshold distance away from the closest known CRISPR-Cas guide RNA target (e.g., the closest known CRISPR-Cas guide RNA from the CRISPRi v3 guide RNA library). In some cases, the threshold distance is 3 kilobases (kb). As such, in some cases, the most highly utilized promoter is 3 kb or more (e.g., 4 kb or more, 5 kb or more) from the closest the closest known CRISPR-Cas guide RNA target (e.g., the closest known CRISPR-Cas guide RNA from the CRISPRi v3 guide RNA library). In some cases, the threshold distance is 4 kilobases (kb). As such, in some cases, the most highly utilized promoter is 4 kb or more (e.g., 5 kb or more) from the closest the closest known CRISPR-Cas guide RNA target (e.g., the closest known CRISPR-Cas guide RNA from the CRISPRi v3 guide RNA library). In some cases, the threshold distance is 5 kilobases (kb). As such, in some cases, the most highly utilized promoter is 5 kb or more from the closest the closest known CRISPR-Cas guide RNA target (e.g., the closest known CRISPR-Cas guide RNA from the CRISPRi v3 guide RNA library).

In some cases, TSS peaks are removed from consideration for CRISPR-Cas guide RNA targeting by applying a particular criteria. In other words, a subject method can include a filtering step. In some cases, the computer processing includes removing (from consideration) expression peaks for which the closest gene annotation is greater than 500 base pairs (bp) away. Such a criteria ensures that only peaks within 500 bp of an annotated gene and/or exon are considered for CRISPR-Cas guide RNA targeting. In some cases, expression peaks that are within 10% of the 3′ end of a gene are removed from consideration for CRISPR-Cas guide RNA targeting.

In some embodiments, the computer processing includes, after filtering, ranking TSS expression peaks for each gene (i.e., on a per-gene basis) based on the ratio of expression for each TSS expression peak (e.g., as can be measured by read counts) to the total expression (e.g., the total read counts) across all expression peaks for that given gene. In other words, ranking can be based on the percentage of reads for each expression peak (reads per peak as a percentage of the total reads across all peaks for the gene). In yet other words: the computer processing can include: (i) determining for each peak, the fraction (percentage) of that given gene's transcription for which the TSS peek is responsible, and (ii) ranking the expression peaks based on their determined fraction of expression for which they are responsible. In some cases, such a ranking includes ranking the primary promoter of the target gene as identified by the FANTOM5 project or as targeted by CRISPRi v3 guide RNA library. Such a ranking would identify whether any of the identified alternate promoters are ranked higher than (i.e., are a stronger promoter than) the primary promoter in the particular cell type of interest that is under investigation. In some cases, ranking ignores the primary promoter of the target gene as identified by the FANTOM5 project or as targeted by CRISPRi v3 guide RNA library (i.e., the peak corresponding to the primary promoter can be removed from consideration prior to ranking the remaining peaks). Such a ranking would identify the strongest alternate promoter, regardless of whether it was stronger than the primary promoter.

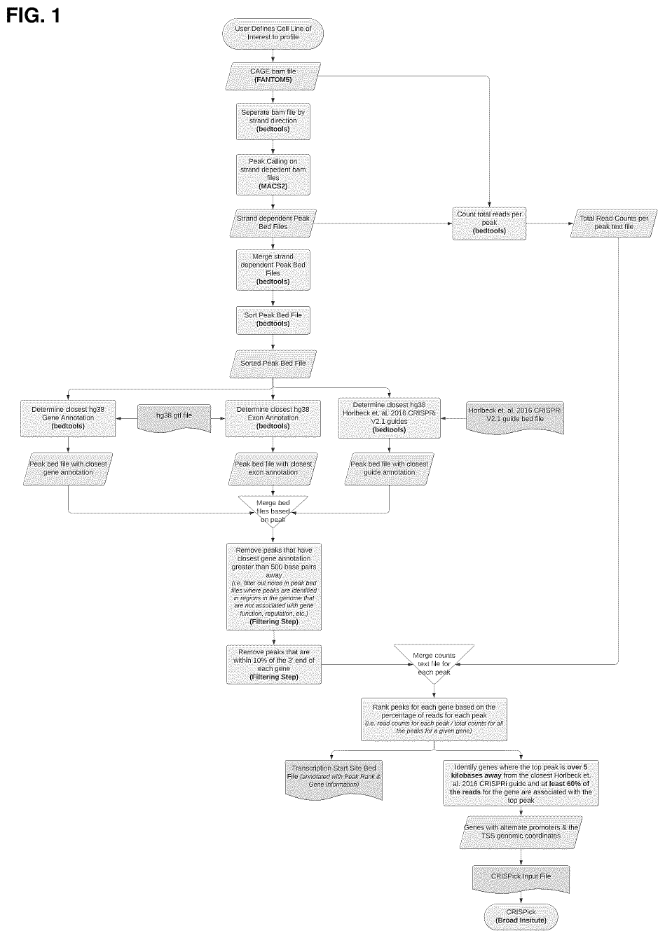

For an example of one possible embodiment of the assessing and computer processing steps, refer to FIG. 1.

In some embodiments, all or a portion of the assessing and/or computer processing steps is performed using a machine learning algorithm. Machine learning algorithms may be unsupervised learning algorithms. Examples of unsupervised learning algorithms may include two-cluster Gaussian mixture models, artificial neural network, Data clustering, Expectation-maximization algorithm, Self-organizing map, Radial basis function network, Vector Quantization, Generative topographic map, Information bottleneck method, and IBSEAD. Unsupervised learning may also comprise association rule learning algorithms such as Apriori algorithm, Eclat algorithm and FP-growth algorithm. Hierarchical clustering, such as Single-linkage clustering and Conceptual clustering, may also be used. Alternatively, unsupervised learning may comprise partitional clustering such as K-means algorithm and Fuzzy clustering. In some embodiments, the machine learning algorithm is a two-cluster Gaussian mixture model (GMM).

The machine learning algorithms may be supervised learning algorithms. Examples of supervised learning algorithms may include Average One-Dependence Estimators (AODE), Artificial neural network (e.g., Backpropagation), Bayesian statistics (e.g., Naive Bayes classifier, Bayesian network, Bayesian knowledge base), Case-based reasoning, Decision trees, Inductive logic programming, Gaussian process regression, Group method of data handling (GMDH), Learning Automata, Learning Vector Quantization, Minimum message length (decision trees, decision graphs, etc.), Lazy learning, Instance-based learning Nearest Neighbor Algorithm, Analogical modeling, Probably approximately correct learning (PAC) learning, Ripple down rules, a knowledge acquisition methodology, Symbolic machine learning algorithms, Subsymbolic machine learning algorithms, Support vector machines, Random Forests, Ensembles of classifiers, Bootstrap aggregating (bagging), and Boosting. Supervised learning may comprise ordinal classification such as regression analysis and Information fuzzy networks (IFN). Alternatively, supervised learning methods may comprise statistical classification, such as AODE, Linear classifiers (e.g., Fisher's linear discriminant, Logistic regression, Naive Bayes classifier, Perceptron, and Support vector machine), quadratic classifiers, k-nearest neighbor, gradient boosted trees, Decision trees (e.g., C4.5, Random forests), Bayesian networks, and Hidden Markov models.

In some instances, the machine learning algorithms comprise a reinforcement learning algorithm. Examples of reinforcement learning algorithms include, but are not limited to, temporal difference learning, Q-learning and Learning Automata. Alternatively, the machine learning algorithm may comprise Data Pre-processing. The machine learning algorithm may be trained on a training dataset or datasets. The datasets may contain TSS peak information, gene annotation information, exon annotation information, guide RNA sequences from CRISPRi v3 guide RNA library, and/or selected TSS peaks that are known to be (or not to be) a suitable/desirable alternative promoter.

### Designing and Generating CRISPR-Cas Guide RNAs

In some cases, a subject method includes designing CRISPR-Cas guide RNAs to target CRISPRi or CRISPRa complexes to identified alternate promoters, e.g., alternate promoters of the identified target genes. Such methods can in some cases be referred to as, e.g., a method of designing a CRISPR-Cas guide RNA library. Once target regions are selected (e.g., alternate promoters are selected for targeting), designing guide RNAs to target those regions can be accomplished using any convenient method and many such methods will be known to one of ordinary skill in the art. For example, in some cases a software program such as CRISPick (a CRISPR guide prediction algorithm) can be used. In some cases, a subject method includes producing (generating) the designed CRISPR-Cas guide RNAs. Such methods can in some cases be referred to as a method of producing (or generating) a CRISPR-Cas guide RNA library.

In some embodiments, multiple TSS for the same gene can be targeted by the CRISPR-Cas guide RNAs. For example, in some cases, the primary promoter is targeted in addition to the highest ranked alternate promoter. In such cases, guide RNAs can be designed to target both peaks (primary and highest ranked alternative).

The CRISPR-Cas guide RNA can be any convenient type of guide RNA. For example, in some cases the guide RAN is a Cas9 guide RNA. As another example, in some cases, the guide RNA is a Cas12a guide RNA.

Guide RNAs can be produced by any convenient method (e.g., in vitro transcription, chemical synthesis, and the like). In some cases, the guide RNAs can be designed to be expressed from an expression vector and/or generated by encoding them on nucleic acid (e.g., an expression vector)—in which case nucleic acids encoding the guide RNAs can be introduced into a cells, and the cells produce the guide RNAs via transcription.

As would be understood by one of ordinary skill in the art, in some cases (e.g., when designing and/or producing a large library) it can be advantageous to encode more than one guide RNA on a single expression vector (e.g., multiple guide RNAs encoded by the same plasmid). In some such cases, the sequences encoding the guide RNAs can be arranged so that the guide RNAs are expressed as part of a guide RNA array-which can then be processed in the cell into separate guide RNAs. In some cases, a nucleic acid (e.g., an expression vector) encodes 2 or more guide RNAs (e.g., 3 or more, 4 or more, 5 or more, 6 or more, 7 or more, 8 or more, 9 or more, or 10 or more, e.g., 2, 3, 4, 5, 6, 7, 8, 9, 10, 11, or 12). In some cases, a nucleic acid (e.g., an expression vector) encodes 4 or more guide RNAs (e.g., Cas12a guide RNAs). In some cases, a nucleic acid (e.g., an expression vector) encodes 4-12 guide RNAs (e.g., 4-10, 4-8, 4-7, 4-6, 4-5, 5-12, 5-10, 5-8, 5-7, 5-6, 6-12, 6-10, 6-8, or 6-7), e.g., Cas12a guide RNAs. In some cases, a nucleic acid (e.g., an expression vector) encodes 5-10 Cas12a guide RNAs. In cases, e.g., when using Cas12a (e.g., a dCas12a CRISPRi protein) and Cas12a guide RNAs, the guide RNAs are designed to target more then one peak for each gene—in some cases 2 peaks, 3 peaks, 4 peaks, or even all TSS peaks associated with each gene (e.g., within 500 bp of each annotated gene).

When referring to a guide RNA or a guide RNA library, it is to be understood that guide RNAs can be provided as an RNA or as a DNA encoding the RNA. Thus, disclosure about a guide RNA library or library of guide RNAs, should also be understood to be disclosure of a library of nucleic acids (e.g., expression vectors) encoding the guide RNAs.

Provided are guide RNA libraries, e.g., libraries designed/generated using a subject method. As such, in some cases, a subject guide RNA library includes a plurality of CRISPR-Cas guide RNAs or nucleic acids encoding the plurality of CRISPR-Cas guide RNAs. In some cases, the CRISPR-Cas guide RNAs are targeted to the most highly utilized promoters identified using the subject methods (e.g., in some cases the most highly utilized alternate promoters). In some cases, a guide RNA library comprises at least one CRISPR-Cas guide RNA targeted to an alternate promoter (a i.e., a promoter which is not a primary promoter identified by the FANTOM5 project or as targeted by CRISPRi v3 guide RNA library). In some cases, a guide RNA library does not include CRISPR-Cas guide RNAs that are targeted to promoters identified by the FANTOM5 project or as targeted by CRISPRi v3 guide RNA library. In some cases, a guide RNA library includes, for each target gene: (i) a CRISPR-Cas guide RNA targeted to an alternate promoter; and (ii) a CRISPR-Cas guide RNA targeted to a primary promoter identified by the FANTOM5 project or as targeted by CRISPRi v3 guide RNA library. Such libraries therefore target at least two promoters per targeted gene.

A guide RNA library can be any convenient size. In some cases, a guide RNA library targets 100-35,000 genes (e.g., 100-30,000, 100-25,000, 100-20,000, 100-15,000, 500-35,000, 500-30,000, 500-25,000, 500-20,000, 500-15,000, 1,000-35,000, 1,000-30,000, 1,000-25,000, 1,000-20,000, 1,000-15,000, 5,000-35,000, 5,000-30,000, 5,000-25,000, 5,000-20,000, 5,000-15,000, 10,000-35,000, 10,000-30,000, 10,000-25,000, 10,000-20,000, or 10,000-15,000). In some cases, a guide RNA library is a genome-wide library. In some cases, a guide RNA library targets 10,000-30,000 genes. For any of the above cases, in some cases,

In some cases, more than one guide RNA (e.g., 2, 3, or 4 guide RNAs) is targeted per selected promoter (per selected TSS peak). As an illustrative example, for a given selected alternative TSS peak, in some cases one guide RNA is targeted to that peak, and in other cases 2, 3, or 4 different guide RNAs are targeted to the peak. The same is true for a primary promoter. In embodiments where a primary promoter is targeted in addition to an alternate promoter—in some cases, one guide RNA is targeted to the primary promoter and in other cases, 2, 3, or 4 different guide RNAs are targeted to the primary promoter.

Thus, the number of different guide RNAs in a give library will not necessarily match with the number of genes targeted. As an illustrative example, if a given library targets 5,000 genes, 10,000 guide RNAs will be needed at minimum if both the primary promoter and an alternate promoter are targeted for each gene. Likewise, 10,000 guide RNAs would be needed if only one alternate promoter is selected per gene (i.e., no primary promoters are targeted), but for each gene 2 guide RNAs are used per promoter. As another illustrative example, if 5,000 genes are targeted, 3 promoters per gene are targeted, and 2 guide RNAs are targeted to each selected promoter, then a minimum of 30,000 guide RNAs would be needed.

### Cell Type of Interest/Target Cells

As discussed elsewhere herein, previous guide RNAs have been designed based on consensus TSS information across different cell types, e.g., as provided by CAGE data from FANTOM5. To the contrary, the methods described herein are for facilitating guide RNA design in a particular cell type of interest. The cell type of interest can be any type of cell. Examples include, but are not limited to: a stem cell, e.g. an embryonic stem (ES) cell, an induced pluripotent stem (iPS) cell, a germ cell (e.g., an oocyte, a sperm, an oogonia, a spermatogonia, etc.), an adult stem cell, a somatic cell, e.g. a fibroblast, a hematopoietic cell, a neuron, a muscle cell, a bone cell, a hepatocyte, a pancreatic cell. For example, suitable cells include, but are not limited to: human embryonic stem cells, fetal cardiomyocytes, myofibroblasts, mesenchymal stem cells, cardiomyocytes, adipocytes, totipotent cells, pluripotent cells, blood stem cells, myoblasts, adult stem cells, bone marrow cells, mesenchymal cells, embryonic stem cells, parenchymal cells, epithelial cells, endothelial cells, mesothelial cells, fibroblasts, osteoblasts, chondrocytes, exogenous cells, endogenous cells, stem cells, hematopoietic stem cells, bone-marrow derived progenitor cells, myocardial cells, skeletal cells, fetal cells, undifferentiated cells, multi-potent progenitor cells, unipotent progenitor cells, monocytes, cardiac myoblasts, skeletal myoblasts, macrophages, capillary endothelial cells, xenogeneic cells, allogeneic cells, and post-natal stem cells. Suitable cells include a cancer cell, a hematopoietic stem cell, a lung cell, a neuron, an astrocyte, an islet cell, a kidney cell, an adipocyte, a hepatocyte, an endothelial cell, a muscle cell, a cardiomyocyte, a retinal cell, a tissue-resident stem cell, an immune cell, a monocyte, a macrophage, a B cell, and a T cell. In some cases, the cell is an immune cell, a neuron, an epithelial cell, and endothelial cell, or a stem cell. In some cases, the immune cell is a T cell, a B cell, a monocyte, a natural killer cell, a dendritic cell, or a macrophage. In some cases, the immune cell is a cytotoxic T cell. In some cases, the immune cell is a helper T cell. In some cases, the immune cell is a regulatory T cell (Treg).

Cells may be from established cell lines or they may be primary cells, where “primary cells”, “primary cell lines”, and “primary cultures” are used interchangeably herein to refer to cells and cells cultures that have been derived from a subject and allowed to grow in vitro for a limited number of passages, i.e. splittings, of the culture. For example, primary cultures are cultures that may have been passaged 0 times, 1 time, 2 times, 4 times, 5 times, 10 times, or 15 times, but not enough times go through the crisis stage. Typically, the primary cell lines are maintained for fewer than 10 passages in vitro. Target cells can be unicellular organisms and/or can be grown in culture. If the cells are primary cells, they may be harvest from an individual by any convenient method. For example, leukocytes may be conveniently harvested by apheresis, leukocytapheresis, density gradient separation, etc., while cells from tissues such as skin, muscle, bone marrow, spleen, liver, pancreas, lung, intestine, stomach, etc. can be conveniently harvested by biopsy.

In some cases, the cell type of interest is a mammalian cell. In some cases, the cell type of interest is a human cell. In some cases, the cell type of interest is a non-human primate cell. A cell type of interest can also be a particular cell line, such as an immortalized cell line. Examples of suitable human cell lines of interest include, but are not limited to: NCI-H295R, 5637, HT-1376, J82, SW 780, T24T24-Luc-Neo, MG-6, Saos-2, SJSA-1, SW 1353, D54-Luc, DBTRG-05M, GGli36-DsRed-R-Luc (rescued), LN-18, LN-229, LN-827 LucNeo, M059K, SF-295, SF-539, SF-767, SNB-19, U-251, U-251-Luc-mCh-Puro, U-87 MG, U-87 MG-Luc, HeL, aKB, C2BBe1, Caco-2, COLO 205, COLO 205-Luc #2, DLD-1, HCC2998, HCT-116, HCT-116-Luc, HCT-15, HCT-8, HT-29, HT-29-Luc, LoVo, LS 174T, LS411N, NCI-H508, SW-480, SW-620, KLE, KLE-Luc-mCh-Puro, A-431, HEKn, HEKn-2, OE33, A-673, RD-ES, HT-1080, GIST-T1, NCI-N87, NUGC-4, MKN-45, NCI-N87, SNU-5, CAL 27, FaDu, KARPAS 299, HL-60, EOL-1, Kasumi-1, Kasumi-3, Kasumi-3-Luc-mCh-Puro, KG-1-Luc-mCh-Puro, MOLM-13, MV-4-11, MV-4-11-Luc-mCh-Puro, NOMO-1, THP-1, NALM6, NALM6-Luc-MCh-Puro, Reh, Reh Luc-Neo, RS4; 11, K-562, K-562-Luc2, HEL, HEL 92.1.7, HEL 92.1.7-Luc-Neo, HEL-Luc-Neo, ARH-77, CCRF-CEM, DND-41-Luc-mCh-Puro, Jurkat, Jurkat-Clone E6-1, MOLT-4, MOLT-4-Luc-MCh-Puro, SW 872, Hep 3B2.1-7, Hep G2, Calu-6, HCC2935, NCI-H1703, NCI-H1703-Luc-mCh-Puro, NCI-H2030, NCI-H2110, NCI-H2122, NCI-H3122, NCI-H322, MNCI-H82, A549, A549-Luc-C8, Calu-1, Calu-3, HCC827, HCC827-Luc-mCh-Puro, NCI-H125, NCI-H125-Luc, NCI-H1299, NCI-H1650, NCI-H1975, NCI-H1975-Luc, NCI-H23, NCI-H292, NCI-H441, NCI-H460, NCI-H460-Luc2, NCI-H522, PC-9, PC-9-Luc-mCh-puro, DMS 114, NCI-H446, NCI-H69, SHP-77, EBC-1, SK-MES-1, DB, DBM2, RL, Farage, B-JAB, Daudi, Daudi-Luc-mCh-Puro, NAMALWA, Raji, Raji-Luc, Ramos, Ramos-Luc, HuT 78, HT, SU-DHL-6, SU-DHL-6-Luc-mCh-Puro, GRANTA-519, OCI-Ly1 LN, OCI-Ly1 R10-Luc-mCh-Puro, OCI-Ly1 R7-Luc-mCh-Puro, OCI-Ly19-Luc-Neo, OCI-Ly3-Luc-mCh-Puro, OCI-Ly7-Luc-Neo, Pfeiffer, SU-DHL-10, SU-DHL-10-LN-High, SU-DHL-16, SU-DHL-4-Luc-mCh-Puro, SU-DHL-8, TMD8, Toledo-Luc-Neo, WSU-DLCL2, WSU-FSCCL, NK-92 MI, KARPAS 299, HCC1395, HCC1806, AU-565, BT-20, BT-474, HCC70, Hs 578Bst, Hs 578T, MCF 10A, MCF-7, MCF7-Luc-mCh-Puro, MDA-MB-231, MDA-MB-231-2LMP, MDA-MB-231-D3H2LN, MDA-MB-231-Luc-D3H1, MDA-MB-231-Luc-D3H2LN, MDA-MB-361, MDA-MB-453, MDA-MB-468, MX-1-Luc, SK-BR-3, T47D, ZR-75-1, A2058, A375, COLO 829, G-361, LOX-IMVI, M14, MDA-MB-435S, OCM-1, OCM-1-Luc-mCh-Puro, SK-MEL-5, SK-MEL-28, SK-MEL-28-Luc-mCh-Puro, UACC-62, WM-115, WM-266-4, JJN-3-Luc, KMS-11, KMS-26, KMS-34, MM.1S (pMMP-Luc-Neo), NCI-H929, NCI-H929-Luc-mCh-Puro, OPM-2, RPMI 8226, U266B1, SK-N-AS, SK-N-FI, SK-N-SH, MKL-1, Hs 895.Sk, MRC-5, A2780, A2780-Luc, IGROV1, IGROV1-Luc-Mch-Puro, NIH: OVCAR-3, NIH: OVCAR-3-Luc-mCh-Puro, OV-90, OVCAR-4, OVCAR-5, OVCAR-5-Luc-mCh-Puro, OVCAR-8, OVCAR-8-Luc-mCh-Puro, PA-1, SK-OV-3 (Subcutaneous), SK-OV-3-Luc-D3 (Intraperitoneal), AsPC-1, Bx-PC-3, BxPC-3-Luc2, Capan-1, Capan-2, KP4, MIA PaCa-2, MIA PaCa-2-Luc, PANC-1, PANC-1-Luc, SU-86.86, SW 1990, Detroit 562, BeWo, 22Rv1, CWR-22-R, DU 145, DU 145-Luc, LnCap clone FGC, PC-3, PC-3-Luc, PC-3M-Luc-C6, VCaP, 293, 293-Luc-mCh-Puro, 293T, 769-P, 786-O, 786-O-Luc-Neo (rescued), A-498, ACHN, Caki-1, TK-10, RPTEC, MB-1, TT, SK-LMS-1.

In some cases, the cell type of interest (i.e., the target cell or population of cells) is a mouse cell, a non-human primate cell, or a human cell. In some cases, the cell type of interest (i.e., the target cell or population of cells) is an immortalized cell line. In some cases, the immortalized cell line is an immortalized mouse cell line, an immortalized non-human primate cell line, or an immortalized human cell line.

### CRISPR-Cas Proteins

Approaches for designing CRISPR-Cas guide RNAs (also simply referred to herein as “guide RNAs”) once a target has been selected, and using CRISPR-Cas systems to increase expression or decrease expression of a target gene (e.g., via CRISPRa or CRISPRi, respectively) or to edit a target gene (e.g., induce a mutation in a target gene) are known in the art and any convenient system can be used. For example, when using CRISPRa or CRISPRi to modulate expression of a target gene, a guide RNA is used that hybridizes at or near a transcription start site to inhibit (CRISPRi) or activate (CRISPRa) expression of the target gene. For example, like other CRISPR approaches, CRISPRi has been paired with large-scale sgRNA libraries to conduct systematic genetic screens. Such screens have been deployed to identify essential protein-coding and non-coding genes (Gilbert et al., 2014; Haswell et al., 2021; Horlbeck et al., 2016a; Liu et al., 2017; Raffeiner et al., 2020), to map the targets of regulatory elements (Fulco et al., 2019; Fulco et al., 2016; Gasperini et al., 2019; Kearns et al., 2015; Klann et al., 2017; Thakore et al., 2015), to identify regulators of cellular signaling and metabolism (Coukos et al., 2021; Liang et al., 2020; Luteijn et al., 2019; Semesta et al., 2020), to uncover stress response pathways in stem cell-derived neurons (Tian et al., 2021; Tian et al., 2019), to uncover regulators of disease-associated states in microglia and astrocytes (Dräger et al., 2022; Leng et al., 2022), to decode regulators of cytokine production in primary human T-cells (Schmidt et al., 2022), to define mechanisms of action of bioactive small molecules (Jost et al., 2017; Morgens et al., 2019; le Sage et al., 2017), to identify synthetic-lethal genetic interactions in cancer cells (Du et al., 2017; Horlbeck et al., 2018), and to identify genetic determinants of complex transcriptional responses using RNA-seq readouts (Perturb-seq) (Adamson et al., 2016; Replogle et al., 2022; Replogle et al., 2020; Tian et al., 2021; Tian et al., 2019),

In class 2 CRISPR systems, the functions of the effector complex (e.g., the cleavage of target DNA) are carried out by a single protein (which can be referred to as a CRISPR-Cas effector protein)—where the natural protein is an endonuclease (e.g., see Zetsche et al, Cell. 2015 Oct. 22; 163 (3): 759-71; Makarova et al, Nat Rev Microbiol. 2015 November; 13 (11): 722-36; Shmakov et al., Mol Cell. 2015 Nov. 5; 60 (3): 385-97; Shmakov et al., Nat Rev Microbiol. 2017 March; 15 (3): 169-182: “Diversity and evolution of class 2 CRISPR-Cas systems”; and Koonin et al., Curr Opin Microbiol. 2017 June: 37:67-78). As such, the term “class 2 CRISPR-Cas” protein or “CRISPR-Cas effector” protein is used herein to encompass the effector protein from class 2 CRISPR systems—for example, type II CRISPR-Cas proteins (e.g., Cas9), type V CRISPR-Cas proteins (e.g., Cpf1 (Cas12a), C2c1 (Cas12b), C2C3 (Cas12c), CasY (Cas12d), CasX (Cas12e), and type VI CRISPR-Cas proteins (e.g., C2c2 (Cas13a), C2C7 (Cas13c), C2c6 (Cas13b), and the like. Class 2 CRISPR-Cas effector proteins include type II, type V, and type VI CRISPR-Cas proteins, but the term is also meant to encompass any class 2 CRISPR-Cas protein suitable for binding to a corresponding guide RNA and forming a ribonucleoprotein (RNP) complex.

Examples of CRISPR-Cas effector proteins will be readily available to one of ordinary skill in the art, and any convenient CRISPR-Cas effector protein can be used. In some embodiments, a subject CRISPR-Cas effector protein will be a Cas9 protein (e.g., Staphylococcus aureus Cas9 (saCas9), Streptococcus pyogenes Cas9 (SpyCas9), Neisseria meningitidis Cas9 (nmCas9), Streptococcus thermophilus Cas9 (stCas9), etc.). In some cases, a subject CRISPR-Cas effector protein will be a Cas12a protein (e.g., Acidaminococcus sp., strain BV3L6 (AsCas12a), LbCas12a, and FnoCas12a). Sequences for these proteins are readily available to one of ordinary skill in the art. As would be understood by one of ordinary skill in the art, many variant forms of CRISPR-Cas effector proteins are known in the art, e.g., those harboring mutations that increase specificity (e.g., decrease off-targeting), and any convenient variant can be used (e.g., HiFi Cas9 and AsCas12a ultra nuclease). See, e.g., Vakulskas et al., Nat Med. 2018 August; 24 (8): 1216-1224; Kleinstiver et al., Nature. 2016 Jan. 28; 529 (7587): 490-5; Yuen et al., Nucleic Acids Res. 2022 Feb. 22; 50 (3): 1650-1660; Wei et al., FASEB J. 2023 August; 37 (8): e23060; Tan et al., Proc Natl Acad Sci USA. 2019 Oct. 15; 116 (42): 20969-20976; Kleinstiver et al., Nat Biotechnol. 2019 March; 37 (3): 276-282; DeWeirdt et al., Nat. Biotechnol. 2021 39, 94-104; and Zhang et al., Nat Commun. 2021 Jun. 23; 12 (1): 3908.

A CRISPR-Cas effector protein can be fused to a heterologous protein having any desired activity such as DNA-modifying activity (e.g., nuclease activity, methyltransferase activity, demethylase activity, DNA repair activity, DNA damage activity, deamination activity, dismutase activity, alkylation activity, depurination activity, oxidation activity, pyrimidine dimer forming activity, integrase activity, transposase activity, recombinase activity, polymerase activity, ligase activity, helicase activity, photolyase activity or glycosylase activity); transcription modulation activity (e.g., fusion to a transcription repressor or transcription activator); an activity that modifies a protein (e.g., a histone) that is associated with target DNA (e.g., methyltransferase activity, demethylase activity, acetyltransferase activity, deacetylase activity, kinase activity, phosphatase activity, ubiquitin ligase activity, deubiquitinating activity, adenylation activity, deadenylation activity, SUMOylating activity, deSUMOylating activity, ribosylation activity, deribosylation activity, myristoylation activity or demyristoylation activity).

Some activities of the heterologous protein can cause transcriptional activation (referred to as CRISPRa) while others can cause transcription repression (referred to as CRISPRi).

Examples of CRISPRi and CRISPRa fusion proteins will be known to one of ordinary skill in the art and any convenient CRISPRi or CRISPRa fusion protein can be used. A “CRISPRi fusion protein” or “CRISPRa fusion protein” includes a CRISPR-Cas effector protein (e.g., Cas9, Cas12a, dCas9, dCas12a, etc.) linked (covalently or non-covalently) to a transcription repressing or activating protein. In some cases, the CRISPR-Cas effector protein is fused to a transcription repressing protein (and is called a CRISPRi fusion protein or simply a CRISPRi protein). In some cases, the CRISPR-Cas effector protein is fused to a transcription activating protein (and is called a CRISPRa fusion protein or simply a CRISPRa protein). In some cases, the CRISPR-Cas effector protein is linked (covalently or non-covalently) directly to the transcription repressing protein (and is called a CRISPRi fusion protein or simply a CRISPRi protein). In some cases, the CRISPR-Cas effector protein is linked (covalently or non-covalently) directly to the transcription activating protein (and is called a CRISPRa fusion protein or simply a CRISPRa protein). In some cases, the CRISPR-Cas effector protein is linked (covalently or non-covalently) indirectly to the transcription repressing or activating protein, e.g., by being linked a protein that recruits a transcription repressing or activating protein (see, e.g., Griffith et al., Cell Genom. 2023 Sep. 1; 3 (9): 100387).

In some cases, the CRISPR-Cas effector protein (of the CRISPRi or CRISPRa fusion protein) harbors a mutation that reduces the endogenous nuclease activity (e.g., in some cases renders it a nickase (e.g., nCas9), and in some cases renders it catalytically inactive (“dead”), e.g., a dCas9 or dCas12a). In some cases, the CRISPR-Cas effector protein (e.g., Cas9, Cas12a) is catalytically inactive (i.e., ‘dead’), which is referred to in the art as a dCas protein (e.g., dCas9, dCas12a). Such a protein will not exhibit the nuclease cleavage activity of the Cas effector protein, but the fusion protein (the CRISPRi or CRISPRa fusion protein) will exhibit the activity of the protein to which the dCas protein is fused (i.e., the fusion partner—the transcription repressing or activating protein). Examples of mutations to produce a dCas effector protein will be known to one of ordinary skill in the art. As non-limiting examples, D10A/H840A of SpyCas9 as well as D908A and E993A of Cas12a have been employed.

Examples of proteins (or fragments thereof) that can be used to increase transcription (i.e., transcription activating proteins) include but are not limited to: transcriptional activators such as VP16, VP64, VP48, VP64, VP160, p65 subdomain (e.g., from NFkB), Rta, VPR (which is a fusion of VP64, p65, and Rta), and activation domain of EDLL and/or TAL activation domain (e.g., for activity in plants); histone lysine methyltransferases such as SET1A, SET1B, MLL1 to 5, ASH1, SYMD2, NSD1, and the like; histone lysine demethylases such as JHDM2a/b, UTX, JMJD3, and the like; histone acetyltransferases such as GCN5, PCAF, CBP, p300, TAF1, TIP60/PLIP, MOZ/MYST3, MORF/MYST4, SRC1, ACTR, P160, CLOCK, and the like; and DNA demethylases such as Ten-Eleven Translocation (TET) dioxygenase 1 (TET1CD), TET1, DME, DML1, DML2, ROS1, and the like. See, e.g., Chavez et al., Nat Methods. 2015 April; 12 (4): 326-328. In some cases, the CRISPRa system is a SAM system, which includes 3 components that form the DNA-binding complex: (1) a CRISPRa fusion protein (e.g., dCas9 fused to VP64), (2) MS2 aptamer(s) added to the guide RNA (forming a characteristic stem loop structure recognized by MS2), and (3) transcriptional activators P65 (Nuclear Factor NF-κB p65) and HSF1 (Heat Shock Factor 1) fused with an MS2-tag corresponding to the minimal aptamer-binding peptide of the MS2 coat protein. See, e.g., review articles such as Adli, Nat Commun. 2018 May 15; 9 (1): 1911; Becirovic, Cell Mol Life Sci. 2022 Feb. 12; 79 (2): 130; and Nidhi S, et al., Int J Mol Sci. 2021 Mar. 24; 22 (7): 3327.

Examples of proteins (or fragments thereof) that can be used as heterologous proteins to decrease transcription (often referred to as CRISPRi when used with a CRISPR-Cas effector protein) include but are not limited to: transcriptional repressors such as the Krüppel associated box (KRAB or SKD); ZIM3 KRAB; KOX1 repression domain; the Mad mSIN3 interaction domain (SID); the ERF repressor domain (ERD), the SRDX repression domain (e.g., for repression in plants), and the like; histone lysine methyltransferases such as Pr-SET7/8, SUV4-20H1, RIZ1, and the like; histone lysine demethylases such as JMJD2A/JHDM3A, JMJD2B, JMJD2C/GASC1, JMJD2D, JARID1A/RBP2, JARID1B/PLU-1, JARID1C/SMCX, JARID1D/SMCY, and the like; histone lysine deacetylases such as HDAC1, HDAC2, HDAC3, HDAC8, HDAC4, HDAC5, HDAC7, HDAC9, SIRT1, SIRT2, HDAC11, and the like; DNA methylases such as Hhal DNA m5c-methyltransferase (M.Hhal), DNA methyltransferase 1 (DNMT1), DNA methyltransferase 3a (DNMT3a), DNA methyltransferase 3b (DNMT3b), METI, DRM3 (plants), ZMET2, CMT1, CMT2 (plants), and the like; and periphery recruitment elements such as Lamin A, Lamin B, and the like.

In some embodiments, the CRISPR-Cas effector protein of the CRISPRi fusion protein is a dCas9. In some embodiments, the CRISPR-Cas effector protein of the CRISPRi fusion protein is a dCas12a. In some cases, the transcription repressor protein is ZIM3 KRAB (see, e.g., Alerasool et al., Nat Methods 17, 1093-1096 (2020)). In some cases, the transcription repressor protein is KOX1. A subject CRISPRa fusion protein can include one or more NLSs (e.g., two or more, three or more, etc). In some cases, the nucleotide sequence (of a nucleic acid) encoding a subject CRISPRi or CRISPRa fusion protein will be codon optimized for expression in the cell type of interest.

In some embodiments, a nucleic acid encoding the CRISPRi or CRISPRa fusion protein is integrated into the genome of the target cell of interest. In some embodiments, the nucleic acid encoding the CRISPRi or CRISPRa fusion protein is extrachromosomal.

In some cases, the nucleotide sequence encoding the CRISPRi or CRISPRa fusion protein is operably linked to a constitutive promoter. In some cases, the nucleotide sequence encoding the CRISPRi or CRISPRa fusion protein is operably linked to an inducible promoter—as such, expression modulation can be initiated by introducing the appropriate factor (e.g., doxycycline) to induce expression of the CRISPRi or CRISPRa fusion protein.

As would be understood to one of ordinary skill in the art, a CRISPRi or CRISPRa fusion protein can be introduced into a cell directly as protein. A CRISPRi or CRISPRa fusion protein can also be introduced as a nucleic encoding the protein (e.g., an RNA or a DNA such as an expression vector). In some cases, a CRISPRi or CRISPRa fusion protein is introduced as an RNA encoding the protein (and the cell translates the RNA into protein). In some cases, a CRISPRi or CRISPRa fusion protein is introduced as a DNA encoding the protein (and the cell expresses the protein via RNA transcription and translation into protein). In some cases, a sequence encoding the CRISPRi or CRISPRa fusion protein is integrated into the genome of a cell type of interest.

### Protospacer Adjacent Motif (PAM)

As would be understood by one of ordinary skill in the art, the sequence targeted by a guide RNA is generally selected taking the protospacer adjacent motif (PAM) sequence into account. The PAM sequence and position relative to the target sequence varies depending on which CRISPR-Cas effector protein is used. For example, the PAM for SpyCas9 is NGG and the PAM for SaCas9 is NNGRR(T) [both positioned downstream of the targeted sequence], while the PAM for AsCas12a is TTTV (i.e., TTTA, TTTC, or TTTG) [positioned upstream of the targeted sequence]. PAMs for enhanced versions of Cas12a can include, for example, TTYN, VTTV, TRTV, TATM, and TGTM. PAMs for many different CRISPR-Cas effector proteins are known, see, e.g., Gasiunas et al., Nat Commun. 2020 Nov. 2; 11 (1): 5512: “A catalogue of biochemically diverse CRISPR-Cas9 orthologs”. In some cases, a variant CRISPR-Cas protein (e.g., a variant Cas9 or Cas12a) protein exhibits increased PAM flexibility, i.e., has less sequence constraints with regard to the PAM, as compared to the parent Cas9 protein (e.g., the corresponding wild type Cas9 protein).

### Guide RNAs

A nucleic acid that binds to and thereby forms a ribonucleoprotein (RNP) complex with a CRISPR-Cas effector protein (e.g., a Cas9 protein; a type V or type VI CRISPR-Cas protein; a Cas12a protein; a dCas9, a dCas12a) (and thereby can also bind to a CRISPRi or CRISPRa fusion protein) and targets the complex to a specific location within a target nucleic acid is referred to herein as a “guide RNA” or “CRISPR-Cas guide nucleic acid” or “CRISPR-Cas guide RNA” or simply a “guide.” It is to be understood that in some cases, a hybrid DNA/RNA can be made such that guide RNA suitable for use in a complex with a CRISPR-Cas effector protein includes DNA bases in addition to RNA bases, but the term “guide RNA” is still used to encompass such a hybrid molecule herein.

A guide RNA can be said to include two segments, a targeting segment and a protein-binding segment. The targeting segment of a guide RNA includes a nucleotide sequence (a guide sequence) that is complementary to (and therefore hybridizes with) a specific sequence (a target site) within a target nucleic acid (e.g., a target DNA). The protein-binding segment (or “protein-binding sequence”) interacts with (binds to) a CRISPR-Cas effector protein (e.g., Cas9, dCas9, Cas12a, dCas12a). Thus, a guide RNA provides target specificity to the complex (the RNP complex) by including the targeting segment, which includes the guide sequence (the guide RNA can thereby be said to “target” a specific sequence or “target” a specific gene). A guide RNA can be referred to by the protein to which it corresponds. For example, when a CRISPR-Cas effector protein is a Cas9 protein, the corresponding guide RNA can be referred to as a “Cas9 guide RNA.” Likewise, as another example, when a CRISPR-Cas effector protein is a Cas12a protein, the corresponding guide RNA can be referred to as a “Cas12a guide RNA.”

A guide RNA can be said to “target” the gene or promoter/TSS, and one of ordinary skill in the art would understand how to design an appropriate guide RNA based on the desired outcome—i.e., they would understand what sequences within the target region to target. As noted above, a guide RNA can be said to “target” a particular gene, a particular region within a gene, or a particular sequence. For example, a guide RNA that targets a particular alternate promoter (e.g., one identified using the subject methods) has a guide sequence that hybridizes to the target DNA such that the CRISPRi or CRISPRa protein it is complexed with modulates transcription from that promoter (i.e., from the targeted promoter). In other words, a guide RNA can be said to target a particular promoter or TSS. For example, if multiple guide RNAs are said to target the same particular gene, some may target one particular TSS/promoter of that gene while others may target a different TSS/promoter. Thus, guide RNAs can be referred to as targeting a particular gene, and can also be referred to as targeting a particular promoter or TSS.

In some embodiments, a guide RNA includes two separate nucleic acid molecules: an “activator” and a “targeter” (or crRNA and tracrRNA). In some embodiments, the guide RNA is one molecule (e.g., for some class 2 CRISPR-Cas proteins, the corresponding natural guide RNA is a single molecule; and in some cases, an activator and targeter can be artificially covalently linked to one another, e.g., via intervening nucleotides), and the guide RNA is referred to as a “single guide RNA”, a “single-molecule guide RNA,” a “one-molecule guide RNA”, or simply “sgRNA.” As discussed in more detail elsewhere herein, in some cases, two or more guide RNAs can be used, e.g., to target a CRISPRi or CRISPRa fusion protein to more than one target sequence at the same time. For example, in some cases, two or more guide RNAs are used that target different sequences of a targeted promoter.

Guide RNA sequences (for the protein-binding segment) for many different CRISPR-Cas proteins (including Cas9 and Cas12a proteins) are known, see, e.g., Gasiunas et al., Nat Commun. 2020 Nov. 2; 11 (1): 5512: “A catalogue of biochemically diverse CRISPR-Cas9 orthologs.” Variant CRISPR-Cas proteins can generally use the same guide RNAs that can be used with the corresponding wild type protein. As an illustrative example, a variant Nme2Cas9 can generally use Nme2Cas9 guide RNAs while a variant SpyCas9 can generally use a SpyCas9 guide RNA.

### Guide Sequence of a Guide RNA

The targeting segment of a subject guide RNA includes a guide sequence (i.e., a targeting sequence), which is a nucleotide sequence that is complementary to a sequence (a target site) in a target nucleic acid. In other words, the targeting segment of a guide RNA can interact with a target nucleic acid (e.g., DNA) in a sequence-specific manner via hybridization (i.e., base pairing). The guide sequence of a guide RNA can be modified (e.g., by genetic engineering)/designed to hybridize to any desired target sequence (e.g., while taking the PAM into account, e.g., when targeting a dsDNA target) within a target nucleic acid (e.g., a eukaryotic target nucleic acid such as genomic DNA).

In some cases, the percent complementarity between the guide sequence and the target site of the target nucleic acid is 80% or more (e.g., 85% or more, 90% or more, 95% or more, 97% or more, 98% or more, 99% or more, or 100%). In some cases, the percent complementarity between the guide sequence and the target site of the target nucleic acid is 90% or more (e.g., 95% or more, 97% or more, 98% or more, 99% or more, or 100%). In some cases, the percent complementarity between the guide sequence and the target site of the target nucleic acid is 100%.

In some cases, the guide sequence has a length in a range of from 19-30 nucleotides (nt) (e.g., from 19-25, 19-22, 19-20, 20-30, 20-25, or 20-22 nt). In some cases, the guide sequence has a length in a range of from 19-25 nucleotides (nt) (e.g., from 19-22, 19-20, 20-25, 20-25, or 20-22 nt). In some cases, the guide sequence has a length of 19 or more nt (e.g., 20 or more, 21 or more, or 22 or more nt; 19 nt, 20 nt, 21 nt, 22 nt, 23 nt, 24 nt, 25 nt, etc.). In some cases, the guide sequence has a length of 17-18 nt. In some cases, the guide sequence has a length of 19 nt. In some cases, the guide sequence has a length of 20 nt. In some cases, the guide sequence has a length of 21 nt. In some cases, the guide sequence has a length of 22 nt. In some cases, the guide sequence has a length of 23 nt.

As would be understood to one of ordinary skill in the art, the guide RNA can be introduced into a cell as an RNA (or as a DNA/RNA hybrid) or can be introduces as a nucleic acid encoding the RNA (e.g., a DNA such as an expression vector such as a viral or plasmid DNA), in which case the cell transcribes the RNA from the introduced DNA. In some cases, the nucleotide sequence encoding the guide RNA is operably linked to a promoter (e.g., a Pol Ill promoter such as U6 or H1). As noted elsewhere herein, in some cases, one or more guide RNAs (e.g., 1, 2, 3, 4, 5, 6, 1-10, 1-8, 1-6, 1-5, 1-4, 1-3, 2-10, 2-8, 2-6, 2-5, 2-4, 3-10, 3-8, 3-6, 3-5, two or more, three or more, four or more, or five or more) (or nucleotide sequences that encode said guide RNAs) can be introduced into the same cell (e.g., to target different sequences).

### Nucleic Acid Modifications

In some embodiments, a guide RNA has one or more modifications, e.g., a base modification, a backbone modification, etc., to provide the nucleic acid with a new or enhanced feature (e.g., improved stability). A nucleoside is a base-sugar combination. The base portion of the nucleoside is normally a heterocyclic base. The two most common classes of such heterocyclic bases are the purines and the pyrimidines. Nucleotides are nucleosides that further include a phosphate group covalently linked to the sugar portion of the nucleoside. For those nucleosides that include a pentofuranosyl sugar, the phosphate group can be linked to the 2′, the 3′, or the 5′ hydroxyl moiety of the sugar. In forming oligonucleotides, the phosphate groups covalently link adjacent nucleosides to one another to form a linear polymeric compound. In turn, the respective ends of this linear polymeric compound can be further joined to form a circular compound; however, linear compounds are suitable. In addition, linear compounds may have internal nucleotide base complementarity and may therefore fold in a manner as to produce a fully or partially double-stranded compound. Within oligonucleotides, the phosphate groups are commonly referred to as forming the internucleoside backbone of the oligonucleotide. The normal linkage or backbone of RNA and DNA is a 3′ to 5′ phosphodiester linkage.

As used herein, the term “2′-modified” or “2′-substituted” means a sugar comprising a substituent at the 2′-position other than H or OH. 2′-modified nucleotides, include moieties with 2′ substituents selected from alkyl, allyl, amino, azido, fluoro, thio, O-alkyl, e.g., O-methyl, O-allyl, OCF3, O—(CH2) 2-O—CH3 (e.g., 2′-O-methoxyethyl (MOE)), O—(CH2) 2SCH3)-(CH2) 2-ONR2, and O—CH2C(O)—NR2, where each R is independently selected from H, alkyl, and substituted alkyl.

Suitable nucleic acid modifications include, but are not limited to: 2′Omethyl modified nucleotides, 2′ fluoro modified nucleotides, locked nucleic acid (LNA) modified nucleotides, peptide nucleic acid (PNA) modified nucleotides, nucleotides with phosphorothioate linkages, and a 5′ cap (e.g., a 7-methylguanylate cap (m7G)). Additional details and additional modifications are described below.

A 2′-O-Methyl modified nucleotide (also referred to as 2′-O-Methyl RNA) is a naturally occurring modification of RNA found in tRNA and other small RNAs that arises as a post-transcriptional modification. Oligonucleotides can be directly synthesized that contain 2′-O-Methyl RNA. This modification increases Tm of RNA: RNA duplexes but results in only small changes in RNA: DNA stability. It is stabile with respect to attack by single-stranded ribonucleases and is typically 5 to 10-fold less susceptible to DNases than DNA. It is commonly used in antisense oligos as a means to increase stability and binding affinity to the target message.

2′ Fluoro modified nucleotides (e.g., 2′ Fluoro bases) have a fluorine modified ribose which increases binding affinity (Tm) and also confers some relative nuclease resistance when compared to native RNA. These modifications are commonly employed in ribozymes and siRNAs to improve stability in serum or other biological fluids.

LNA bases have a modification to the ribose backbone that locks the base in the C3′-endo position, which favors RNA A-type helix duplex geometry. This modification significantly increases Tm and is also very nuclease resistant. Multiple LNA insertions can be placed in an oligo at any position except the 3′-end. Applications have been described ranging from antisense oligos to hybridization probes to SNP detection and allele specific PCR. Due to the large increase in Tm conferred by LNAs, they also can cause an increase in primer dimer formation as well as self-hairpin formation. In some cases, the number of LNAs incorporated into a single oligo is 10 bases or less.

The phosphorothioate (PS) bond (i.e., a phosphorothioate linkage) substitutes a sulfur atom for a non-bridging oxygen in the phosphate backbone of a nucleic acid (e.g., an oligo). This modification renders the internucleotide linkage resistant to nuclease degradation. Phosphorothioate bonds can be introduced between the last 3-5 nucleotides at the 5′- or 3′-end of the oligo to inhibit exonuclease degradation. Including phosphorothioate bonds within the oligo (e.g., throughout the entire oligo) can help reduce attack by endonucleases as well.

In some cases, a guide RNA has one or more nucleotides that are 2′-O-Methyl modified nucleotides. In some cases, a guide RNA has one or more 2′ Fluoro modified nucleotides. In some cases, a guide RNA has one or more LNA bases. In some cases, a guide RNA has one or more nucleotides that are linked by a phosphorothioate bond (i.e., the guide RNA has one or more phosphorothioate linkages). In some cases, guide RNA has a 5′ cap (e.g., a 7-methylguanylate cap (m7G)). In some cases, a guide RNA has a combination of modified nucleotides. For example, a guide RNA can have a 5′ cap (e.g., a 7-methylguanylate cap (m7G)) in addition to having one or more nucleotides with other modifications (e.g., a 2′-O-Methyl nucleotide and/or a 2′ fluoro modified nucleotide and/or a LNA base and/or a phosphorothioate linkage).

### Modified Backbones and Modified Internucleoside Linkages

Examples of suitable guide RNAs containing modifications include guide RNAs with modified backbones or non-natural internucleoside linkages. Guide RNA having modified backbones include those that retain a phosphorus atom in the backbone and those that do not have a phosphorus atom in the backbone.

Suitable modified nucleic acid backbones containing a phosphorus atom therein include, for example, phosphorothioates, chiral phosphorothioates, phosphorodithioates, phosphotriesters, aminoalkylphosphotriesters, methyl and other alkyl phosphonates including 3′-alkylene phosphonates, 5′-alkylene phosphonates and chiral phosphonates, phosphinates, phosphoramidates including 3′-amino phosphoramidate and aminoalkylphosphoramidates, phosphorodiamidates, thionophosphoramidates, thionoalkylphosphonates, thionoalkylphosphotriesters, selenophosphates and boranophosphates having normal 3′-5′ linkages, 2′-5′ linked analogs of these, and those having inverted polarity wherein one or more internucleotide linkages is a 3′ to 3′, 5′ to 5′ or 2′ to 2′ linkage. Suitable oligonucleotides having inverted polarity comprise a single 3′ to 3′ linkage at the 3′-most internucleotide linkage i.e. a single inverted nucleoside residue which may be a basic (the nucleobase is missing or has a hydroxyl group in place thereof). Various salts (such as, for example, potassium or sodium), mixed salts and free acid forms are also included. Nucleoside subunits can be joined by a variety of intersubunit linkages, including, but not limited to, phosphodiester, phosphotriester, an alkylphosphonate, e.g., methylphosphonate, P3′→N5′ phosphoramidate, N3′→P5′ phosphoramidate, N3′→P5′ thiophosphoramidate, phosphorodiamidate, and phosphorothioate linkages.

In certain cases, intersubunit linkage has a chiral atom. Representative chiral intersubunit linkages include, but are not limited to, alkylphosphonates, phosphorodiamidates and phosphorothioates. Further, “oligonucleotides” includes chemical and biochemical modifications, such as those known to one skilled in the art, e.g., to the sugar (e.g., 2′ substitutions), the base (see the definition of “nucleoside” below), and/or the 3′ and 5′ termini. In embodiments where the oligonucleotide moiety includes a plurality of intersubunit linkages, each linkage may be formed using the same chemistry or a mixture of linkage chemistries may be used. In embodiments where the oligonucleotide moiety includes a plurality of intersubunit linkages, one or more of the linkages may be chiral. Linkages having a chiral atom can be prepared as racemic mixtures, or as separate enantiomers.

In some cases, a guide RNA comprises one or more phosphorothioate and/or heteroatom internucleoside linkages, in particular-CH2-NH—O-CH2-, —CH2-N(CH3)-O-CH2-(known as a methylene (methylimino) or MMI backbone), —CH2-O—N(CH3)-CH2-, —CH2-N(CH3)-N(CH3)-CH2-and-O—N(CH3)-CH2-CH2-(wherein the native phosphodiester internucleotide linkage is represented as —O—P(═O) (OH)—O-CH2-). MMI type internucleoside linkages are disclosed in the above referenced U.S. Pat. No. 5,489,677, the disclosure of which is incorporated herein by reference in its entirety. Suitable amide internucleoside linkages are disclosed in U.S. Pat. No. 5,602,240, the disclosure of which is incorporated herein by reference in its entirety.

Also suitable are nucleic acids having morpholino backbone structures as described in, e.g., U.S. Pat. No. 5,034,506. For example, in some embodiments, a subject nucleic acid comprises a 6-membered morpholino ring in place of a ribose ring. In some of these embodiments, a phosphorodiamidate or other non-phosphodiester internucleoside linkage replaces a phosphodiester linkage.

Suitable modified polynucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; riboacetyl backbones; alkene containing backbones; sulfamate backbones; methyleneimino and methylenehydrazino backbones; sulfonate and sulfonamide backbones; amide backbones; and others having mixed N, O, S and CH2 component parts.

### Mimetics

A subject guide RNA can include a nucleic acid mimetic. The term “mimetic” as it is applied to polynucleotides is intended to include polynucleotides wherein only the furanose ring or both the furanose ring and the internucleotide linkage are replaced with non-furanose groups, replacement of only the furanose ring is also referred to in the art as being a sugar surrogate. The heterocyclic base moiety or a modified heterocyclic base moiety is maintained for hybridization with an appropriate target nucleic acid. One such nucleic acid, a polynucleotide mimetic that has been shown to have excellent hybridization properties, is referred to as a peptide nucleic acid (PNA). In PNA, the sugar-backbone of a polynucleotide is replaced with an amide containing backbone, in particular an aminoethylglycine backbone. The nucleotides are retained and are bound directly or indirectly to aza nitrogen atoms of the amide portion of the backbone.

One polynucleotide mimetic that has been reported to have excellent hybridization properties is a peptide nucleic acid (PNA). The backbone in PNA compounds is two or more linked aminoethylglycine units which gives PNA an amide containing backbone. The heterocyclic base moieties are bound directly or indirectly to aza nitrogen atoms of the amide portion of the backbone. Representative U.S. patents that describe the preparation of PNA compounds include, but are not limited to: U.S. Pat. Nos. 5,539,082; 5,714,331; and 5,719,262, the disclosures of which are incorporated herein by reference in their entirety.

Another class of polynucleotide mimetic that has been studied is based on linked morpholino units (morpholino nucleic acid) having heterocyclic bases attached to the morpholino ring. A number of linking groups have been reported that link the morpholino monomeric units in a morpholino nucleic acid. One class of linking groups has been selected to give a non-ionic oligomeric compound. The non-ionic morpholino-based oligomeric compounds are less likely to have undesired interactions with cellular proteins. Morpholino-based polynucleotides are non-ionic mimics of oligonucleotides which are less likely to form undesired interactions with cellular proteins (Dwaine A. Braasch and David R. Corey, Biochemistry, 2002, 41 (14), 4503-4510). Morpholino-based polynucleotides are disclosed in U.S. Pat. No. 5,034,506, the disclosure of which is incorporated herein by reference in its entirety. A variety of compounds within the morpholino class of polynucleotides have been prepared, having a variety of different linking groups joining the monomeric subunits.

A further class of polynucleotide mimetic is referred to as cyclohexenyl nucleic acids (CeNA). The furanose ring normally present in a DNA/RNA molecule is replaced with a cyclohexenyl ring. CeNA DMT protected phosphoramidite monomers have been prepared and used for oligomeric compound synthesis following classical phosphoramidite chemistry. Fully modified CeNA oligomeric compounds and oligonucleotides having specific positions modified with CeNA have been prepared and studied (see Wang et al., J. Am. Chem. Soc., 2000, 122, 8595-8602, the disclosure of which is incorporated herein by reference in its entirety). In general the incorporation of CeNA monomers into a DNA chain increases its stability of a DNA/RNA hybrid. CeNA oligoadenylates formed complexes with RNA and DNA complements with similar stability to the native complexes. The study of incorporating CeNA structures into natural nucleic acid structures was shown by NMR and circular dichroism to proceed with easy conformational adaptation.

A further modification includes Locked Nucleic Acids (LNAs) in which the 2′-hydroxyl group is linked to the 4′ carbon atom of the sugar ring thereby forming a 2′-C, 4′-C-oxymethylene linkage thereby forming a bicyclic sugar moiety. The linkage can be a methylene (—CH2-), group bridging the 2′ oxygen atom and the 4′ carbon atom wherein n is 1 or 2 (Singh et al., Chem. Commun., 1998, 4, 455-456, the disclosure of which is incorporated herein by reference in its entirety). LNA and LNA analogs display very high duplex thermal stabilities with complementary DNA and RNA (Tm=+3 to +10° C.), stability towards 3′-exonucleolytic degradation and good solubility properties. Potent and nontoxic antisense oligonucleotides containing LNAs have been described (e.g., Wahlestedt et al., Proc. Natl. Acad. Sci. U.S.A., 2000, 97, 5633-5638, the disclosure of which is incorporated herein by reference in its entirety).

The synthesis and preparation of the LNA monomers adenine, cytosine, guanine, 5-methyl-cytosine, thymine and uracil, along with their oligomerization, and nucleic acid recognition properties have been described (e.g., Koshkin et al., Tetrahedron, 1998, 54, 3607-3630, the disclosure of which is incorporated herein by reference in its entirety). LNAs and preparation thereof are also described in WO 98/39352 and WO 99/14226, as well as U.S. applications 20120165514, 20100216983, 20090041809, 20060117410, 20040014959, 20020094555, and 20020086998, the disclosures of which are incorporated herein by reference in their entirety.

A “bicyclic nucleic acid” or a “bridged nucleic acid” (BNA) refers to a modified RNA nucleotide where the ribose moiety is modified with an extra bridge connecting the 2′ oxygen and 4′ carbon, thereby forming a bicyclic ring system. BNA monomers can contain a five-membered, six-membered or a seven-membered bridge structure with a fixed 3′-endo conformation. Bridged nucleic acids include without limitation, locked nucleic acids (LNA), ethylene-bridged nucleic acids (ENA) and constrained ethyl (cEt).

A “bridge” refers to a chain of atoms or a valence bond connecting two bridgeheads, where a “bridgehead” is any skeletal atom of a ring system (e.g., the ribose ring system) which is bonded to three or more skeletal atoms (excluding hydrogen). In some embodiments, the bridge in a BNA has 7-12 ring members and 1-4 heteroatoms independently selected from nitrogen, oxygen, or sulfur. Unless otherwise specified, a BNA is optionally substituted with one or more substituents, e.g., including, but not limited to alkyl, substituted alkyl, alkoxy, substituted alkoxy, hydroxy, amino and halogen.

An “ethylene-bridged nucleic acid” (ENA) refers to an LNA modified RNA nucleotide where the ribose moiety is modified with an extra bridge containing two carbon atoms between the 2′ oxygen and the 4′ carbon (see, e.g., Morita et al., Bioorganic Medicinal Chemistry, 2003, 11 (10), 2211-2226). Ethylene-bridged nucleic acids are also encompassed by the term “bicyclic nucleic acids” or “bridged nucleic acids” (BNA).

A “constrained ethyl (cEt)” refers to an LNA modified RNA nucleotide where the ribose moiety is modified with an extra bridge connecting the 2′ oxygen and 4′ carbon, wherein the carbon atom of the bridge includes a methyl group. In some cases, the cEt is(S)-constrained ethyl. In other cases, the cEt is (R)-constrained ethyl (see, e.g., Pallan et al., Chem. Commun. (Camb)., 2012, 48 (66), 8195-8197). Constrained ethyl nucleic acids are also encompassed by the term “bicyclic nucleic acids” or “bridged nucleic acids” (BNA).

### Modified Sugar Moieties

A guide RNA can also include one or more substituted sugar moieties. Suitable polynucleotides comprise a sugar substituent group selected from: OH; F; O—, S—, or N-alkyl; O—, S—, or N-alkenyl; O—, S- or N-alkynyl; or O-alkyl-O-alkyl, wherein the alkyl, alkenyl and alkynyl may be substituted or unsubstituted C.sub.1 to C10 alkyl or C2 to C10 alkenyl and alkynyl. Particularly suitable are O((CH2)nO)mCH3, O(CH2)nOCH3, O(CH2)nNH2, O(CH2)nCH3, O(CH2)nONH2, and O(CH2)nON((CH2)nCH3)2, where n and m are from 1 to about 10. Other suitable polynucleotides comprise a sugar substituent group selected from: C1 to C10 lower alkyl, substituted lower alkyl, alkenyl, alkynyl, alkaryl, aralkyl, O-alkaryl or O-aralkyl, SH, SCH3, OCN, Cl, Br, CN, CF3, OCF3, SOCH3, SO2CH3, ONO2, NO2, N3, NH2, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. A suitable modification includes 2′-methoxyethoxy (2′-O—CH2 CH2OCH3, also known as 2′-O-(2-methoxyethyl) (or 2′-MOE or 2′-O-MOE-RNA) (Martin et al., Helv. Chim. Acta, 1995, 78, 486-504, the disclosure of which is incorporated herein by reference in its entirety) i.e., an alkoxyalkoxy group. A further suitable modification includes 2′-dimethylaminooxyethoxy, i.e., a O(CH2)2ON(CH3)2 group, also known as 2′-DMAOE, as described in examples hereinbelow, and 2′-dimethylaminoethoxyethoxy (also known in the art as 2′-O-dimethyl-amino-ethoxy-ethyl or 2′-DMAEOE), i.e., 2′-O-CH2-O-CH2-N(CH3)2.

Other suitable sugar substituent groups include methoxy (—O—CH3), aminopropoxy (—O CH2 CH2 CH2NH2), allyl (—CH2-CH═CH2), —O-allyl (—O—CH2-CH═CH2) and fluoro (F). 2′-sugar substituent groups may be in the arabino (up) position or ribo (down) position. A suitable 2′-arabino modification is 2′-F. Similar modifications may also be made at other positions on the oligomeric compound, particularly the 3′ position of the sugar on the 3′ terminal nucleoside or in 2′-5′ linked oligonucleotides and the 5′ position of 5′ terminal nucleotide. Oligomeric compounds may also have sugar mimetics such as cyclobutyl moieties in place of the pentofuranosyl sugar.

### Base Modifications and Substitutions

A guide RNA may also include nucleobase (often referred to in the art simply as “base”) modifications or substitutions. As used herein, “unmodified” or “natural” nucleobases include the purine bases adenine (A) and guanine (G), and the pyrimidine bases thymine (T), cytosine (C) and uracil (U). Modified nucleobases include other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl (—C═C—CH3) uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-amino-adenine, 8-azaguanine and 8-azaadenine, 7-deazaguanine and 7-deazaadenine and 3-deazaguanine and 3-deazaadenine. Further modified nucleobases include tricyclic pyrimidines such as phenoxazine cytidine (1H-pyrimido(5,4-b)(1,4)benzoxazin-2(3H)-one), phenothiazine cytidine (1H-pyrimido(5,4-b)(1,4)benzothiazin-2(3H)-one), G-clamps such as a substituted phenoxazine cytidine (e.g. 9-(2-aminoethoxy)-H-pyrimido(5,4-(b)(1,4)benzoxazin-2(3H)-one), carbazole cytidine (2H-pyrimido(4,5-b)indol-2-one), pyridoindole cytidine (H-pyrido(3′,2′:4,5)pyrrolo(2,3-d)pyrimidin-2-one).

Heterocyclic base moieties may also include those in which the purine or pyrimidine base is replaced with other heterocycles, for example 7-deaza-adenine, 7-deazaguanosine, 2-aminopyridine and 2-pyridone. Further nucleobases include those disclosed in U.S. Pat. No. 3,687,808, those disclosed in The Concise Encyclopedia Of Polymer Science And Engineering, pages 858-859, Kroschwitz, J. I., ed. John Wiley & Sons, 1990, those disclosed by Englisch et al., Angewandte Chemie, International Edition, 1991, 30, 613, and those disclosed by Sanghvi, Y. S., Chapter 15, Antisense Research and Applications, pages 289-302, Crooke, S. T. and Lebleu, B., ed., CRC Press, 1993; the disclosures of which are incorporated herein by reference in their entirety. Certain of these nucleobases are useful for increasing the binding affinity of an oligomeric compound. These include 5-substituted pyrimidines, 6-azapyrimidines and N-2, N-6 and O-6 substituted purines, including 2-aminopropyladenine, 5-propynyluracil and 5-propynylcytosine. 5-methylcytosine substitutions have been shown to increase nucleic acid duplex stability by 0.6-1.2° C. (Sanghvi et al., eds., Antisense Research and Applications, CRC Press, Boca Raton, 1993, pp. 276-278; the disclosure of which is incorporated herein by reference in its entirety) and are suitable base substitutions, e.g., when combined with 2′-O-methoxyethyl sugar modifications.

### Conjugates

Another possible modification of a guide RNA involves chemically linking to the polynucleotide one or more moieties or conjugates which enhance the activity, cellular distribution or cellular uptake of the guide RNA. These moieties or conjugates can include conjugate groups covalently bound to functional groups such as primary or secondary hydroxyl groups. Conjugate groups include, but are not limited to, intercalators, reporter molecules, polyamines, polyamides, polyethylene glycols, polyethers, groups that enhance the pharmacodynamic properties of oligomers, and groups that enhance the pharmacokinetic properties of oligomers. Suitable conjugate groups include, but are not limited to, cholesterols, lipids, phospholipids, biotin, phenazine, folate, phenanthridine, anthraquinone, acridine, fluoresceins, rhodamines, coumarins, and dyes. Groups that enhance the pharmacodynamic properties include groups that improve uptake, enhance resistance to degradation, and/or strengthen sequence-specific hybridization with the target nucleic acid. Groups that enhance the pharmacokinetic properties include groups that improve uptake, distribution, metabolism or excretion of a subject nucleic acid.

Conjugate moieties include but are not limited to lipid moieties such as a cholesterol moiety (Letsinger et al., Proc. Natl. Acad. Sci. USA, 1989, 86, 6553-6556), cholic acid (Manoharan et al., Bioorg. Med. Chem. Let., 1994, 4, 1053-1060), a thioether, e.g., hexyl-S-tritylthiol (Manoharan et al., Ann. N.Y. Acad. Sci., 1992, 660, 306-309; Manoharan et al., Bioorg. Med. Chem. Let., 1993, 3, 2765-2770), a thiocholesterol (Oberhauser et al., Nucl. Acids Res., 1992, 20, 533-538), an aliphatic chain, e.g., dodecandiol or undecyl residues (Saison-Behmoaras et al., EMBO J., 1991, 10, 1111-1118; Kabanov et al., FEBS Lett., 1990, 259, 327-330; Svinarchuk et al., Biochimie, 1993, 75, 49-54), a phospholipid, e.g., di-hexadecyl-rac-glycerol or triethylammonium 1,2-di-O-hexadecyl-rac-glycero-3-H-phosphonate (Manoharan et al., Tetrahedron Lett., 1995, 36, 3651-3654; Shea et al., Nucl. Acids Res., 1990, 18, 3777-3783), a polyamine or a polyethylene glycol chain (Manoharan et al., Nucleosides & Nucleotides, 1995, 14, 969-973), or adamantane acetic acid (Manoharan et al., Tetrahedron Lett., 1995, 36, 3651-3654), a palmityl moiety (Mishra et al., Biochim. Biophys. Acta, 1995, 1264, 229-237), or an octadecylamine or hexylamino-carbonyl-oxycholesterol moiety (Crooke et al., J. Pharmacol. Exp. Ther., 1996, 277, 923-937)

### Methods of Modulating Gene Expression

Provided are methods of modifying expression of a target gene. Such methods include introducing into or expressing in a target cell (e.g., a eukaryotic cell) a guide RNA designed using a subject method. In some cases, the guide RNA(s) are introduced as RNA (i.e., do not need to be expressed). In some cases, the guide RNA(s) are introduced as DNA (i.e., they are expressed by the cell after introduction into the cell).

Guide RNAs (or nucleic acids encoding them) can be introduced into any desired target cell—see, e.g., any of the target cells (cell types of interest) discussed above. Generally, the target cell with be the same cell type as the cell type of interest that was used for guide RNA design (i.e., the same cell type from which the TSS expression data were derived).

In some cases, a library of guide RNAs are introduced into a population of cells. In such methods, that target cell(s) expresses a CRISPRi or CRISPRa effector protein, and the method therefore causes modulation of gene expression in the target cell(s). In some cases, all guide RNAs targeting a given gene (e.g., targeting two or more different TSS expression peaks or multiple guide RNAs targeting the same expression peak) are introduced into (or expressed in) the same cell. In some such cases, the guide RNAs (e.g., 2 or more, 3 or more, 4 or more, 5 or more) targeting alternative and/or canonical promoters of the same target gene are expressed from the same nucleic acid. In some cases, different guide RNAs that target the same gene (e.g., targeting two or more different TSS expression peaks or multiple guide RNAs targeting the same expression peak) are introduced into (or expressed in) different cells.

As noted herein, in the subject methods, a subject nucleic acid (e.g., one that encodes one or more guide RNAs) can be introduced into a target cell (i.e., delivered to a target cell) (e.g., administered as a guide RNA library to a population of cells). In some embodiments a CRISPR-Cas effector protein (or CRISPRi or CRISPRa fusion protein) is also delivered to a target cell (in some cases as DNA, in some cases as RNA, and in some cases as protein).

As would be readily understood by one of ordinary skill in the art, subject nucleic acids (e.g., vectors) and proteins can be delivered to cells using any convenient method. Vectors may be provided directly to a target host cell (target cell). In other words, the cells are contacted with vectors comprising the subject nucleic acids (e.g., expression vectors) such that the vectors are taken up by the cells. Methods for contacting cells with nucleic acid vectors (e.g., plasmids) include electroporation, calcium chloride transfection, microinjection, and lipofection, which are well known in the art. For viral vector delivery, cells can be contacted with viral particles comprising the subject viral expression vectors (e.g., adeno-associated virus (AAV)).

In some embodiments, a subject vector is a viral construct, e.g., a recombinant adeno-associated virus construct (see, e.g., U.S. Pat. No. 7,078,387), a recombinant adenoviral construct, a recombinant lentiviral construct, a recombinant retroviral construct, etc.

Suitable expression vectors include, but are not limited to, viral vectors (e.g. viral vectors based on vaccinia virus; poliovirus; adenovirus (see, e.g., Li et al., Invest Opthalmol Vis Sci 35:2543 2549, 1994; Borras et al., Gene Ther 6:515 524, 1999; Li and Davidson, PNAS 92:7700 7704, 1995; Sakamoto et al., H Gene Ther 5:1088 1097, 1999; WO 94/12649, WO 93/03769; WO 93/19191; WO 94/28938; WO 95/11984 and WO 95/00655); adeno-associated virus (see, e.g., Ali et al., Hum Gene Ther 9:81 86, 1998, Flannery et al., PNAS 94:6916 6921, 1997; Bennett et al., Invest Opthalmol Vis Sci 38:2857 2863, 1997; Jomary et al., Gene Ther 4:683 690, 1997, Rolling et al., Hum Gene Ther 10:641 648, 1999; Ali et al., Hum Mol Genet 5:591 594, 1996; Srivastava in WO 93/09239, Samulski et al., J. Vir. (1989) 63:3822-3828; Mendelson et al., Virol. (1988) 166:154-165; and Flotte et al., PNAS (1993) 90:10613-10617); SV40; herpes simplex virus; human immunodeficiency virus (see, e.g., Miyoshi et al., PNAS 94:10319 23, 1997; Takahashi et al., J Virol 73:7812 7816, 1999); a retroviral vector (e.g., Murine Leukemia Virus, spleen necrosis virus, and vectors derived from retroviruses such as Rous Sarcoma Virus, Harvey Sarcoma Virus, avian leukosis virus, a lentivirus, human immunodeficiency virus, myeloproliferative sarcoma virus, and mammary tumor virus); and the like.

Retroviruses, for example, lentiviruses, are suitable for use in methods of the present disclosure. Commonly used retroviral vectors are “defective”, i.e. unable to produce viral proteins required for productive infection. Rather, replication of the vector requires growth in a packaging cell line. To generate viral particles comprising nucleic acids of interest, the retroviral nucleic acids comprising the nucleic acid are packaged into viral capsids by a packaging cell line. Different packaging cell lines provide a different envelope protein (ecotropic, amphotropic or xenotropic) to be incorporated into the capsid, this envelope protein determining the specificity of the viral particle for the cells (ecotropic for murine and rat; amphotropic for most mammalian cell types including human, dog and mouse; and xenotropic for most mammalian cell types except murine cells). The appropriate packaging cell line may be used to ensure that the cells are targeted by the packaged viral particles. Methods of introducing subject vector expression vectors into packaging cell lines and of collecting the viral particles that are generated by the packaging lines are well known in the art. Nucleic acids can also introduced by direct micro-injection (e.g., injection of RNA).

Methods of introducing nucleic acids and/or proteins into a target cell are known in the art, and any convenient method can be used. Suitable methods include, e.g., viral infection (e.g., AAV, adenovirus, lentiviral), transfection, conjugation, protoplast fusion, lipofection, electroporation, calcium phosphate precipitation, polyethyleneimine (PEI)-mediated transfection, DEAE-dextran mediated transfection, liposome-mediated transfection, particle gun technology, calcium phosphate precipitation, direct micro injection, nanoparticle-mediated nucleic acid delivery (see, e.g., Panyam et., al Adv Drug Deliv Rev. 2012 Sep. 13. pii: S0169-409X (12) 00283-9), and the like.

A subject protein can be introduced into a cell (provided to the cell) by any convenient method; such methods are known to those of ordinary skill in the art. As an illustrative example, a subject protein can be injected directly into a cell. As another example, a subject protein can be introduced into a cell via nucleofection; via a protein transduction domain (PTD) conjugated to the protein, etc.

In some cases, a subject nucleic acid and/or protein is delivered to a target cell in a particle, or associated with a particle. In some cases, a subject protein is delivered with a cationic lipid and a hydrophilic polymer, for instance wherein the cationic lipid comprises 1,2-dioleoyl-3-trimethylammonium-propane (DOTAP) or 1,2-ditetradecanoyl-sn-glycero-3-phosphocholine (DMPC) and/or wherein the hydrophilic polymer comprises ethylene glycol or polyethylene glycol (PEG); and/or wherein the particle further comprises cholesterol (e.g., particle from formulation 1=DOTAP 100, DMPC 0, PEG 0, Cholesterol 0; formulation number 2=DOTAP 90, DMPC 0, PEG 10, Cholesterol 0; formulation number 3=DOTAP 90, DMPC 0, PEG 5, Cholesterol 5).

A subject nucleic acid and/or protein may be delivered using particles or lipid envelopes. For example, a biodegradable core-shell structured nanoparticle with a poly (β-amino ester) (PBAE) core enveloped by a phospholipid bilayer shell can be used. In some cases, particles/nanoparticles based on self assembling bioadhesive polymers are used; such particles/nanoparticles may be applied to oral delivery of peptides, intravenous delivery of peptides and nasal delivery of peptides, e.g., to the brain. Other embodiments, such as oral absorption and ocular delivery of hydrophobic drugs are also contemplated. A molecular envelope technology, which involves an engineered polymer envelope which is protected and delivered to the site of the disease, can be used.

Lipidoid compounds (e.g., as described in U.S. patent application No. 20110293703) are also useful in the administration of polynucleotides, and can be used to deliver a subject protein (or RNA or DNA encoding it). In one aspect, the aminoalcohol lipidoid compounds are combined with an agent to be delivered to a cell or a subject to form microparticles, nanoparticles, liposomes, or micelles. The aminoalcohol lipidoid compounds may be combined with other aminoalcohol lipidoid compounds, polymers (synthetic or natural), surfactants, cholesterol, carbohydrates, proteins, lipids, etc. to form the particles. These particles may then optionally be combined with a pharmaceutical excipient to form a pharmaceutical composition.

A poly (beta-amino alcohol) (PBAA) can be used to deliver a subject protein or nucleic acid to a target cell. US Patent Publication No. 20130302401 relates to a class of poly(beta-amino alcohols) (PBAAs) that has been prepared using combinatorial polymerization.

Sugar-based particles may be used, for example GalNAc, as described with reference to WO2014118272 (incorporated herein by reference) and Nair, J K et al., 2014, Journal of the American Chemical Society 136 (49), 16958-16961) can be used to deliver a subject protein or nucleic acid to a target cell.

In some cases, lipid nanoparticles (LNPs) are used to deliver a subject nucleic acid and/or protein to a target cell. Preparation of LNPs will be known to one of ordinary skill in the art, e.g., as described in, e.g., Rosin et al. (2011) Molecular Therapy 19:1286-2200). As non-limiting examples, cationic lipids 1,2-dilineoyl-3-dimethylammonium-propane (DLinDAP), 1,2-dilinoleyloxy-3-N,N-dimethylaminopropane (DLinDMA), 1,2-dilinoleyloxyketo-N,N-dimethyl-3-aminopropane (DLink-DMA), 1,2-dilinoleyl-4-(2-dimethylaminoethyl)-[1,3]-dioxolane (DLinKC2-DMA), (3-o-[2″-(methoxypolyethyleneglycol 2000) succinoyl]-1,2-dimyristoyl-sn-glycol (PEG-S-DMG), and R-3-[(.omega.-methoxy-poly(ethylene glycol) 2000) carbamoyl]-1,2-dimyristyloxlpropyl-3-amine (PEG-C-DOMG) may be used. A nucleic acid may be encapsulated in LNPs containing DLinDAP, DLinDMA, DLink-DMA, and DLinKC2-DMA (cationic lipid: DSPC: CHOL: PEGS-DMG or PEG-C-DOMG at 40:10:40:10 molar ratios). In some cases, SP-DiOC18 (e.g., 0.2% SP-DiOC18) is incorporated.

### Computers

Also provided are one or more computational systems (e.g., a computer) that may be used in the methods and compositions of the present disclosure. In some embodiments, a computational system of the present disclosure may be used to perform the assessing step(s) and/or the processing step(s) of a subject method. In some embodiments, a computational system of the present disclosure may be used to merge/compile TSS expression data with genome annotation data, exon annotation data, and/or guide RNA sequence data (e.g., from a CRISPRi v3 guide RNA library) to generate quantitative data relating to active promoters and their relative genomic location (e.g., to generate a file including the merged data). In some embodiments, a computational system of the present disclosure may be used to process the quantitative data relating to active promoters and their relative genomic location to identify genes for which the most highly utilized promoter is responsible for at least a threshold percentage of transcription. Such processing can also include applying a criteria that assure that a selected promoter (selected for targeting) is not the primary promoter of the target gene as identified by the FANTOM5 project or as targeted by CRISPRi v3 guide RNA library. In some embodiments, a computational system of the present disclosure may be configured to identify, for each of a plurality of genes, the most highly utilized promoter that is responsible for at least a threshold percentage of transcription. Each computational system of the present disclosure may perform one or more functions as described above.

A computational unit may include any suitable components to perform the functions as described above. Thus, the computational unit may include one or more of the following: a processor; a non-transient, computer-readable memory, such as a computer-readable medium; an input device, such as a keyboard, mouse, touchscreen, etc.; an output device, such as a monitor, screen, speaker, etc.; a network interface, such as a wired or wireless network interface; and the like.

Raw data, such as the number of reads for each gene and/or TSS peak, and the like, can be analyzed and stored on a computer-based system. As used herein, “a computer-based system” refers to the hardware means, software means, and data storage means used to analyze the information of the present invention. The minimum hardware of the computer-based systems of the present invention comprises a central processing unit (CPU), input means, output means, and data storage means. A skilled artisan can readily appreciate that any one of the currently available computer-based system are suitable for use in the present disclosure. The data storage means may comprise any manufacture comprising a recording of the present information as described above, or a memory access means that can access such a manufacture.

Performance of the described functions may be implemented in hardware or software, or a combination of both. In one embodiment of the invention, a machine-readable storage medium is provided, the medium comprising a data storage material encoded with machine readable data which, when using a machine programmed with instructions for using said data, is capable of displaying a any of the datasets and data comparisons of the present disclosure. In some embodiments, the function is implemented in computer programs executing on programmable computers, comprising a processor, a data storage system (including volatile and non-volatile memory and/or storage elements), at least one input device, and at least one output device. Program code is applied to input data to perform the functions described above and generate output information. The output information is applied to one or more output devices, in known fashion. The computer may be, for example, a personal computer, microcomputer, or workstation of conventional design.

The computer also comprises a program of instructions. The program of instructions can comprise a machine learning algorithm used for any of the above assessing or processing steps. Any machine learning algorithms deemed useful may be used. Useful machine learning algorithms include, without limitation, two-cluster Gaussian mixture models, logistic regression, random forest, gradient boosted tree, support vector machine, linear/quadratic discriminant analysis, k nearest neighbors, naïve bayes, neural network, etc. More details are provided elsewhere herein.

Each program is preferably implemented in a high level procedural or object oriented programming language to communicate with a computer system. However, the programs can be implemented in assembly or machine language, if desired. In any case, the language can be a compiled or interpreted language. Each such computer program is preferably stored on a storage media or device (e.g., ROM or magnetic diskette) readable by a general or special purpose programmable computer, for configuring and operating the computer when the storage media or device is read by the computer to perform the procedures described herein. The system can also be considered to be implemented as a computer-readable storage medium, configured with a computer program, where the storage medium so configured causes a computer to operate in a specific and predefined manner to perform the functions described herein.

A variety of structural formats for the input and output means can be used to input and output the information in the computer-based systems of the present invention. One format for an output means test datasets possessing varying degrees of similarity to a trusted profile. Such presentation provides a skilled artisan with a ranking of similarities and identifies the degree of similarity contained in the test pattern.

The data and analysis thereof can be provided in a variety of media to facilitate their use. “Media” refers to a manufacture that contains the signature pattern information of the present disclosure. The data of the present disclosure can be recorded on computer readable media, e.g. any medium that can be read and accessed directly by a computer. Such media include, but are not limited to: magnetic storage media, such as floppy discs, hard disc storage medium, and magnetic tape; optical storage media such as CD-ROM; electrical storage media such as RAM and ROM; and hybrids of these categories such as magnetic/optical storage media. One of skill in the art can readily appreciate how any of the presently known computer readable mediums can be used to create a manufacture comprising a recording of the present data and information. “Recorded” refers to a process for storing information on computer readable medium, using any such methods as known in the art. Any convenient data storage structure can be chosen, based on the means used to access the stored information. A variety of data processor programs and formats can be used for storage, e.g. word processing text file, database format, etc.

Further provided herein is a method of storing and/or transmitting, via computer, sequence, and other, data collected by the methods disclosed herein. Any computer or computer accessory including, but not limited to software and storage devices, can be utilized to practice the present disclosure. Sequence or other data can be input into a computer by a user either directly or indirectly. Additionally, any devices which can be used to sequence nucleic acid (e.g., DNA) or analyze nucleic acid (e.g., DNA) can be linked to a computer, such that the data is transferred to a computer and/or computer-compatible storage device. Data can be stored on a computer or suitable storage device (e.g., CD). Data can also be sent from a computer to another computer or data collection point via methods well known in the art (e.g., the internet, ground mail, air mail). Thus, data collected by the methods described herein can be collected at any point or geographical location and sent to any other geographical location.

### Exemplary Non-Limiting Aspects of the Disclosure

Aspects, including embodiments, of the present subject matter described above may be beneficial alone or in combination, with one or more other aspects or embodiments. Without limiting the foregoing description, certain non-limiting aspects of the disclosure are provided below. As will be apparent to those of ordinary skill in the art upon reading this disclosure, each of the individually numbered aspects may be used or combined with any of the preceding or following individually numbered aspects. This is intended to provide support for all such combinations of aspects and is not limited to combinations of aspects explicitly provided below. It will be apparent to one of ordinary skill in the art that various changes and modifications can be made without departing from the spirit or scope of the invention.
1. A method of identifying promoters for targeted expression modulation, the method comprising:
   (a) assessing, for a cell type of interest, using a computer:
      (i) transcript start site (TSS) expression data, and
      (ii) genome annotation data, thereby generating quantitative data relating to active promoters and their relative genomic location;
   (b) computer processing said quantitative data to identify, for each of a plurality of genes, the most highly utilized promoter that is responsible for at least a threshold percentage of transcription,
   thereby identifying promoters for targeted expression modulation for the cell type of interest.
2. The method of 1, wherein at least one of said promoters for targeted modulation is an alternative promotor and this therefore not a primary promoter as identified by the FANTOM5 project or as targeted by CRISPRi v3 guide RNA library.
3. The method of 1, wherein said promoters for targeted modulation are alternative promotors and are therefore not primary promoters identified by the FANTOM5 project or targeted by CRISPRi v3 guide RNA library.
4. A method of identifying one or more target genes for alternative promoter targeting, the method comprising:
   (a) assessing, for a cell type of interest, using a computer:
      (i) transcript start site (TSS) expression data, and
      (ii) genome annotation data,
      thereby generating quantitative data relating to active promoters and their relative genomic location;
   (b) computer processing said quantitative data to identify genes for which the most highly utilized promoter is responsible for at least a threshold percentage of transcription and is not the primary promoter of the target gene as identified by the FANTOM5 project or as targeted by CRISPRi v3 guide RNA library,
   thereby identifying target genes for alternative promoter targeting.
5. The method of any one of 1-4, wherein the genome annotation data comprises genomic location data for gene annotations and for known CRISPR-Cas guide RNA targets.
6. The method of 5, wherein the genome annotation data further comprises genomic location data for exon annotations.
7. The method of 5 or 6, wherein the most highly utilized promoter is at least a threshold distance away from the closest known CRISPR-Cas guide RNA target.
8. The method of 7, wherein said closest known CRISPR-Cas guide RNA is from CRISPRi v3 guide RNA library.
9. The method of 7 or 8, wherein the threshold distance away from the closest known CRISPR-Cas guide RNA target is 3 kilobases (kb).
10. The method of 7 or 8, wherein the threshold distance away from the closest known CRISPR-Cas guide RNA target is 5 kilobases (kb).
11. The method of any one of 1-10, wherein the threshold percentage of transcription is 40%.
12. The method of any one of 1-10, wherein the threshold percentage of transcription is 50%.
13. The method of any one of 1-10, wherein the threshold percentage of transcription is 60%.
14. The method of any one of 1-13, wherein the TSS expression data comprises cap analysis of gene expression (CAGE) data.
15. The method of any one of 1-14, wherein the TSS expression data includes total read counts per expression peak.
16. The method of any one of 1-15, wherein the quantitative data relating to active promoters and their relative genomic location includes expression peak data and the closest gene annotation, the closest exon annotation, and/or the closest CRISPR-Cas guide RNA target annotation for each expression peak.
17. The method of any one of 1-16, wherein the computer processing comprises removing expression peaks for which the closest gene annotation is greater than 500 base pairs away.
18. The method of any one of 1-17, wherein the computer processing comprises removing expression peaks that are within 10% of the 3′ end of a gene.
19. The method of any one of 1-18, wherein the computer processing comprises ranking expression peaks for each gene based on the ratio of read counts for each expression peak to the total read counts for all expression peaks for a given gene. (i.e., the percentage of reads for each expression peak).
20. The method of any one of 1-19, further comprising a step of generating the TSS expression data.
21. The method of any one of 1-20, further comprising a step of designing CRISPR-Cas guide RNAs to target CRISPRi or CRISPRa effector polypeptide to alternative promoters of the identified target genes.
22. The method of 21, further comprising a step of producing the designed CRISPR-Cas guide RNAs.
23. The method of any one of 21-22, wherein the CRISPR-Cas guide RNAs are Cas9 guide RNAs.
24. The method of any one of 21-22, wherein the CRISPR-Cas guide RNAs are Cas12a guide RNAs.
25. The method of 24, wherein the Cas12a guide RNAs are encoded by DNA vectors that each encode more than two of the Cas12a guide RNAs.
26. The method of any one of 21-24, wherein the CRISPR-Cas guide RNAs are encoded by DNA vectors that each encode two or more of the CRISPR-Cas guide RNAs.
27. The method of any one of 1-26, wherein the cell type of interest is a mouse cell, a non-human primate cell, or a human cell.
28. The method of any one of 1-26, wherein the cell type of interest is an immortalized cell line.
29. The method of 28, wherein the immortalized cell line is an immortalized mouse cell line, an immortalized non-human primate cell line, or an immortalized human cell line.
30. A promoter-targeted CRISPR-Cas guide RNA library, comprising a plurality of CRISPR-Cas guide RNAs or nucleic acids encoding the plurality of CRISPR-Cas guide RNAs, wherein the CRISPR-Cas guide RNAs are targeted to said most highly utilized promoters of any one of 1-29.
31. The promoter-targeted CRISPR-Cas guide RNA library of 30, wherein said library comprises at least one CRISPR-Cas guide RNA targeted to an alternative promoter, which is not a primary promoter identified by the FANTOM5 project or as targeted by CRISPRi v3 guide RNA library.
32. The promoter-targeted CRISPR-Cas guide RNA library of 30, wherein said library does not include CRISPR-Cas guide RNAs that are targeted to promoters identified by the FANTOM5 project or as targeted by CRISPRi v3 guide RNA library.
33. The promoter-targeted CRISPR-Cas guide RNA library of 30, wherein said library comprises, for each target gene:
   (i) a CRISPR-Cas guide RNA targeted to an alternative promoter, which is not a primary promoter identified by the FANTOM5 project or as targeted by CRISPRi v3 guide RNA library; and
   (ii) a CRISPR-Cas guide RNA targeted to a primary promoter identified by the FANTOM5 project or as targeted by CRISPRi v3 guide RNA library.
34. The promoter-targeted CRISPR-Cas guide RNA library of any one of 30-33, wherein the library comprises, for each of 10,000 to 30,000 genes of a genome of interest:
   (i) a CRISPR-Cas guide RNA targeted to an alternative promoter, which is not a primary promoter identified by the FANTOM5 project or as targeted by CRISPRi v3 guide RNA library; and
   (ii) a CRISPR-Cas guide RNA targeted to a primary promoter identified by the FANTOM5 project or as targeted by CRISPRi v3 guide RNA library.
35. A method of modulating expression of a target gene, the method comprising introducing into a cell or expressing in the cell:
   (a) one or more CRISPR-Cas guide RNAs targeted to an alternative promoter of a target gene; and
   (b) one or more CRISPR-Cas guide RNAs targeted to a primary promoter of the target gene, wherein the primary promoter was identified by the FANTOM5 project or as targeted by CRISPRi v3 guide RNA library,
   wherein the cell expresses a CRISPRi or CRISPRa effector polypeptide,
   thereby modulating expression of the target gene.
36. The method of 35, wherein the target gene is identified by the method of any one of 4-29.
37. The method of 35 or 36, wherein expression of two or more target genes is modulated by introducing CRISPR-Cas guide RNAs targeted to an alternative promoter and a primary promoter of each target gene.
38. The method of 37, wherein the alternative promoters of the two or more target genes is identified by the method of any one of 4-29.
39. The method of any one of 35-38, wherein the CRISPRi or CRISPRa effector polypeptide is a Cas9 CRISPRi or CRISPRa effector polypeptide.
40. The method of any one of 35-38, wherein the CRISPRi or CRISPRa effector polypeptide is a Cas12a CRISPRi or CRISPRa effector polypeptide.
41. The method of 40, wherein four or more CRISPR-Cas guide RNAs targeting alternative and/or canonical promoters of the same target gene are expressed from the same nucleic acid.

### EXPERIMENTAL EXAMPLES

The following examples are provided for purposes of illustration only, and are not intended to be limiting unless otherwise specified. Thus, the invention should in no way be construed as being limited to the following examples, but rather should be construed to encompass any and all variations which become evident as a result of the teaching provided herein.

Without further description, it is believed that one of ordinary skill in the art can, using the preceding description and the following illustrative examples, make and utilize the present invention and practice the claimed methods. The following working examples therefore are not to be construed as limiting in any way the remainder of the disclosure.

General methods in molecular and cellular biochemistry can be found in such standard textbooks as Molecular Cloning: A Laboratory Manual, 3rd Ed. (Sambrook et al., HaRBor Laboratory Press 2001); Short Protocols in Molecular Biology, 4th Ed. (Ausubel et al. eds., John Wiley & Sons 1999); Protein Methods (Bollag et al., John Wiley & Sons 1996); Nonviral Vectors for Gene Therapy (Wagner et al. eds., Academic Press 1999); Viral Vectors (Kaplift & Loewy eds., Academic Press 1995); Immunology Methods Manual (I. Lefkovits ed., Academic Press 1997); and Cell and Tissue Culture: Laboratory Procedures in Biotechnology (Doyle & Griffiths, John Wiley & Sons 1998), the disclosures of which are incorporated herein by reference. Reagents, cloning vectors, cells, and kits for methods referred to in, or related to, this disclosure are available from commercial vendors such as BioRad, Agilent Technologies, Thermo Fisher Scientific, Sigma-Aldrich, New England Biolabs (NEB), Takara Bio USA, Inc., and the like, as well as repositories such as e.g., Addgene, Inc., American Type Culture Collection (ATCC), and the like.

### Example 1: Identification of Alternative Promoter Usage

The existing best-in-class CRISPRi library (Replogle et al., eLife. 2022 Dec. 28:11:e81856), here referred to as the CRISPRi v3 library, achieves significant transcriptional knockdown across the majority of genes, but suffers from poor knockdown in a subset of targeted genes. The discovery power of this library could be greatly improved by understanding the reason underlying poor knockdown in this subset of genes and identifying methods for rescue. We developed a hypothesis that the observed poor transcriptional knockdown is in part due to ineffective promoter targeting, with the v3 library targeting canonical P1/P2 promoters, which do not accurately capture active promoter usage across all cell lines. To test this hypothesis, we developed methods to identify cell line-specific alternative promoter usage and designed novel CRISPRi guides against these promoters. We then ran Perturb-seq experiments to measure transcriptional knockdown effected by guides targeting either the canonical P1/P2 promoters or the newly identified alternative promoters.

A custom computational script was developed to identify alternative transcriptional start sites in the target cell line of K562, an immortalized human leukemia cell line. Briefly, K562 CAGE data was imported from the FANTOM Consortium dataset, active promoters identified, and genes for which the most highly used promoter is over 5 kilobases away from the closest CRISPRi v3 guide (Replogle et al., eLife. 2022 Dec. 28:11:e81856) and responsible for at least 60% of transcriptional reads selected as alternative promoters. A more detailed workflow can be found in FIG. 1.

### Example 2: Experimental Design

Proof of concept screens were run in a K562 cell line lentivirally transduced with a catalytically inactive Cas9 enzyme (dCas9) coupled to either a KOX1 or ZIM3 KRAB transcriptional repression domain (CRISPRi). Previous CRISPRi studies in K562 utilized the KOX1 repression domain (Replogle et al., eLife. 2022 Dec. 28:11:e81856), but subsequent work has suggested that ZIM3 is a more potent repressor. Experiments were run using both the KOX1 and ZIM3 domains in order to assess ZIM3 performance relative to KOX1.

After computational identification of alternative promoter usage in K562 based on CAGE data, new CRISPRi guides were designed against these promoters. The CRISPRi v3 guide designs were based on a previously published machine-learning algorithm (Horlbeck et al., Elife. 2016 Sep. 23:5:e19760). However, the published script has not been maintained in several years, and initial attempts to use this script to recapitulate CRISPRi v3 guide predictions and rankings were not successful. Consequently, CRISPick, a CRISPR guide prediction algorithm published by the Broad Institute, was used to design the CRIPRi v3 guides.

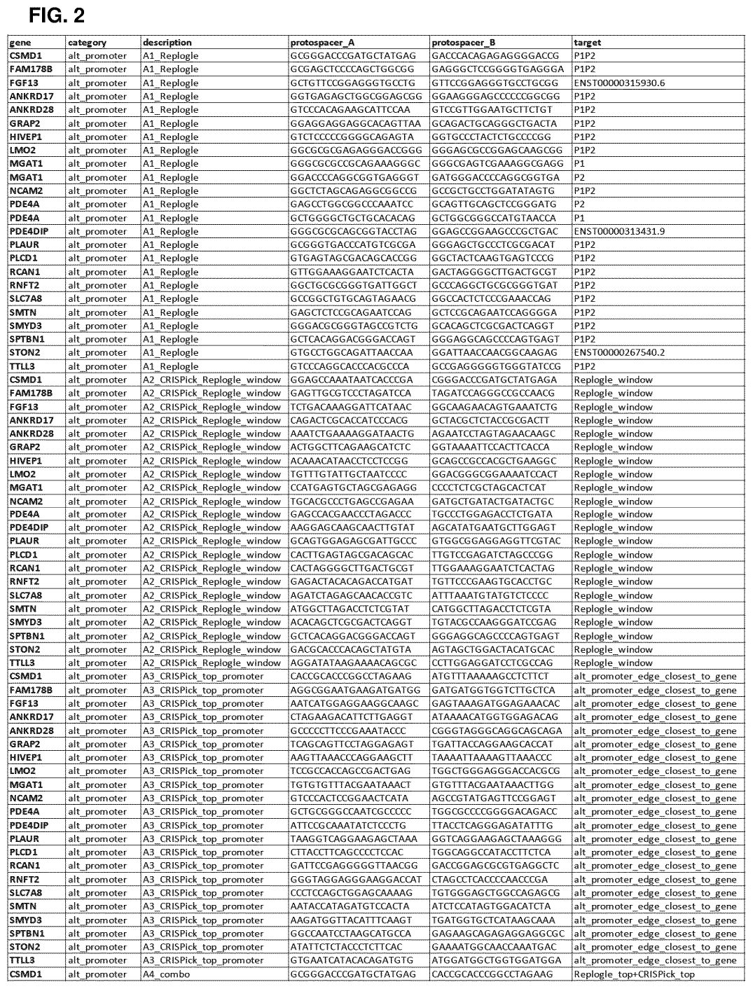

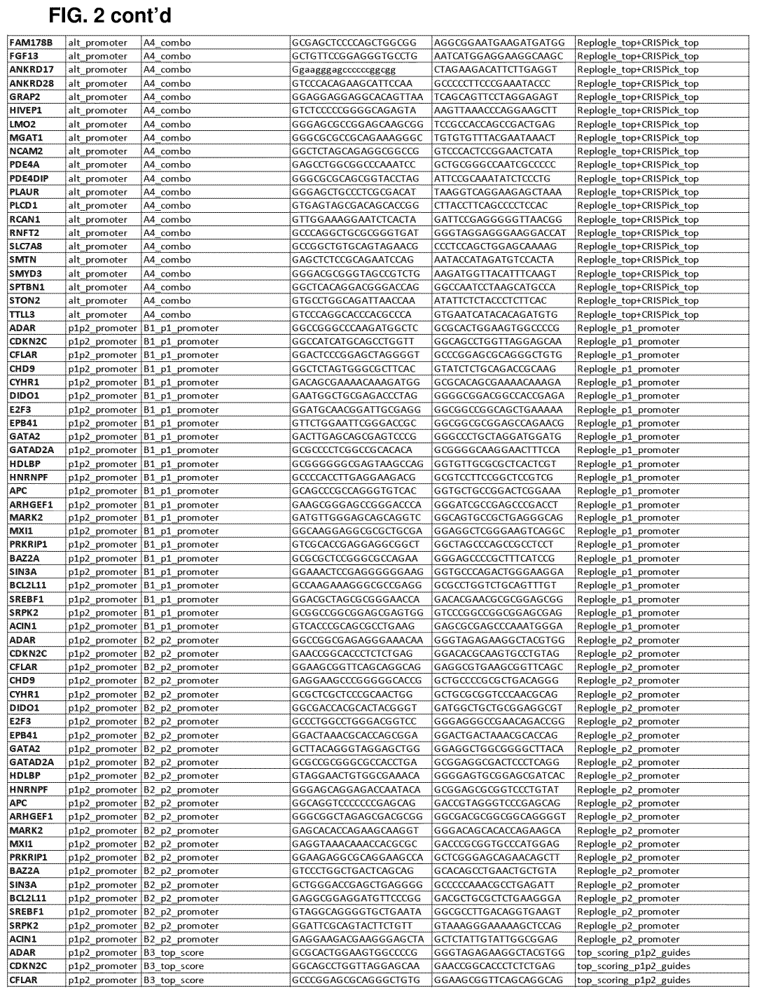

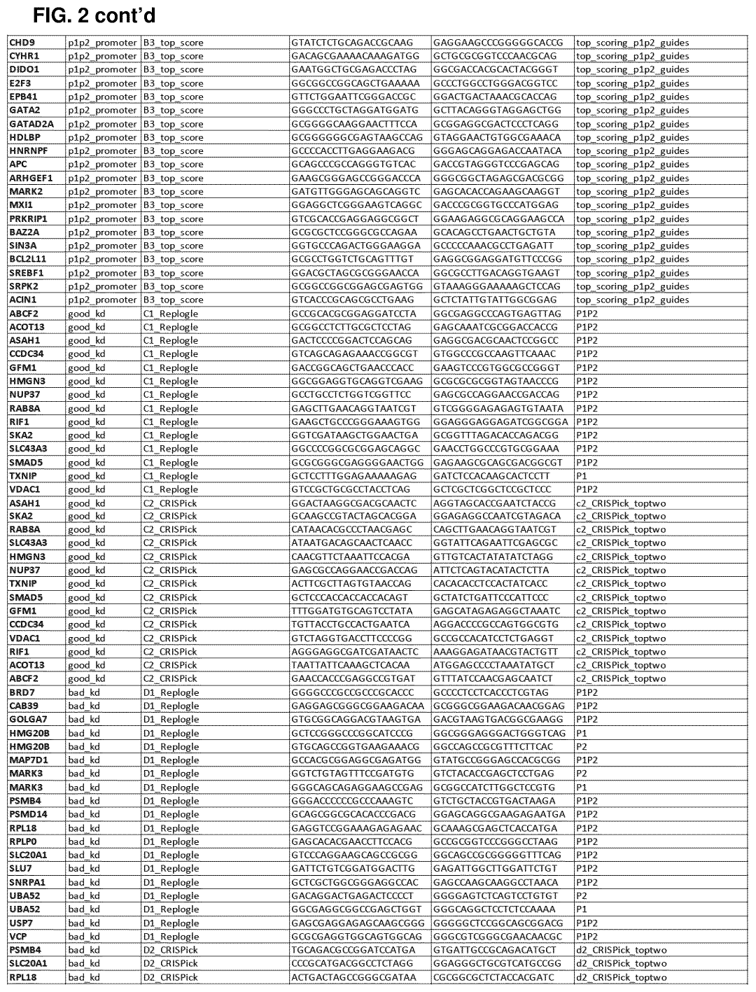

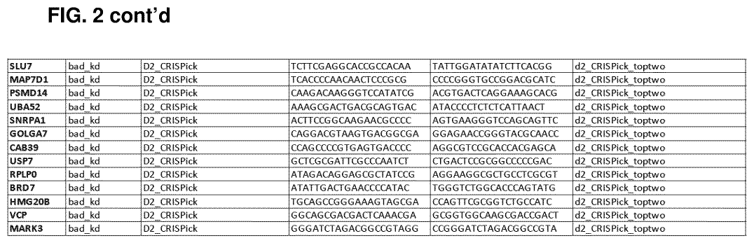

In total, 222 different dual guide vectors targeting 75 different genes were designed. Vectors were designed according to the 4 following categories as shown in Tables 1-4 and are shown in FIG. 2.

TABLE 1
Group A
Group A: Targeting Guides to Alternative Promoters
88 dual guide vectors targeting 22 genes

| Subgroup | Description | Purpose |
|---|---|---|
| A1 | CRISPRi v3 guide pairs | Compare experimental results |
| | | to published data |
| A2 | CRISPick guide pairs | Account for differences |
| | targeted to same region | arising from use of a different |
| | as A1 | guide prediction algorithm |
| A3 | CRISPick guide pairs | Test if transcriptional repression |
| | targeting alternative | is improved upon targeting |
| | promoter (using methods | to alternative promoter |
| | described herein) | |
| A4 | A1 + A3 | Test splitting guide pair |
| | A1: use top ranking | across the canonical |
| | CRISPRi v3 guide | and alternative promoters |
| | A3: use top CRISPick | |
| | guide in alternative | |
| | promoter window | |

TABLE 2
Group B
Group B: Splitting Guide Pairs Across Canonical Promoters
69 dual guide vectors targeting 23 genes

| Subgroup | Description | Purpose |
|---|---|---|
| B1 | CRISPRi v3 P1 | Compare experimental results |
| | (canonical promoter 1) | to published data |
| | guide pair | |
| B2 | CRISPRi v3 P2 | Compare experimental results |
| | (canonical promoter 2) | to published data |
| | guide pair | |
| B3 | B1 + B2 | Test if transcriptional |
| | B1: use top ranking | repression is improved |
| | CRISPRi v3 P1 guide | upon splitting a guide pair |
| | B2: use top ranking | across the two canonical |
| | CRISPRi v3 P2 guide | promoters |

TABLE 3
Group C
Group C: Control Experiment -
Guides with Strong Transcriptional Knockdown
28 dual guide vectors targeting 14 genes

| Subgroup | Description | Purpose |
|---|---|---|
| C1 | CRISPRi v3 guide pairs | Compare experimental results |
| | exhibiting strong | to published data |
| | transcriptional repression | |
| | in Replogle et al. | |
| C2 | CRISPick guide pairs | Account for differences |
| | targeted to same region | arising from use of a different |
| | as C1 | guide prediction algorithm |

TABLE 4
Group D
Group D: Control Experiment -
Guides with Weak Transcriptional Knockdown
32 dual guide vectors targeting 16 genes

| Subgroup | Description | Purpose |
|---|---|---|
| D1 | CRISPRi v3 guide pairs | Compare experimental results |
| | exhibiting weak | to published data |
| | transcriptional repression | |
| | in Replogle et al. | |
| D2 | CRISPick guide pairs | Account for differences |
| | targeted to same region | arising from use of a different |
| | as D1 | guide prediction algorithm |

Once guide vectors were designed, guide pairs were synthesized as an oligo pool using Agilent HiFi Oligo Synthesis technology, then PCR amplified and cloned into a lentivirus expression vector. Lentivirus was generated and transduced into dCas9-KRAB-KOX1 or dCas9-KRAB-ZIM3 K562 cell lines. Cells were cultured for 6 days, FACS sorted for dCas9 CRISPRi machinery and guide positive cells, then cultured for an additional day and run on the 10×3′ Single Cell w/Feature Barcoding Technology platform. In total, 26,397 cells (10,953 for KOX1 and 15,444 for ZIM3) passed QC and were assigned guide pairs.

### Example 3: Experimental Results

The experimental results suggest that the strategy for identifying alternative promoters and targeting CRISPRi guides to 1 or 2 of the most active promoters is successful in improving transcriptional repression over the present best-in-class CRISPRi v3 library. For analysis of the data, KOX1 and ZIM3 data were combined into a single analysis in order to increase cell numbers and statistical power. ZIM3 was observed to perform better, or equally as well as, KOX1 in all tested cases.

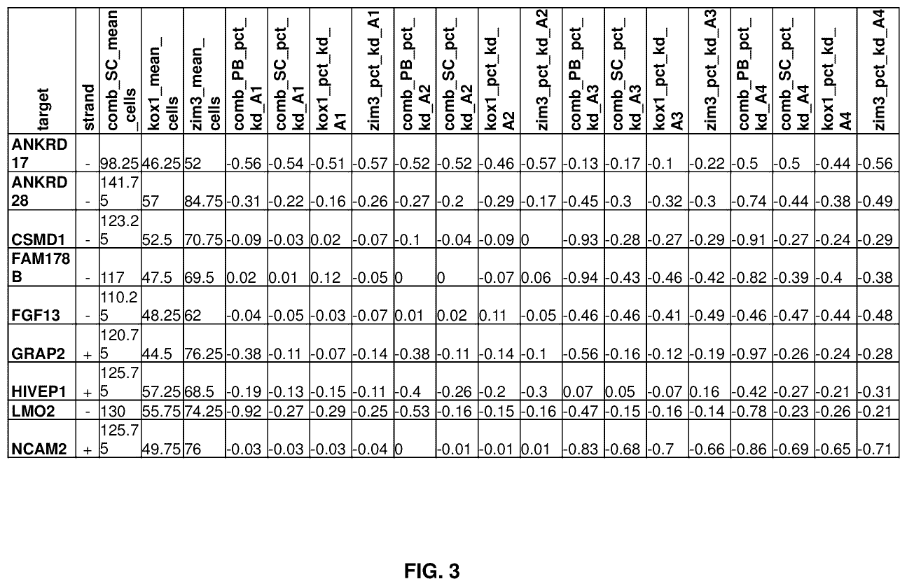

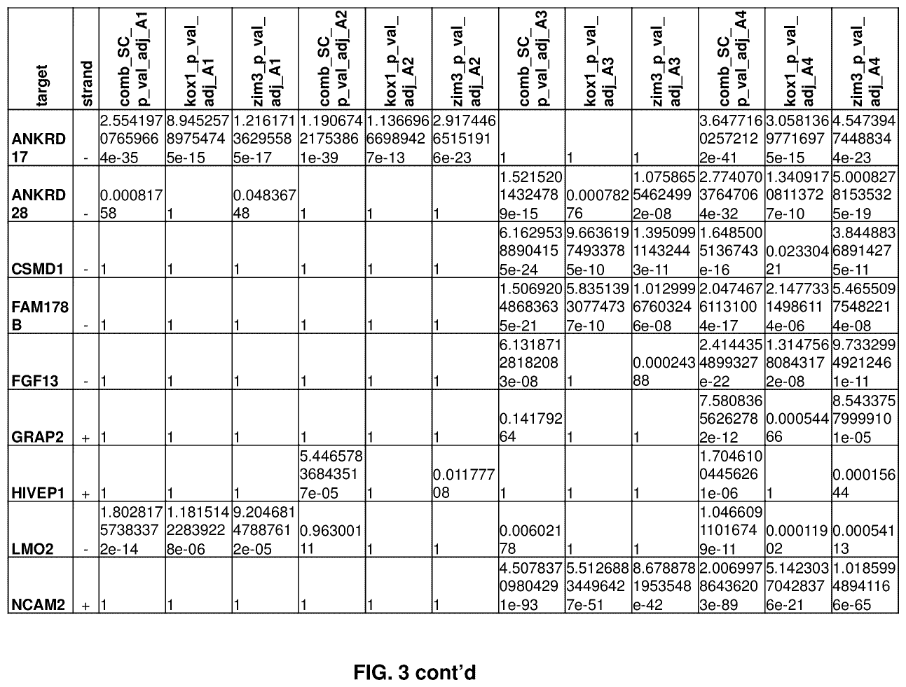

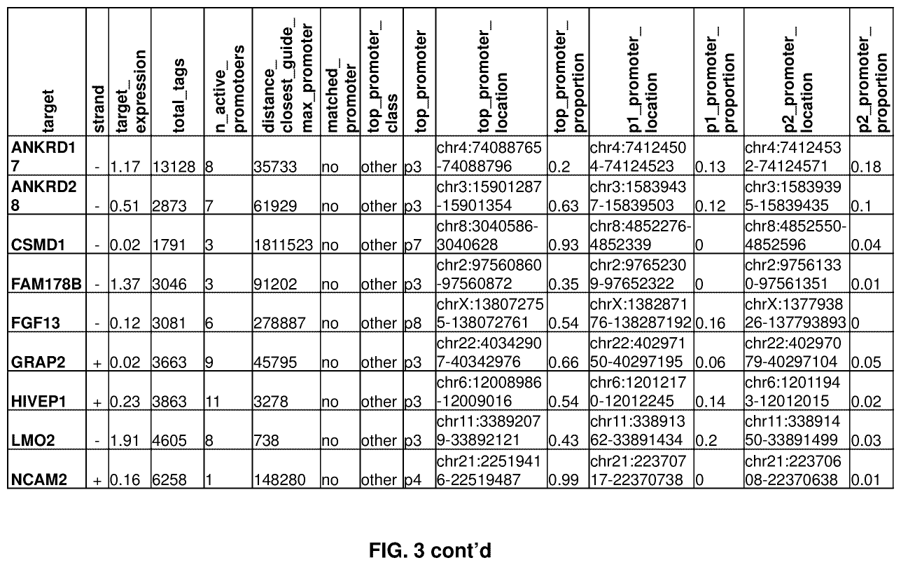

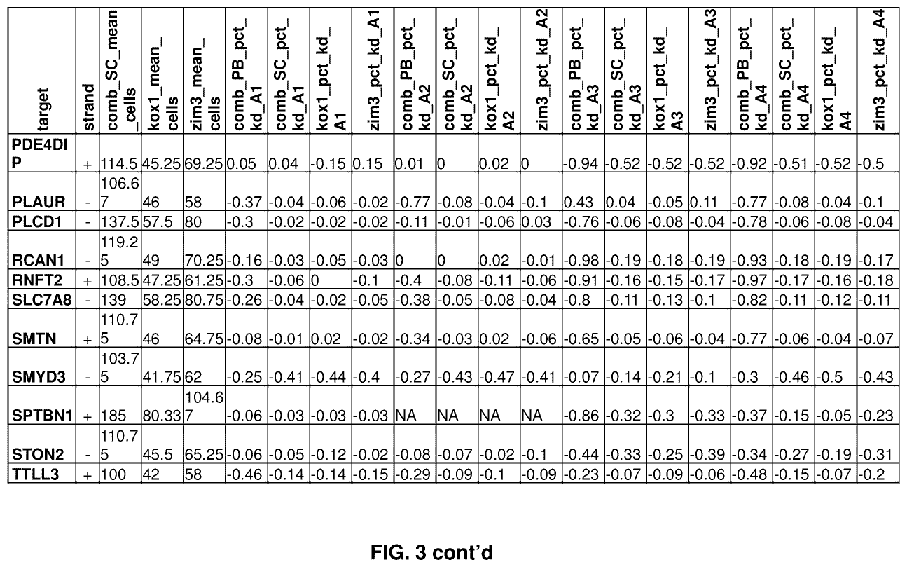

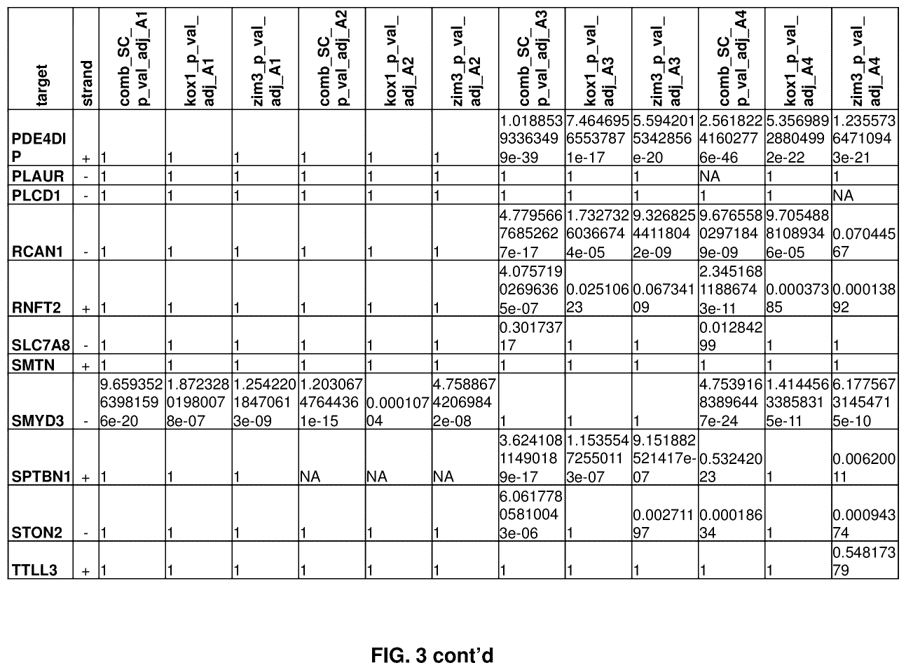

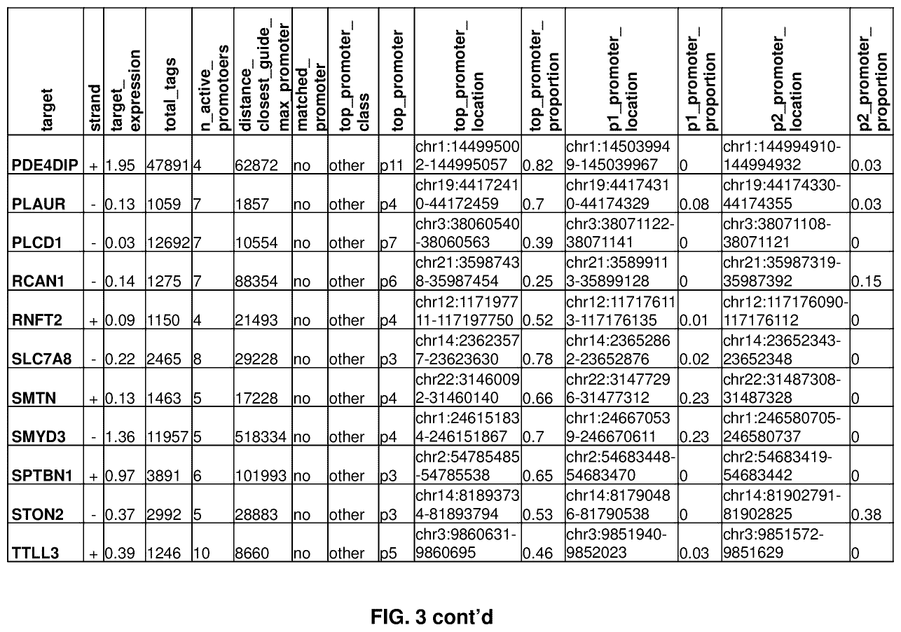

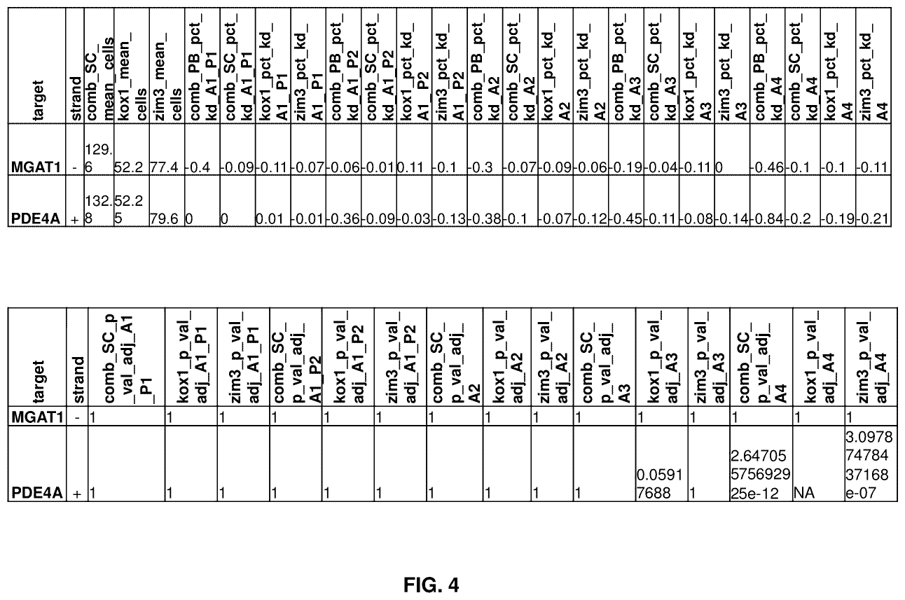

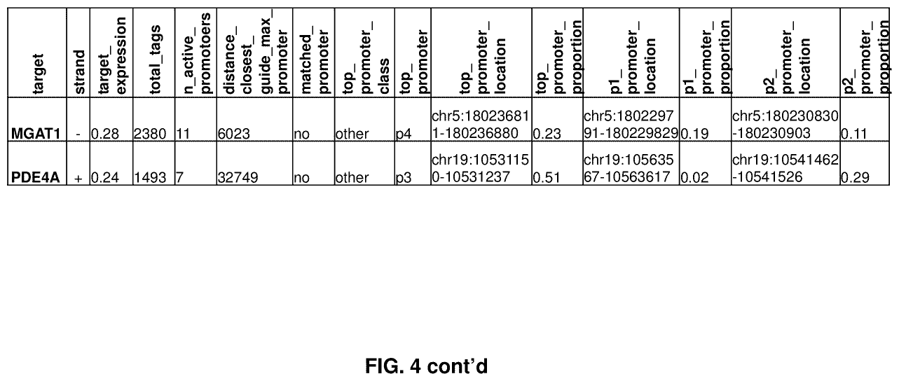

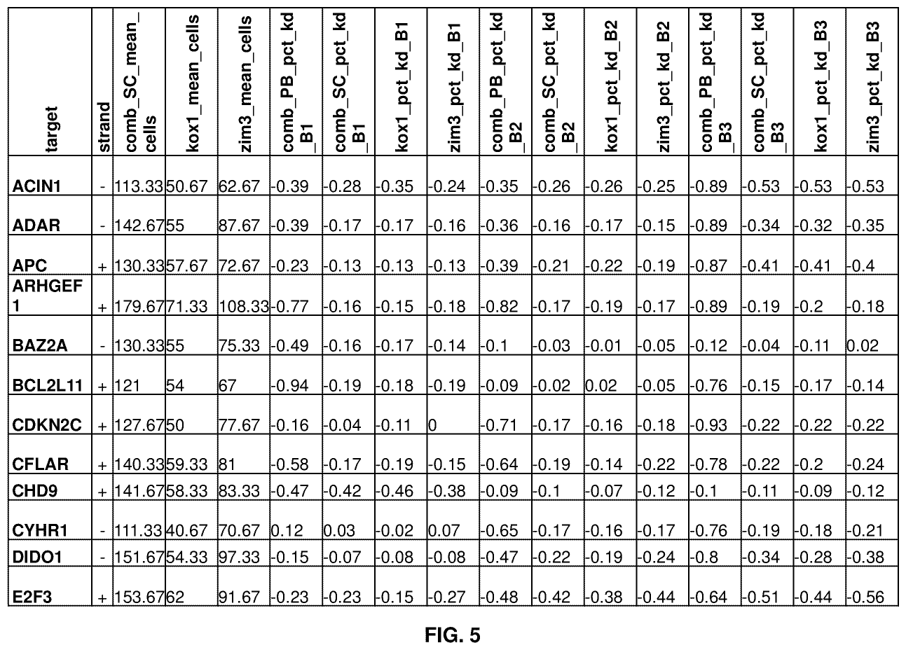

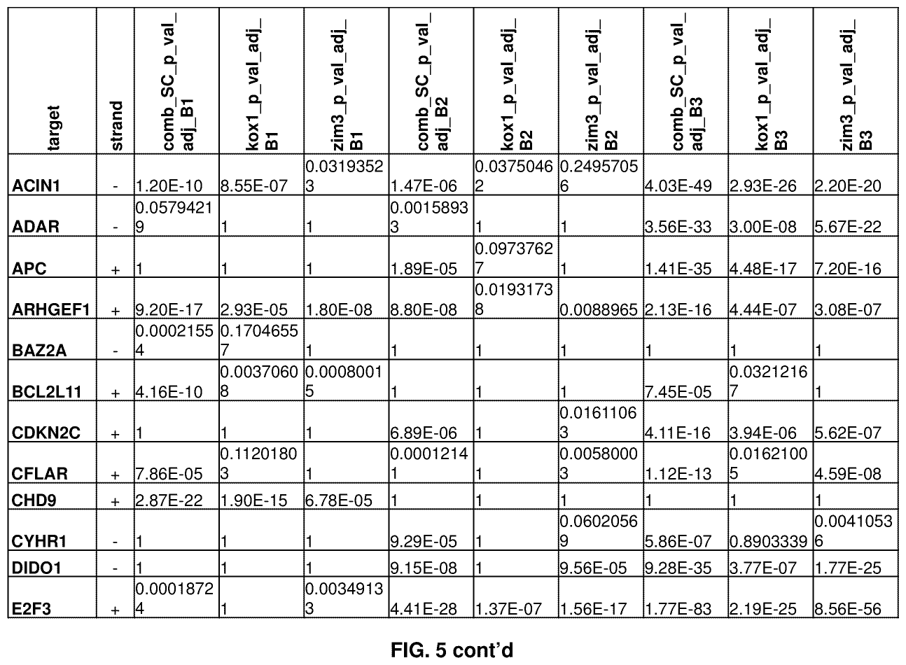

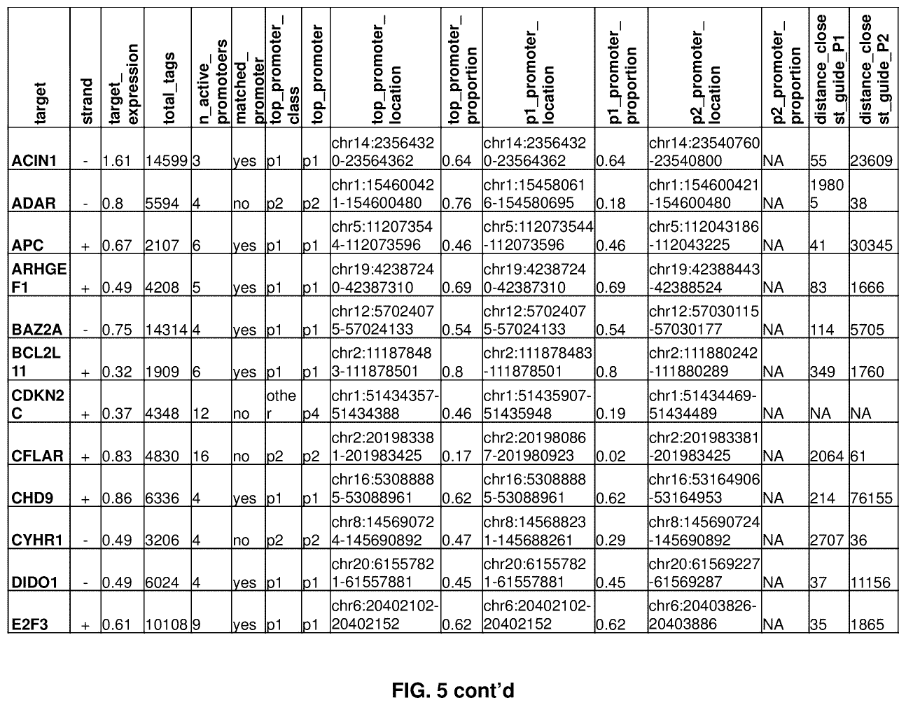

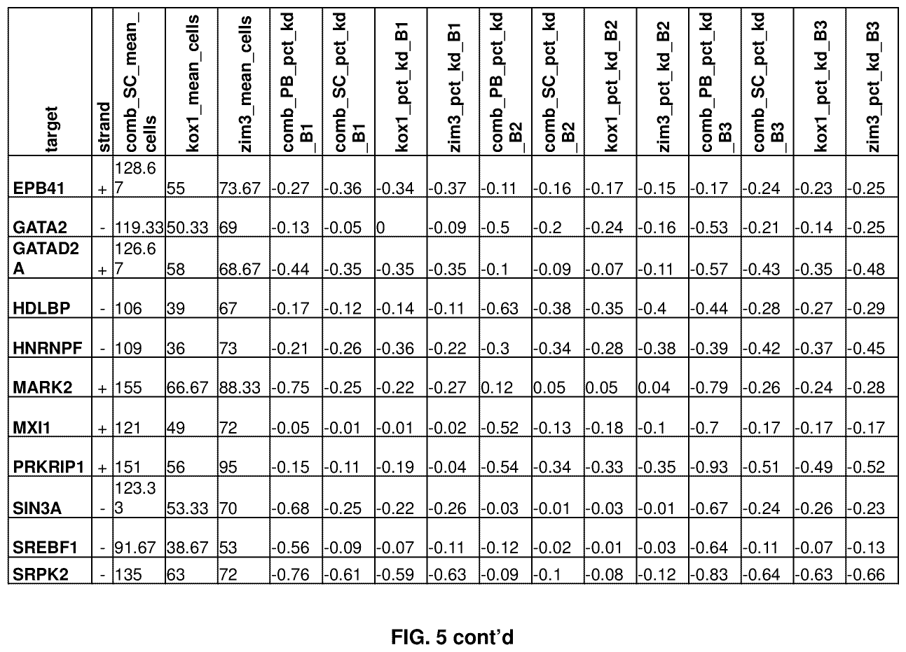

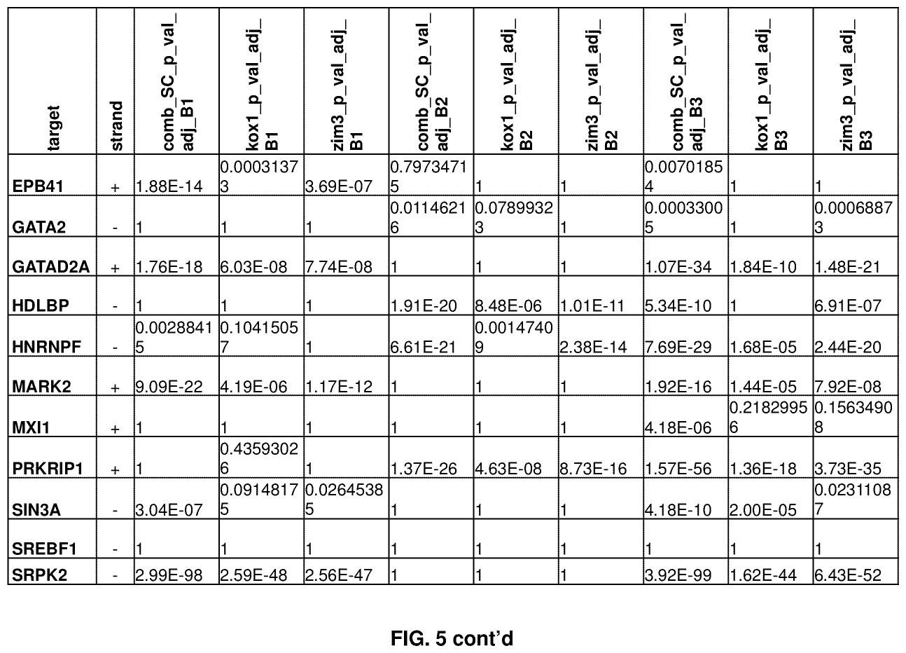

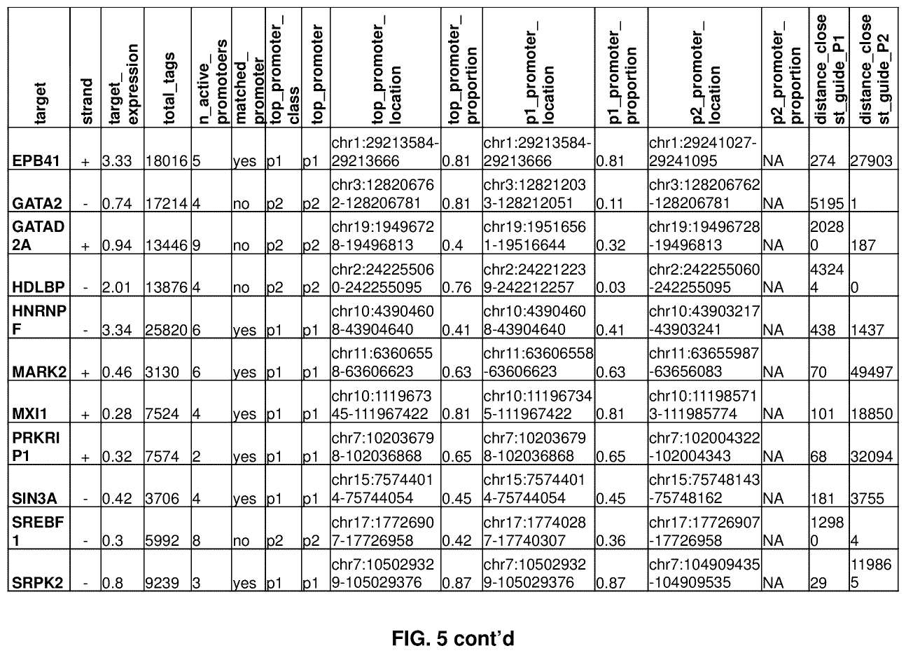

To quantify transcriptional repression for each guide pair, the percent knockdown of the target gene relative to a negative control as well as the significance value were calculated using a logistic regression-based differential expression analysis, using α<0.05 as our significance threshold. Percent knockdown and p-value data can be found in FIG. 3 (Group A; four guides), FIG. 4 (Group A; five guides), and FIG. 5 (Group B). Results are displayed in Tables 5 and 6.

TABLE 5
Results of Group A
Group A Results: Targeting Guides to Alternative Promoters

| Subgroup | Description | Results |
|---|---|---|
| A1 | CRISPRi v3 guide pairs | 4/22 genes differentially |
| | | expressed relative |
| | | to control |
| A2 | CRISPick guide pairs | 3/22 genes differentially |
| | targeted to same region | expressed relative |
| | as A1 | to control |
| A3 | CRISPick guide pairs | 11/22 genes differentially |
| | targeting alternative | expressed relative |
| | promoter | to control |
| A4 | A1 + A3 | 15/22 genes differentially |
| | A1: use top ranking | expressed relative |
| | CRISPRi v3 guide | to control |
| | A3: use top CRISPick | |
| | guide in alternative | |
| | promoter window | |

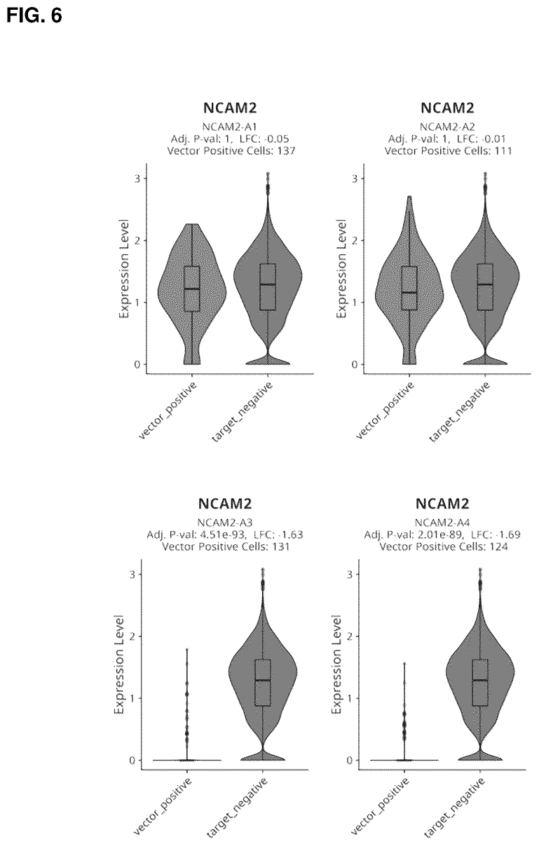

An example of data from Group A is shown in FIG. 6. NCAM2F expression levels for cells assigned an NCAM2-targeting guide (A1, A2, A3, or A4) are shown in red and labeled “vector_positive”, while NCAM2 expression levels in negative control cells are shown in blue and labeled “target_negative”. In this example, when A1 and A2 guide pairs targeting the canonical NCAM2 promoter as published in the CRISPRi v3 library are used, no significant changes in transcriptional expression are observed. However, when A3 and A4 guide pairs are used to target alternative promoters identified in K562 CAGE data, statistically significant transcriptional knockdown is observed. These results indicate that the approach of alternative promoter identification and CRISPRi guide design can increase transcriptional repression for genes that are minimally repressed by guides in the CRISPRi v3 library.

TABLE 6
Results of Group B
Group B Results: Splitting Guide Pairs Across Canonical Promoters

| Subgroup | Description | Results |
|---|---|---|
| B1 | CRISPRi v3 P1 | 13/23 genes differentially |
| | (canonical promoter 1) | expressed relative |
| | guide pair | to control |
| B2 | CRISPRi v3 P2 | 8/23 genes differentially |
| | (canonical promoter 2) | expressed relative |
| | guide pair | to control |
| B3 | B1 + B2 | 20/23 genes differentially |
| | B1: use top ranking | expressed relative |
| | CRISPRi v3 P1 guide | to control |
| | B2: use top ranking | |
| | CRISPRi v3 P2 guide | |

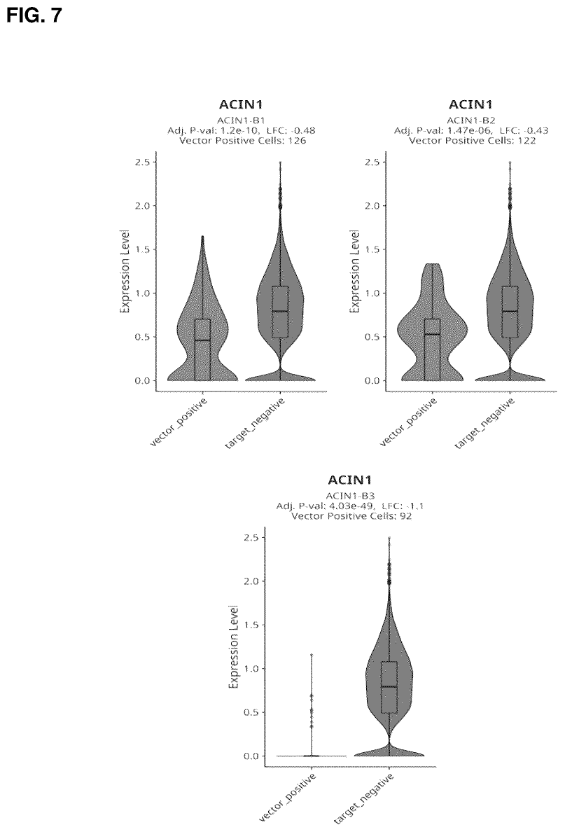

An example of data from Group B is shown in FIG. 7. ACIN1 expression levels for cells assigned an ACIN1-targeting guide (B1, B2, or B3) are shown in red and labeled “vector_positive”, while ACIN1 expression levels in negative control cells are shown in blue and labeled “target_negative”. In this example, when B1 and B2 guide pairs targeting either the P1 or P2 canonical promoter as published in the CRISPRi v3 library are used, statistically significant, but moderate transcriptional repression is observed. However, when B3 guide pairs are used, where guide pairs are split across the two promoters, statistically significant and more pronounced transcriptional repression is observed. These results demonstrated that in cases where more than one promoter is active for a given gene, targeting two promoters simultaneously can lead to increased transcriptional repression compared to targeting of a single promoter.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. Moreover, nothing disclosed herein is intended to be dedicated to the public regardless of whether such disclosure is explicitly recited in the claims.

The scope of the present invention, therefore, is not intended to be limited to the exemplary embodiments shown and described herein. Rather, the scope and spirit of present invention is embodied by the appended claims. In the claims, 35 U.S.C. § 112 (f) or 35 U.S.C. § 112 (6) is expressly defined as being invoked for a limitation in the claim only when the exact phrase “means for” or the exact phrase “step for” is recited at the beginning of such limitation in the claim; if such exact phrase is not used in a limitation in the claim, then 35 U.S.C. § 112 (f) or 35 U.S.C. § 112 (6) is not invoked.

1. A method of identifying promoters for targeted expression modulation, the method comprising:
(a) assessing, for a cell type of interest, using a computer:
(i) transcript start site (TSS) expression data, and
(ii) genome annotation data,
thereby generating quantitative data relating to active promoters and their relative genomic location;
(b) computer processing said quantitative data to identify, for each of a plurality of genes, the most highly utilized promoter that is responsible for at least a threshold percentage of transcription,
thereby identifying promoters for targeted expression modulation for the cell type of interest.

2. The method of claim 1, wherein at least one of said promoters for targeted modulation is an alternative promotor and this therefore not a primary promoter as identified by the FANTOM5 project or as targeted by CRISPRi v3 guide RNA library.

3. The method of claim 1, wherein said promoters for targeted modulation are alternative promotors and are therefore not primary promoters identified by the FANTOM5 project or targeted by CRISPRi v3 guide RNA library.

4. A method of identifying one or more target genes for alternative promoter targeting, the method comprising:
(a) assessing, for a cell type of interest, using a computer:
(i) transcript start site (TSS) expression data, and
(ii) genome annotation data,
thereby generating quantitative data relating to active promoters and their relative genomic location;
(b) computer processing said quantitative data to identify genes for which the most highly utilized promoter is responsible for at least a threshold percentage of transcription and is not the primary promoter of the target gene as identified by the FANTOM5 project or as targeted by CRISPRi v3 guide RNA library,
thereby identifying target genes for alternative promoter targeting.

5. The method of claim 1, wherein the genome annotation data comprises genomic location data for gene annotations and for known CRISPR-Cas guide RNA targets.

6. The method of claim 5, wherein the genome annotation data further comprises genomic location data for exon annotations.

7. The method of claim 5, wherein the most highly utilized promoter is at least a threshold distance away from the closest known CRISPR-Cas guide RNA target.

8. The method of claim 7, wherein said closest known CRISPR-Cas guide RNA is from CRISPRi v3 guide RNA library.

9. The method of claim 7, wherein the threshold distance away from the closest known CRISPR-Cas guide RNA target is 3 kilobases (kb).

10. (canceled)

11. The method of claim 1, wherein the threshold percentage of transcription is 40%.

12-13. (canceled)

14. The method of claim 1, wherein the TSS expression data comprises cap analysis of gene expression (CAGE) data.

15. (canceled)

16. The method of claim 1, wherein the quantitative data relating to active promoters and their relative genomic location includes expression peak data and the closest gene annotation, the closest exon annotation, and/or the closest CRISPR-Cas guide RNA target annotation for each expression peak.

17. The method of claim 1, wherein the computer processing comprises removing expression peaks for which the closest gene annotation is greater than 500 base pairs away.

18. The method of claim 1, wherein the computer processing comprises removing expression peaks that are within 10% of the 3′ end of a gene.

19. The method of claim 1, wherein the computer processing comprises ranking expression peaks for each gene based on the ratio of read counts for each expression peak to the total read counts for all expression peaks for a given gene. (i.e., the percentage of reads for each expression peak).

20. (canceled)

21. The method of claim 1, further comprising a step of designing CRISPR-Cas guide RNAs to target CRISPRi or CRISPRa effector polypeptide to alternative promoters of the identified target genes, and a step of producing the designed CRISPR-Cas guide RNAs.

22-26. (canceled)

27. The method of claim 1, wherein the cell type of interest is a mouse cell, a non-human primate cell, or a human cell.

28-29. (canceled)

30. A promoter-targeted CRISPR-Cas guide RNA library, comprising a plurality of CRISPR-Cas guide RNAs or nucleic acids encoding the plurality of CRISPR-Cas guide RNAs, wherein the CRISPR-Cas guide RNAs are targeted to said most highly utilized promoters of claim 1.

31. The promoter-targeted CRISPR-Cas guide RNA library of claim 30, wherein said library comprises at least one CRISPR-Cas guide RNA targeted to an alternative promoter, which is not a primary promoter identified by the FANTOM5 project or as targeted by CRISPRi v3 guide RNA library.

32. The promoter-targeted CRISPR-Cas guide RNA library of claim 30, wherein said library does not include CRISPR-Cas guide RNAs that are targeted to promoters identified by the FANTOM5 project or as targeted by CRISPRi v3 guide RNA library.

33-34. (canceled)

35. A method of modulating expression of a target gene, the method comprising introducing into a cell or expressing in the cell:
(a) one or more CRISPR-Cas guide RNAs targeted to an alternative promoter of a target gene; and
(b) one or more CRISPR-Cas guide RNAs targeted to a primary promoter of the target gene, wherein the primary promoter was identified by the FANTOM5 project or as targeted by CRISPRi v3 guide RNA library,
wherein the cell expresses a CRISPRi or CRISPRa effector polypeptide,
thereby modulating expression of the target gene.

36. (canceled)

37. The method of claim 35, wherein expression of two or more target genes is modulated by introducing CRISPR-Cas guide RNAs targeted to an alternative promoter and a primary promoter of each target gene.

38-41. (canceled)


SEQUENCE LISTING (444 sequences)

SEQ ID NO: 1 - RNA, 20 nt, synthetic construct
  source 1..20: mol_type = other RNA
gcgggacccgatgctatgag

SEQ ID NO: 2 - RNA, 20 nt, synthetic construct
  source 1..20: mol_type = other RNA
gcgagctccccagctggcgg

SEQ ID NO: 3 - RNA, 20 nt, synthetic construct
  source 1..20: mol_type = other RNA
gctgttccggagggtgcctg

SEQ ID NO: 4 - RNA, 20 nt, synthetic construct
  source 1..20: mol_type = other RNA
ggtgagagctggcggagcgg

SEQ ID NO: 5 - RNA, 20 nt, synthetic construct
  source 1..20: mol_type = other RNA
gtcccacagaagcattccaa

SEQ ID NO: 6 - RNA, 20 nt, synthetic construct
  source 1..20: mol_type = other RNA
ggaggaggaggcacagttaa

SEQ ID NO: 7 - RNA, 20 nt, synthetic construct
  source 1..20: mol_type = other RNA
gtctcccccggggcagagta

SEQ ID NO: 8 - RNA, 20 nt, synthetic construct
  source 1..20: mol_type = other RNA
ggcgcgcgagagggaccggg

SEQ ID NO: 9 - RNA, 20 nt, synthetic construct
  source 1..20: mol_type = other RNA
gggcgcgccgcagaaagggc

SEQ ID NO: 10 - RNA, 20 nt, synthetic construct
  source 1..20: mol_type = other RNA
ggaccccaggcggtgagggt

SEQ ID NO: 11 - RNA, 20 nt, synthetic construct
  source 1..20: mol_type = other RNA
ggctctagcagaggcggccg

SEQ ID NO: 12 - RNA, 20 nt, synthetic construct
  source 1..20: mol_type = other RNA
gagcctggcggcccaaatcc

SEQ ID NO: 13 - RNA, 20 nt, synthetic construct
  source 1..20: mol_type = other RNA
gctggggctgctgcacacag

SEQ ID NO: 14 - RNA, 20 nt, synthetic construct
  source 1..20: mol_type = other RNA
gggcgcgcagcggtacctag

SEQ ID NO: 15 - RNA, 20 nt, synthetic construct
  source 1..20: mol_type = other RNA
gcgggtgacccatgtcgcga

SEQ ID NO: 16 - RNA, 20 nt, synthetic construct
  source 1..20: mol_type = other RNA
gtgagtagcgacagcaccgg

SEQ ID NO: 17 - RNA, 20 nt, synthetic construct
  source 1..20: mol_type = other RNA
gttggaaaggaatctcacta

SEQ ID NO: 18 - RNA, 20 nt, synthetic construct
  source 1..20: mol_type = other RNA
ggctgcgcgggtgattggct

SEQ ID NO: 19 - RNA, 20 nt, synthetic construct
  source 1..20: mol_type = other RNA
gccggctgtgcagtagaacg

SEQ ID NO: 20 - RNA, 20 nt, synthetic construct
  source 1..20: mol_type = other RNA
gagctctccgcagaatccag

SEQ ID NO: 21 - RNA, 20 nt, synthetic construct
  source 1..20: mol_type = other RNA
gggacgcgggtagccgtctg

SEQ ID NO: 22 - RNA, 20 nt, synthetic construct
  source 1..20: mol_type = other RNA
gctcacaggacgggaccagt

SEQ ID NO: 23 - RNA, 20 nt, synthetic construct
  source 1..20: mol_type = other RNA
gtgcctggcagattaaccaa

SEQ ID NO: 24 - RNA, 20 nt, synthetic construct
  source 1..20: mol_type = other RNA
gtcccaggcacccacgccca

SEQ ID NO: 25 - RNA, 20 nt, synthetic construct
  source 1..20: mol_type = other RNA
ggagccaaataatcacccga

SEQ ID NO: 26 - RNA, 20 nt, synthetic construct
  source 1..20: mol_type = other RNA
gagttgcgtccctagatcca

SEQ ID NO: 27 - RNA, 20 nt, synthetic construct
  source 1..20: mol_type = other RNA
tctgacaaaggattcataac

SEQ ID NO: 28 - RNA, 20 nt, synthetic construct
  source 1..20: mol_type = other RNA
cagactcgcaccatcccacg

SEQ ID NO: 29 - RNA, 20 nt, synthetic construct
  source 1..20: mol_type = other RNA
aaatctgaaaaggataactg

SEQ ID NO: 30 - RNA, 20 nt, synthetic construct
  source 1..20: mol_type = other RNA
actggcttcagaagcatctc

SEQ ID NO: 31 - RNA, 20 nt, synthetic construct
  source 1..20: mol_type = other RNA
acaaacataacctcctccgg

SEQ ID NO: 32 - RNA, 20 nt, synthetic construct
  source 1..20: mol_type = other RNA
tgtttgtattgctaatcccc

SEQ ID NO: 33 - RNA, 20 nt, synthetic construct
  source 1..20: mol_type = other RNA
ccatgagtgctagcgagagg

SEQ ID NO: 34 - RNA, 20 nt, synthetic construct
  source 1..20: mol_type = other RNA
tgcacgccctgagccgagaa

SEQ ID NO: 35 - RNA, 20 nt, synthetic construct
  source 1..20: mol_type = other RNA
gagccacgaaccctagaccc

SEQ ID NO: 36 - RNA, 20 nt, synthetic construct
  source 1..20: mol_type = other RNA
aaggagcaagcaacttgtat

SEQ ID NO: 37 - RNA, 20 nt, synthetic construct
  source 1..20: mol_type = other RNA
gcagtggagagcgattgccc

SEQ ID NO: 38 - RNA, 20 nt, synthetic construct
  source 1..20: mol_type = other RNA
cacttgagtagcgacagcac

SEQ ID NO: 39 - RNA, 20 nt, synthetic construct
  source 1..20: mol_type = other RNA
cactaggggcttgactgcgt

SEQ ID NO: 40 - RNA, 20 nt, synthetic construct
  source 1..20: mol_type = other RNA
gagactacacagaccatgat

SEQ ID NO: 41 - RNA, 20 nt, synthetic construct
  source 1..20: mol_type = other RNA
agatctagagcaacaccgtc

SEQ ID NO: 42 - RNA, 20 nt, synthetic construct
  source 1..20: mol_type = other RNA
atggcttagacctctcgtat

SEQ ID NO: 43 - RNA, 20 nt, synthetic construct
  source 1..20: mol_type = other RNA
acacagctcgcgactcaggt

SEQ ID NO: 44 - RNA, 20 nt, synthetic construct
  source 1..20: mol_type = other RNA
gctcacaggacgggaccagt

SEQ ID NO: 45 - RNA, 20 nt, synthetic construct
  source 1..20: mol_type = other RNA
gacgcacccacagctatgta

SEQ ID NO: 46 - RNA, 20 nt, synthetic construct
  source 1..20: mol_type = other RNA
aggatataagaaaacagcgc

SEQ ID NO: 47 - RNA, 20 nt, synthetic construct
  source 1..20: mol_type = other RNA
caccgcacccggcctagaag

SEQ ID NO: 48 - RNA, 20 nt, synthetic construct
  source 1..20: mol_type = other RNA
aggcggaatgaagatgatgg

SEQ ID NO: 49 - RNA, 20 nt, synthetic construct
  source 1..20: mol_type = other RNA
aatcatggaggaaggcaagc

SEQ ID NO: 50 - RNA, 20 nt, synthetic construct
  source 1..20: mol_type = other RNA
ctagaagacattcttgaggt

SEQ ID NO: 51 - RNA, 20 nt, synthetic construct
  source 1..20: mol_type = other RNA
gcccccttcccgaaataccc

SEQ ID NO: 52 - RNA, 20 nt, synthetic construct
  source 1..20: mol_type = other RNA
tcagcagttcctaggagagt

SEQ ID NO: 53 - RNA, 20 nt, synthetic construct
  source 1..20: mol_type = other RNA
aagttaaacccaggaagctt

SEQ ID NO: 54 - RNA, 20 nt, synthetic construct
  source 1..20: mol_type = other RNA
tccgccaccagccgactgag

SEQ ID NO: 55 - RNA, 20 nt, synthetic construct
  source 1..20: mol_type = other RNA
tgtgtgtttacgaataaact

SEQ ID NO: 56 - RNA, 20 nt, synthetic construct
  source 1..20: mol_type = other RNA
gtcccactccggaactcata

SEQ ID NO: 57 - RNA, 20 nt, synthetic construct
  source 1..20: mol_type = other RNA
gctgcgggccaatcgccccc

SEQ ID NO: 58 - RNA, 20 nt, synthetic construct
  source 1..20: mol_type = other RNA
attccgcaaatatctccctg

SEQ ID NO: 59 - RNA, 20 nt, synthetic construct
  source 1..20: mol_type = other RNA
taaggtcaggaagagctaaa

SEQ ID NO: 60 - RNA, 20 nt, synthetic construct
  source 1..20: mol_type = other RNA
cttaccttcagcccctccac

SEQ ID NO: 61 - RNA, 20 nt, synthetic construct
  source 1..20: mol_type = other RNA
gattccgagggggttaacgg

SEQ ID NO: 62 - RNA, 20 nt, synthetic construct
  source 1..20: mol_type = other RNA
gggtaggagggaaggaccat

SEQ ID NO: 63 - RNA, 20 nt, synthetic construct
  source 1..20: mol_type = other RNA
ccctccagctggagcaaaag

SEQ ID NO: 64 - RNA, 20 nt, synthetic construct
  source 1..20: mol_type = other RNA
aataccatagatgtccacta

SEQ ID NO: 65 - RNA, 20 nt, synthetic construct
  source 1..20: mol_type = other RNA
aagatggttacatttcaagt

SEQ ID NO: 66 - RNA, 20 nt, synthetic construct
  source 1..20: mol_type = other RNA
ggccaatcctaagcatgcca

SEQ ID NO: 67 - RNA, 20 nt, synthetic construct
  source 1..20: mol_type = other RNA
atattctctaccctcttcac

SEQ ID NO: 68 - RNA, 20 nt, synthetic construct
  source 1..20: mol_type = other RNA
gtgaatcatacacagatgtg

SEQ ID NO: 69 - RNA, 20 nt, synthetic construct
  source 1..20: mol_type = other RNA
gcgggacccgatgctatgag

SEQ ID NO: 70 - RNA, 20 nt, synthetic construct
  source 1..20: mol_type = other RNA
gcgagctccccagctggcgg

SEQ ID NO: 71 - RNA, 20 nt, synthetic construct
  source 1..20: mol_type = other RNA
gctgttccggagggtgcctg

SEQ ID NO: 72 - RNA, 20 nt, synthetic construct
  source 1..20: mol_type = other RNA
ggaagggagccccccggcgg

SEQ ID NO: 73 - RNA, 20 nt, synthetic construct
  source 1..20: mol_type = other RNA
gtcccacagaagcattccaa

SEQ ID NO: 74 - RNA, 20 nt, synthetic construct
  source 1..20: mol_type = other RNA
ggaggaggaggcacagttaa

SEQ ID NO: 75 - RNA, 20 nt, synthetic construct
  source 1..20: mol_type = other RNA
gtctcccccggggcagagta

SEQ ID NO: 76 - RNA, 20 nt, synthetic construct
  source 1..20: mol_type = other RNA
gggagcgccggagcaagcgg

SEQ ID NO: 77 - RNA, 20 nt, synthetic construct
  source 1..20: mol_type = other RNA
gggcgcgccgcagaaagggc

SEQ ID NO: 78 - RNA, 20 nt, synthetic construct
  source 1..20: mol_type = other RNA
ggctctagcagaggcggccg

SEQ ID NO: 79 - RNA, 20 nt, synthetic construct
  source 1..20: mol_type = other RNA
gagcctggcggcccaaatcc

SEQ ID NO: 80 - RNA, 20 nt, synthetic construct
  source 1..20: mol_type = other RNA
gggcgcgcagcggtacctag

SEQ ID NO: 81 - RNA, 20 nt, synthetic construct
  source 1..20: mol_type = other RNA
gggagctgccctcgcgacat

SEQ ID NO: 82 - RNA, 20 nt, synthetic construct
  source 1..20: mol_type = other RNA
gtgagtagcgacagcaccgg

SEQ ID NO: 83 - RNA, 20 nt, synthetic construct
  source 1..20: mol_type = other RNA
gttggaaaggaatctcacta

SEQ ID NO: 84 - RNA, 20 nt, synthetic construct
  source 1..20: mol_type = other RNA
gcccaggctgcgcgggtgat

SEQ ID NO: 85 - RNA, 20 nt, synthetic construct
  source 1..20: mol_type = other RNA
gccggctgtgcagtagaacg

SEQ ID NO: 86 - RNA, 20 nt, synthetic construct
  source 1..20: mol_type = other RNA
gagctctccgcagaatccag

SEQ ID NO: 87 - RNA, 20 nt, synthetic construct
  source 1..20: mol_type = other RNA
gggacgcgggtagccgtctg

SEQ ID NO: 88 - RNA, 20 nt, synthetic construct
  source 1..20: mol_type = other RNA
ggctcacaggacgggaccag

SEQ ID NO: 89 - RNA, 20 nt, synthetic construct
  source 1..20: mol_type = other RNA
gtgcctggcagattaaccaa

SEQ ID NO: 90 - RNA, 20 nt, synthetic construct
  source 1..20: mol_type = other RNA
gtcccaggcacccacgccca

SEQ ID NO: 91 - RNA, 20 nt, synthetic construct
  source 1..20: mol_type = other RNA
ggccgggcccaagatggctc

SEQ ID NO: 92 - RNA, 20 nt, synthetic construct
  source 1..20: mol_type = other RNA
ggccatcatgcagcctggtt

SEQ ID NO: 93 - RNA, 20 nt, synthetic construct
  source 1..20: mol_type = other RNA
ggactcccggagctaggggt

SEQ ID NO: 94 - RNA, 20 nt, synthetic construct
  source 1..20: mol_type = other RNA
ggctctagtgggcgcttcac

SEQ ID NO: 95 - RNA, 20 nt, synthetic construct
  source 1..20: mol_type = other RNA
gacagcgaaaacaaagatgg

SEQ ID NO: 96 - RNA, 20 nt, synthetic construct
  source 1..20: mol_type = other RNA
gaatggctgcgagaccctag

SEQ ID NO: 97 - RNA, 20 nt, synthetic construct
  source 1..20: mol_type = other RNA
ggatgcaacggattgcgagg

SEQ ID NO: 98 - RNA, 20 nt, synthetic construct
  source 1..20: mol_type = other RNA
gttctggaattcgggaccgc

SEQ ID NO: 99 - RNA, 20 nt, synthetic construct
  source 1..20: mol_type = other RNA
gacttgagcagcgagtcccg

SEQ ID NO: 100 - RNA, 20 nt, synthetic construct
  source 1..20: mol_type = other RNA
gcgcccctcggccgcacaca

SEQ ID NO: 101 - RNA, 20 nt, synthetic construct
  source 1..20: mol_type = other RNA
gcggggggcgagtaagccag

SEQ ID NO: 102 - RNA, 20 nt, synthetic construct
  source 1..20: mol_type = other RNA
gccccaccttgaggaagacg

SEQ ID NO: 103 - RNA, 20 nt, synthetic construct
  source 1..20: mol_type = other RNA
gcagcccgccagggtgtcac

SEQ ID NO: 104 - RNA, 20 nt, synthetic construct
  source 1..20: mol_type = other RNA
gaagcgggagccgggaccca

SEQ ID NO: 105 - RNA, 20 nt, synthetic construct
  source 1..20: mol_type = other RNA
gatgttgggagcagcaggtc

SEQ ID NO: 106 - RNA, 20 nt, synthetic construct
  source 1..20: mol_type = other RNA
ggcaaggaggcgcgctgcga

SEQ ID NO: 107 - RNA, 20 nt, synthetic construct
  source 1..20: mol_type = other RNA
gtcgcaccgaggaggcggct

SEQ ID NO: 108 - RNA, 20 nt, synthetic construct
  source 1..20: mol_type = other RNA
gcgcgctccgggcgccagaa

SEQ ID NO: 109 - RNA, 20 nt, synthetic construct
  source 1..20: mol_type = other RNA
ggaaactccgaggggggaag

SEQ ID NO: 110 - RNA, 20 nt, synthetic construct
  source 1..20: mol_type = other RNA
gccaagaaagggcgccgagg

SEQ ID NO: 111 - RNA, 20 nt, synthetic construct
  source 1..20: mol_type = other RNA
ggacgctagcgcgggaacca

SEQ ID NO: 112 - RNA, 20 nt, synthetic construct
  source 1..20: mol_type = other RNA
gcggccggcggagcgagtgg

SEQ ID NO: 113 - RNA, 20 nt, synthetic construct
  source 1..20: mol_type = other RNA
gtcacccgcagcgcctgaag

SEQ ID NO: 114 - RNA, 20 nt, synthetic construct
  source 1..20: mol_type = other RNA
ggccggcgagagggaaacaa

SEQ ID NO: 115 - RNA, 20 nt, synthetic construct
  source 1..20: mol_type = other RNA
gaaccggcaccctctctgag

SEQ ID NO: 116 - RNA, 20 nt, synthetic construct
  source 1..20: mol_type = other RNA
ggaagcggttcagcaggcag

SEQ ID NO: 117 - RNA, 20 nt, synthetic construct
  source 1..20: mol_type = other RNA
gaggaagcccgggggcaccg

SEQ ID NO: 118 - RNA, 20 nt, synthetic construct
  source 1..20: mol_type = other RNA
gcgctcgctcccgcaactgg

SEQ ID NO: 119 - RNA, 20 nt, synthetic construct
  source 1..20: mol_type = other RNA
ggcgaccacgcactacgggt

SEQ ID NO: 120 - RNA, 20 nt, synthetic construct
  source 1..20: mol_type = other RNA
gccctggcctgggacggtcc

SEQ ID NO: 121 - RNA, 20 nt, synthetic construct
  source 1..20: mol_type = other RNA
ggactaaacgcaccagcgga

SEQ ID NO: 122 - RNA, 20 nt, synthetic construct
  source 1..20: mol_type = other RNA
gcttacagggtaggagctgg

SEQ ID NO: 123 - RNA, 20 nt, synthetic construct
  source 1..20: mol_type = other RNA
gcgccgcgggcgccacctga

SEQ ID NO: 124 - RNA, 20 nt, synthetic construct
  source 1..20: mol_type = other RNA
gtaggaactgtggcgaaaca

SEQ ID NO: 125 - RNA, 20 nt, synthetic construct
  source 1..20: mol_type = other RNA
gggagcaggagaccaataca

SEQ ID NO: 126 - RNA, 20 nt, synthetic construct
  source 1..20: mol_type = other RNA
ggcaggtcccccccgagcag

SEQ ID NO: 127 - RNA, 20 nt, synthetic construct
  source 1..20: mol_type = other RNA
gggcggctagagcgacgcgg

SEQ ID NO: 128 - RNA, 20 nt, synthetic construct
  source 1..20: mol_type = other RNA
gagcacaccagaagcaaggt

SEQ ID NO: 129 - RNA, 20 nt, synthetic construct
  source 1..20: mol_type = other RNA
gaggtaaacaaaccacgcgc

SEQ ID NO: 130 - RNA, 20 nt, synthetic construct
  source 1..20: mol_type = other RNA
ggaagaggcgcaggaagcca

SEQ ID NO: 131 - RNA, 20 nt, synthetic construct
  source 1..20: mol_type = other RNA
gtccctggctgactcagcag

SEQ ID NO: 132 - RNA, 20 nt, synthetic construct
  source 1..20: mol_type = other RNA
gctgggaccgagctgagggg

SEQ ID NO: 133 - RNA, 20 nt, synthetic construct
  source 1..20: mol_type = other RNA
gaggcggaggatgttcccgg

SEQ ID NO: 134 - RNA, 20 nt, synthetic construct
  source 1..20: mol_type = other RNA
gtaggcaggggtgctgaata

SEQ ID NO: 135 - RNA, 20 nt, synthetic construct
  source 1..20: mol_type = other RNA
ggattcgcagtacttctgtt

SEQ ID NO: 136 - RNA, 20 nt, synthetic construct
  source 1..20: mol_type = other RNA
gaggaagacgaagggagcta

SEQ ID NO: 137 - RNA, 20 nt, synthetic construct
  source 1..20: mol_type = other RNA
gcgcactggaagtggccccg

SEQ ID NO: 138 - RNA, 20 nt, synthetic construct
  source 1..20: mol_type = other RNA
ggcagcctggttaggagcaa

SEQ ID NO: 139 - RNA, 20 nt, synthetic construct
  source 1..20: mol_type = other RNA
gcccggagcgcagggctgtg

SEQ ID NO: 140 - RNA, 20 nt, synthetic construct
  source 1..20: mol_type = other RNA
gtatctctgcagaccgcaag

SEQ ID NO: 141 - RNA, 20 nt, synthetic construct
  source 1..20: mol_type = other RNA
gacagcgaaaacaaagatgg

SEQ ID NO: 142 - RNA, 20 nt, synthetic construct
  source 1..20: mol_type = other RNA
gaatggctgcgagaccctag

SEQ ID NO: 143 - RNA, 20 nt, synthetic construct
  source 1..20: mol_type = other RNA
ggcggccggcagctgaaaaa

SEQ ID NO: 144 - RNA, 20 nt, synthetic construct
  source 1..20: mol_type = other RNA
gttctggaattcgggaccgc

SEQ ID NO: 145 - RNA, 20 nt, synthetic construct
  source 1..20: mol_type = other RNA
gggccctgctaggatggatg

SEQ ID NO: 146 - RNA, 20 nt, synthetic construct
  source 1..20: mol_type = other RNA
gcggggcaaggaactttcca

SEQ ID NO: 147 - RNA, 20 nt, synthetic construct
  source 1..20: mol_type = other RNA
gcggggggcgagtaagccag

SEQ ID NO: 148 - RNA, 20 nt, synthetic construct
  source 1..20: mol_type = other RNA
gccccaccttgaggaagacg

SEQ ID NO: 149 - RNA, 20 nt, synthetic construct
  source 1..20: mol_type = other RNA
gcagcccgccagggtgtcac

SEQ ID NO: 150 - RNA, 20 nt, synthetic construct
  source 1..20: mol_type = other RNA
gaagcgggagccgggaccca

SEQ ID NO: 151 - RNA, 20 nt, synthetic construct
  source 1..20: mol_type = other RNA
gatgttgggagcagcaggtc

SEQ ID NO: 152 - RNA, 20 nt, synthetic construct
  source 1..20: mol_type = other RNA
ggaggctcgggaagtcaggc

SEQ ID NO: 153 - RNA, 20 nt, synthetic construct
  source 1..20: mol_type = other RNA
gtcgcaccgaggaggcggct

SEQ ID NO: 154 - RNA, 20 nt, synthetic construct
  source 1..20: mol_type = other RNA
gcgcgctccgggcgccagaa

SEQ ID NO: 155 - RNA, 20 nt, synthetic construct
  source 1..20: mol_type = other RNA
ggtgcccagactgggaagga

SEQ ID NO: 156 - RNA, 20 nt, synthetic construct
  source 1..20: mol_type = other RNA
gcgcctggtctgcagtttgt

SEQ ID NO: 157 - RNA, 20 nt, synthetic construct
  source 1..20: mol_type = other RNA
ggacgctagcgcgggaacca

SEQ ID NO: 158 - RNA, 20 nt, synthetic construct
  source 1..20: mol_type = other RNA
gcggccggcggagcgagtgg

SEQ ID NO: 159 - RNA, 20 nt, synthetic construct
  source 1..20: mol_type = other RNA
gtcacccgcagcgcctgaag

SEQ ID NO: 160 - RNA, 20 nt, synthetic construct
  source 1..20: mol_type = other RNA
gccgcacgcggaggatccta

SEQ ID NO: 161 - RNA, 20 nt, synthetic construct
  source 1..20: mol_type = other RNA
gcggcctcttgcgctcctag

SEQ ID NO: 162 - RNA, 20 nt, synthetic construct
  source 1..20: mol_type = other RNA
gactccccggactccagcag

SEQ ID NO: 163 - RNA, 20 nt, synthetic construct
  source 1..20: mol_type = other RNA
gtcagcagagaaaccggcgt

SEQ ID NO: 164 - RNA, 20 nt, synthetic construct
  source 1..20: mol_type = other RNA
gaccggcagctgaacccacc

SEQ ID NO: 165 - RNA, 20 nt, synthetic construct
  source 1..20: mol_type = other RNA
ggcggaggtgcaggtcgaag

SEQ ID NO: 166 - RNA, 20 nt, synthetic construct
  source 1..20: mol_type = other RNA
gcctgcctctggtcggttcc

SEQ ID NO: 167 - RNA, 20 nt, synthetic construct
  source 1..20: mol_type = other RNA
gagcttgaacaggtaatcgt

SEQ ID NO: 168 - RNA, 20 nt, synthetic construct
  source 1..20: mol_type = other RNA
gaagctgcccgggaaagtgg

SEQ ID NO: 169 - RNA, 20 nt, synthetic construct
  source 1..20: mol_type = other RNA
ggtcgataagctggaactga

SEQ ID NO: 170 - RNA, 20 nt, synthetic construct
  source 1..20: mol_type = other RNA
ggccccggcgcggagcaggc

SEQ ID NO: 171 - RNA, 20 nt, synthetic construct
  source 1..20: mol_type = other RNA
gcgcgggcgaggggaactgg

SEQ ID NO: 172 - RNA, 20 nt, synthetic construct
  source 1..20: mol_type = other RNA
gctcctttggagaaaaagag

SEQ ID NO: 173 - RNA, 20 nt, synthetic construct
  source 1..20: mol_type = other RNA
gtccgctgcgcctacctcag

SEQ ID NO: 174 - RNA, 20 nt, synthetic construct
  source 1..20: mol_type = other RNA
ggactaaggcgacgcaactc

SEQ ID NO: 175 - RNA, 20 nt, synthetic construct
  source 1..20: mol_type = other RNA
gcaagccgtactagcacgga

SEQ ID NO: 176 - RNA, 20 nt, synthetic construct
  source 1..20: mol_type = other RNA
cataacacgccctaacgagc

SEQ ID NO: 177 - RNA, 20 nt, synthetic construct
  source 1..20: mol_type = other RNA
ataatgacagcaactcaacc

SEQ ID NO: 178 - RNA, 20 nt, synthetic construct
  source 1..20: mol_type = other RNA
caacgttctaaattccacga

SEQ ID NO: 179 - RNA, 20 nt, synthetic construct
  source 1..20: mol_type = other RNA
gagcgccaggaaccgaccag

SEQ ID NO: 180 - RNA, 20 nt, synthetic construct
  source 1..20: mol_type = other RNA
acttcgcttagtgtaaccag

SEQ ID NO: 181 - RNA, 20 nt, synthetic construct
  source 1..20: mol_type = other RNA
gctcccaccaccaccacagt

SEQ ID NO: 182 - RNA, 20 nt, synthetic construct
  source 1..20: mol_type = other RNA
tttggatgtgcagtcctata

SEQ ID NO: 183 - RNA, 20 nt, synthetic construct
  source 1..20: mol_type = other RNA
tgttacctgccactgaatca

SEQ ID NO: 184 - RNA, 20 nt, synthetic construct
  source 1..20: mol_type = other RNA
gtctaggtgaccttccccgg

SEQ ID NO: 185 - RNA, 20 nt, synthetic construct
  source 1..20: mol_type = other RNA
agggaggcgatcgataactc

SEQ ID NO: 186 - RNA, 20 nt, synthetic construct
  source 1..20: mol_type = other RNA
taattattcaaagctcacaa

SEQ ID NO: 187 - RNA, 20 nt, synthetic construct
  source 1..20: mol_type = other RNA
gaaccacccgaggccgtgat

SEQ ID NO: 188 - RNA, 20 nt, synthetic construct
  source 1..20: mol_type = other RNA
ggggcccgccgcccgcaccc

SEQ ID NO: 189 - RNA, 20 nt, synthetic construct
  source 1..20: mol_type = other RNA
gaggagcgggcggaagacaa

SEQ ID NO: 190 - RNA, 20 nt, synthetic construct
  source 1..20: mol_type = other RNA
gtgcggcaggacgtaagtga

SEQ ID NO: 191 - RNA, 20 nt, synthetic construct
  source 1..20: mol_type = other RNA
gctccgggcccggcatcccg

SEQ ID NO: 192 - RNA, 20 nt, synthetic construct
  source 1..20: mol_type = other RNA
gtgcagccggtgaagaaacg

SEQ ID NO: 193 - RNA, 20 nt, synthetic construct
  source 1..20: mol_type = other RNA
gccacgcggaggcgagatgg

SEQ ID NO: 194 - RNA, 20 nt, synthetic construct
  source 1..20: mol_type = other RNA
ggtctgtagtttccgatgtg

SEQ ID NO: 195 - RNA, 20 nt, synthetic construct
  source 1..20: mol_type = other RNA
gggcagcagaggaagccgag

SEQ ID NO: 196 - RNA, 20 nt, synthetic construct
  source 1..20: mol_type = other RNA
gggaccccccgcccaaagtc

SEQ ID NO: 197 - RNA, 20 nt, synthetic construct
  source 1..20: mol_type = other RNA
gcagcggcgcacacccgacg

SEQ ID NO: 198 - RNA, 20 nt, synthetic construct
  source 1..20: mol_type = other RNA
gaggtccggaaagagagaac

SEQ ID NO: 199 - RNA, 20 nt, synthetic construct
  source 1..20: mol_type = other RNA
gagcacacgaaccttccacg

SEQ ID NO: 200 - RNA, 20 nt, synthetic construct
  source 1..20: mol_type = other RNA
gtcccaggaagcagccgcgg

SEQ ID NO: 201 - RNA, 20 nt, synthetic construct
  source 1..20: mol_type = other RNA
gattctgtcggatggacttg

SEQ ID NO: 202 - RNA, 20 nt, synthetic construct
  source 1..20: mol_type = other RNA
gctcgctggcgggaggccac

SEQ ID NO: 203 - RNA, 20 nt, synthetic construct
  source 1..20: mol_type = other RNA
gacaggactgagactcccct

SEQ ID NO: 204 - RNA, 20 nt, synthetic construct
  source 1..20: mol_type = other RNA
ggcgaggcggccgagctggt

SEQ ID NO: 205 - RNA, 20 nt, synthetic construct
  source 1..20: mol_type = other RNA
gagcgaggagagcaagcggg

SEQ ID NO: 206 - RNA, 20 nt, synthetic construct
  source 1..20: mol_type = other RNA
gcgcgaggtggcagtggcag

SEQ ID NO: 207 - RNA, 20 nt, synthetic construct
  source 1..20: mol_type = other RNA
tgcagacgccggatccatga

SEQ ID NO: 208 - RNA, 20 nt, synthetic construct
  source 1..20: mol_type = other RNA
cccgcatgacggcctctagg

SEQ ID NO: 209 - RNA, 20 nt, synthetic construct
  source 1..20: mol_type = other RNA
actgactagccgggcgataa

SEQ ID NO: 210 - RNA, 20 nt, synthetic construct
  source 1..20: mol_type = other RNA
tcttcgaggcaccgccacaa

SEQ ID NO: 211 - RNA, 20 nt, synthetic construct
  source 1..20: mol_type = other RNA
tcaccccaacaactcccgcg

SEQ ID NO: 212 - RNA, 20 nt, synthetic construct
  source 1..20: mol_type = other RNA
caagacaagggtccatatcg

SEQ ID NO: 213 - RNA, 20 nt, synthetic construct
  source 1..20: mol_type = other RNA
aaagcgactgacgcagtgac

SEQ ID NO: 214 - RNA, 20 nt, synthetic construct
  source 1..20: mol_type = other RNA
acttccggcaagaacgcccc

SEQ ID NO: 215 - RNA, 20 nt, synthetic construct
  source 1..20: mol_type = other RNA
caggacgtaagtgacggcga

SEQ ID NO: 216 - RNA, 20 nt, synthetic construct
  source 1..20: mol_type = other RNA
ccagccccgtgagtgacccc

SEQ ID NO: 217 - RNA, 20 nt, synthetic construct
  source 1..20: mol_type = other RNA
gctcgcgattcgcccaatct

SEQ ID NO: 218 - RNA, 20 nt, synthetic construct
  source 1..20: mol_type = other RNA
atagacaggagcgctatccg

SEQ ID NO: 219 - RNA, 20 nt, synthetic construct
  source 1..20: mol_type = other RNA
atattgactgaaccccatac

SEQ ID NO: 220 - RNA, 20 nt, synthetic construct
  source 1..20: mol_type = other RNA
tgcagccgggaaagtagcga

SEQ ID NO: 221 - RNA, 20 nt, synthetic construct
  source 1..20: mol_type = other RNA
ggcagcgacgactcaaacga

SEQ ID NO: 222 - RNA, 20 nt, synthetic construct
  source 1..20: mol_type = other RNA
gggatctagacggccgtagg

SEQ ID NO: 223 - RNA, 20 nt, synthetic construct
  source 1..20: mol_type = other RNA
gacccacagagaggggaccg

SEQ ID NO: 224 - RNA, 20 nt, synthetic construct
  source 1..20: mol_type = other RNA
gagggctccggggtgaggga

SEQ ID NO: 225 - RNA, 20 nt, synthetic construct
  source 1..20: mol_type = other RNA
gttccggagggtgcctgcgg

SEQ ID NO: 226 - RNA, 20 nt, synthetic construct
  source 1..20: mol_type = other RNA
ggaagggagccccccggcgg

SEQ ID NO: 227 - RNA, 20 nt, synthetic construct
  source 1..20: mol_type = other RNA
gtccgttggaatgcttctgt

SEQ ID NO: 228 - RNA, 20 nt, synthetic construct
  source 1..20: mol_type = other RNA
gcagactgcagggctgacta

SEQ ID NO: 229 - RNA, 20 nt, synthetic construct
  source 1..20: mol_type = other RNA
ggtgccctactctgccccgg

SEQ ID NO: 230 - RNA, 20 nt, synthetic construct
  source 1..20: mol_type = other RNA
gggagcgccggagcaagcgg

SEQ ID NO: 231 - RNA, 20 nt, synthetic construct
  source 1..20: mol_type = other RNA
gggcgagtcgaaaggcgagg

SEQ ID NO: 232 - RNA, 20 nt, synthetic construct
  source 1..20: mol_type = other RNA
gatgggaccccaggcggtga

SEQ ID NO: 233 - RNA, 20 nt, synthetic construct
  source 1..20: mol_type = other RNA
gccgctgcctggatatagtg

SEQ ID NO: 234 - RNA, 20 nt, synthetic construct
  source 1..20: mol_type = other RNA
gcagttgcagctccgggatg

SEQ ID NO: 235 - RNA, 20 nt, synthetic construct
  source 1..20: mol_type = other RNA
gctggcgggccatgtaacca

SEQ ID NO: 236 - RNA, 20 nt, synthetic construct
  source 1..20: mol_type = other RNA
ggagccggaagcccgctgac

SEQ ID NO: 237 - RNA, 20 nt, synthetic construct
  source 1..20: mol_type = other RNA
gggagctgccctcgcgacat

SEQ ID NO: 238 - RNA, 20 nt, synthetic construct
  source 1..20: mol_type = other RNA
ggctactcaagtgagtcccg

SEQ ID NO: 239 - RNA, 20 nt, synthetic construct
  source 1..20: mol_type = other RNA
gactaggggcttgactgcgt

SEQ ID NO: 240 - RNA, 20 nt, synthetic construct
  source 1..20: mol_type = other RNA
gcccaggctgcgcgggtgat

SEQ ID NO: 241 - RNA, 20 nt, synthetic construct
  source 1..20: mol_type = other RNA
ggccactctcccgaaaccag

SEQ ID NO: 242 - RNA, 20 nt, synthetic construct
  source 1..20: mol_type = other RNA
gctccgcagaatccagggga

SEQ ID NO: 243 - RNA, 20 nt, synthetic construct
  source 1..20: mol_type = other RNA
gcacagctcgcgactcaggt

SEQ ID NO: 244 - RNA, 20 nt, synthetic construct
  source 1..20: mol_type = other RNA
gggaggcagccccagtgagt

SEQ ID NO: 245 - RNA, 20 nt, synthetic construct
  source 1..20: mol_type = other RNA
ggattaaccaacggcaagag

SEQ ID NO: 246 - RNA, 20 nt, synthetic construct
  source 1..20: mol_type = other RNA
gccgagggggtgggtatccg

SEQ ID NO: 247 - RNA, 20 nt, synthetic construct
  source 1..20: mol_type = other RNA
cgggacccgatgctatgaga

SEQ ID NO: 248 - RNA, 20 nt, synthetic construct
  source 1..20: mol_type = other RNA
tagatccagggccgccaacg

SEQ ID NO: 249 - RNA, 20 nt, synthetic construct
  source 1..20: mol_type = other RNA
ggcaagaacagtgaaatctg

SEQ ID NO: 250 - RNA, 20 nt, synthetic construct
  source 1..20: mol_type = other RNA
gctacgctctaccgcgactt

SEQ ID NO: 251 - RNA, 20 nt, synthetic construct
  source 1..20: mol_type = other RNA
agaatcctagtagaacaagc

SEQ ID NO: 252 - RNA, 20 nt, synthetic construct
  source 1..20: mol_type = other RNA
ggtaaaattccacttcacca

SEQ ID NO: 253 - RNA, 20 nt, synthetic construct
  source 1..20: mol_type = other RNA
gcagccgccacgctgaaggc

SEQ ID NO: 254 - RNA, 20 nt, synthetic construct
  source 1..20: mol_type = other RNA
ggacgggcggaaaatccact

SEQ ID NO: 255 - RNA, 20 nt, synthetic construct
  source 1..20: mol_type = other RNA
cccctctcgctagcactcat

SEQ ID NO: 256 - RNA, 20 nt, synthetic construct
  source 1..20: mol_type = other RNA
gatgctgatactgatactgc

SEQ ID NO: 257 - RNA, 20 nt, synthetic construct
  source 1..20: mol_type = other RNA
tgccctggagacctctgata

SEQ ID NO: 258 - RNA, 20 nt, synthetic construct
  source 1..20: mol_type = other RNA
agcatatgaatgcttggagt

SEQ ID NO: 259 - RNA, 20 nt, synthetic construct
  source 1..20: mol_type = other RNA
gtggcggaggaggttcgtac

SEQ ID NO: 260 - RNA, 20 nt, synthetic construct
  source 1..20: mol_type = other RNA
ttgtccgagatctagcccgg

SEQ ID NO: 261 - RNA, 20 nt, synthetic construct
  source 1..20: mol_type = other RNA
ttggaaaggaatctcactag

SEQ ID NO: 262 - RNA, 20 nt, synthetic construct
  source 1..20: mol_type = other RNA
tgttcccgaagtgcacctgc

SEQ ID NO: 263 - RNA, 20 nt, synthetic construct
  source 1..20: mol_type = other RNA
atttaaatgtatgtctcccc

SEQ ID NO: 264 - RNA, 20 nt, synthetic construct
  source 1..20: mol_type = other RNA
catggcttagacctctcgta

SEQ ID NO: 265 - RNA, 20 nt, synthetic construct
  source 1..20: mol_type = other RNA
tgtacgccaagggatccgag

SEQ ID NO: 266 - RNA, 20 nt, synthetic construct
  source 1..20: mol_type = other RNA
gggaggcagccccagtgagt

SEQ ID NO: 267 - RNA, 20 nt, synthetic construct
  source 1..20: mol_type = other RNA
agtagctggactacatgcac

SEQ ID NO: 268 - RNA, 20 nt, synthetic construct
  source 1..20: mol_type = other RNA
ccttggaggatcctcgccag

SEQ ID NO: 269 - RNA, 20 nt, synthetic construct
  source 1..20: mol_type = other RNA
atgtttaaaaagcctcttct

SEQ ID NO: 270 - RNA, 20 nt, synthetic construct
  source 1..20: mol_type = other RNA
gatgatggtggtcttgctca

SEQ ID NO: 271 - RNA, 20 nt, synthetic construct
  source 1..20: mol_type = other RNA
gagtaaagatggagaaacac

SEQ ID NO: 272 - RNA, 20 nt, synthetic construct
  source 1..20: mol_type = other RNA
ataaaacatggtggagacag

SEQ ID NO: 273 - RNA, 20 nt, synthetic construct
  source 1..20: mol_type = other RNA
cgggtagggcaggcagcaga

SEQ ID NO: 274 - RNA, 20 nt, synthetic construct
  source 1..20: mol_type = other RNA
tgattaccaggaagcaccat

SEQ ID NO: 275 - RNA, 20 nt, synthetic construct
  source 1..20: mol_type = other RNA
taaaattaaaagttaaaccc

SEQ ID NO: 276 - RNA, 20 nt, synthetic construct
  source 1..20: mol_type = other RNA
tggctgggagggaccacgcg

SEQ ID NO: 277 - RNA, 20 nt, synthetic construct
  source 1..20: mol_type = other RNA
gtgtttacgaataaacttgg

SEQ ID NO: 278 - RNA, 20 nt, synthetic construct
  source 1..20: mol_type = other RNA
agccgtatgagttccggagt

SEQ ID NO: 279 - RNA, 20 nt, synthetic construct
  source 1..20: mol_type = other RNA
tggcgccccggggacagacc

SEQ ID NO: 280 - RNA, 20 nt, synthetic construct
  source 1..20: mol_type = other RNA
ttacctcagggagatatttg

SEQ ID NO: 281 - RNA, 20 nt, synthetic construct
  source 1..20: mol_type = other RNA
ggtcaggaagagctaaaggg

SEQ ID NO: 282 - RNA, 20 nt, synthetic construct
  source 1..20: mol_type = other RNA
tggcaggccataccttctca

SEQ ID NO: 283 - RNA, 20 nt, synthetic construct
  source 1..20: mol_type = other RNA
gaccggagcgcgtgaggctc

SEQ ID NO: 284 - RNA, 20 nt, synthetic construct
  source 1..20: mol_type = other RNA
ctagcctcaccccaacccga

SEQ ID NO: 285 - RNA, 20 nt, synthetic construct
  source 1..20: mol_type = other RNA
tgtgggagctggccagagcg

SEQ ID NO: 286 - RNA, 20 nt, synthetic construct
  source 1..20: mol_type = other RNA
atctccatagtggacatcta

SEQ ID NO: 287 - RNA, 20 nt, synthetic construct
  source 1..20: mol_type = other RNA
tgatggtgctcataagcaaa

SEQ ID NO: 288 - RNA, 20 nt, synthetic construct
  source 1..20: mol_type = other RNA
gagaagcagagaggaggcgc

SEQ ID NO: 289 - RNA, 20 nt, synthetic construct
  source 1..20: mol_type = other RNA
gaaaatggcaaccaaatgac

SEQ ID NO: 290 - RNA, 20 nt, synthetic construct
  source 1..20: mol_type = other RNA
atggatggctggtggatgga

SEQ ID NO: 291 - RNA, 20 nt, synthetic construct
  source 1..20: mol_type = other RNA
caccgcacccggcctagaag

SEQ ID NO: 292 - RNA, 20 nt, synthetic construct
  source 1..20: mol_type = other RNA
aggcggaatgaagatgatgg

SEQ ID NO: 293 - RNA, 20 nt, synthetic construct
  source 1..20: mol_type = other RNA
aatcatggaggaaggcaagc

SEQ ID NO: 294 - RNA, 20 nt, synthetic construct
  source 1..20: mol_type = other RNA
ctagaagacattcttgaggt

SEQ ID NO: 295 - RNA, 20 nt, synthetic construct
  source 1..20: mol_type = other RNA
gcccccttcccgaaataccc

SEQ ID NO: 296 - RNA, 20 nt, synthetic construct
  source 1..20: mol_type = other RNA
tcagcagttcctaggagagt

SEQ ID NO: 297 - RNA, 20 nt, synthetic construct
  source 1..20: mol_type = other RNA
aagttaaacccaggaagctt

SEQ ID NO: 298 - RNA, 20 nt, synthetic construct
  source 1..20: mol_type = other RNA
tccgccaccagccgactgag

SEQ ID NO: 299 - RNA, 20 nt, synthetic construct
  source 1..20: mol_type = other RNA
tgtgtgtttacgaataaact

SEQ ID NO: 300 - RNA, 20 nt, synthetic construct
  source 1..20: mol_type = other RNA
gtcccactccggaactcata

SEQ ID NO: 301 - RNA, 20 nt, synthetic construct
  source 1..20: mol_type = other RNA
gctgcgggccaatcgccccc

SEQ ID NO: 302 - RNA, 20 nt, synthetic construct
  source 1..20: mol_type = other RNA
attccgcaaatatctccctg

SEQ ID NO: 303 - RNA, 20 nt, synthetic construct
  source 1..20: mol_type = other RNA
taaggtcaggaagagctaaa

SEQ ID NO: 304 - RNA, 20 nt, synthetic construct
  source 1..20: mol_type = other RNA
cttaccttcagcccctccac

SEQ ID NO: 305 - RNA, 20 nt, synthetic construct
  source 1..20: mol_type = other RNA
gattccgagggggttaacgg

SEQ ID NO: 306 - RNA, 20 nt, synthetic construct
  source 1..20: mol_type = other RNA
gggtaggagggaaggaccat

SEQ ID NO: 307 - RNA, 20 nt, synthetic construct
  source 1..20: mol_type = other RNA
ccctccagctggagcaaaag

SEQ ID NO: 308 - RNA, 20 nt, synthetic construct
  source 1..20: mol_type = other RNA
aataccatagatgtccacta

SEQ ID NO: 309 - RNA, 20 nt, synthetic construct
  source 1..20: mol_type = other RNA
aagatggttacatttcaagt

SEQ ID NO: 310 - RNA, 20 nt, synthetic construct
  source 1..20: mol_type = other RNA
ggccaatcctaagcatgcca

SEQ ID NO: 311 - RNA, 20 nt, synthetic construct
  source 1..20: mol_type = other RNA
atattctctaccctcttcac

SEQ ID NO: 312 - RNA, 20 nt, synthetic construct
  source 1..20: mol_type = other RNA
gtgaatcatacacagatgtg

SEQ ID NO: 313 - RNA, 20 nt, synthetic construct
  source 1..20: mol_type = other RNA
gcgcactggaagtggccccg

SEQ ID NO: 314 - RNA, 20 nt, synthetic construct
  source 1..20: mol_type = other RNA
ggcagcctggttaggagcaa

SEQ ID NO: 315 - RNA, 20 nt, synthetic construct
  source 1..20: mol_type = other RNA
gcccggagcgcagggctgtg

SEQ ID NO: 316 - RNA, 20 nt, synthetic construct
  source 1..20: mol_type = other RNA
gtatctctgcagaccgcaag

SEQ ID NO: 317 - RNA, 20 nt, synthetic construct
  source 1..20: mol_type = other RNA
gcgcacagcgaaaacaaaga

SEQ ID NO: 318 - RNA, 20 nt, synthetic construct
  source 1..20: mol_type = other RNA
ggggcggacggccaccgaga

SEQ ID NO: 319 - RNA, 20 nt, synthetic construct
  source 1..20: mol_type = other RNA
ggcggccggcagctgaaaaa

SEQ ID NO: 320 - RNA, 20 nt, synthetic construct
  source 1..20: mol_type = other RNA
ggcggcgcggagccagaacg

SEQ ID NO: 321 - RNA, 20 nt, synthetic construct
  source 1..20: mol_type = other RNA
gggccctgctaggatggatg

SEQ ID NO: 322 - RNA, 20 nt, synthetic construct
  source 1..20: mol_type = other RNA
gcggggcaaggaactttcca

SEQ ID NO: 323 - RNA, 20 nt, synthetic construct
  source 1..20: mol_type = other RNA
ggtgttgcgcgctcactcgt

SEQ ID NO: 324 - RNA, 20 nt, synthetic construct
  source 1..20: mol_type = other RNA
gcgtccttccggctccgtcg

SEQ ID NO: 325 - RNA, 20 nt, synthetic construct
  source 1..20: mol_type = other RNA
ggtgctgccggactcggaaa

SEQ ID NO: 326 - RNA, 20 nt, synthetic construct
  source 1..20: mol_type = other RNA
gggatcgccgagcccgacct

SEQ ID NO: 327 - RNA, 20 nt, synthetic construct
  source 1..20: mol_type = other RNA
ggcagtgccgctgagggcag

SEQ ID NO: 328 - RNA, 20 nt, synthetic construct
  source 1..20: mol_type = other RNA
ggaggctcgggaagtcaggc

SEQ ID NO: 329 - RNA, 20 nt, synthetic construct
  source 1..20: mol_type = other RNA
ggctagcccagccgcctcct

SEQ ID NO: 330 - RNA, 20 nt, synthetic construct
  source 1..20: mol_type = other RNA
gggagccccgctttcatccg

SEQ ID NO: 331 - RNA, 20 nt, synthetic construct
  source 1..20: mol_type = other RNA
ggtgcccagactgggaagga

SEQ ID NO: 332 - RNA, 20 nt, synthetic construct
  source 1..20: mol_type = other RNA
gcgcctggtctgcagtttgt

SEQ ID NO: 333 - RNA, 20 nt, synthetic construct
  source 1..20: mol_type = other RNA
gacacgaacgcgcggagcgg

SEQ ID NO: 334 - RNA, 20 nt, synthetic construct
  source 1..20: mol_type = other RNA
gtcccggccggcggagcgag

SEQ ID NO: 335 - RNA, 20 nt, synthetic construct
  source 1..20: mol_type = other RNA
gagcgcgagcccaaatggga

SEQ ID NO: 336 - RNA, 20 nt, synthetic construct
  source 1..20: mol_type = other RNA
gggtagagaaggctacgtgg

SEQ ID NO: 337 - RNA, 20 nt, synthetic construct
  source 1..20: mol_type = other RNA
ggacacgcaagtgcctgtag

SEQ ID NO: 338 - RNA, 20 nt, synthetic construct
  source 1..20: mol_type = other RNA
gaggcgtgaagcggttcagc

SEQ ID NO: 339 - RNA, 20 nt, synthetic construct
  source 1..20: mol_type = other RNA
gctgccccgcgctgacaggg

SEQ ID NO: 340 - RNA, 20 nt, synthetic construct
  source 1..20: mol_type = other RNA
gctgcgcggtcccaacgcag

SEQ ID NO: 341 - RNA, 20 nt, synthetic construct
  source 1..20: mol_type = other RNA
gatggctgctgcggaggcgt

SEQ ID NO: 342 - RNA, 20 nt, synthetic construct
  source 1..20: mol_type = other RNA
gggagggccgaacagaccgg

SEQ ID NO: 343 - RNA, 20 nt, synthetic construct
  source 1..20: mol_type = other RNA
ggactgactaaacgcaccag

SEQ ID NO: 344 - RNA, 20 nt, synthetic construct
  source 1..20: mol_type = other RNA
ggaggctggcggggcttaca

SEQ ID NO: 345 - RNA, 20 nt, synthetic construct
  source 1..20: mol_type = other RNA
gcggaggcgactccctcagg

SEQ ID NO: 346 - RNA, 20 nt, synthetic construct
  source 1..20: mol_type = other RNA
ggggagtgcggagcgatcac

SEQ ID NO: 347 - RNA, 20 nt, synthetic construct
  source 1..20: mol_type = other RNA
gcggagcgcggtccctgtat

SEQ ID NO: 348 - RNA, 20 nt, synthetic construct
  source 1..20: mol_type = other RNA
gaccgtagggtcccgagcag

SEQ ID NO: 349 - RNA, 20 nt, synthetic construct
  source 1..20: mol_type = other RNA
ggcgacgcggcggcaggggt

SEQ ID NO: 350 - RNA, 20 nt, synthetic construct
  source 1..20: mol_type = other RNA
gggacagcacaccagaagca

SEQ ID NO: 351 - RNA, 20 nt, synthetic construct
  source 1..20: mol_type = other RNA
gacccgcggtgcccatggag

SEQ ID NO: 352 - RNA, 20 nt, synthetic construct
  source 1..20: mol_type = other RNA
gctcgggagcagaacagctt

SEQ ID NO: 353 - RNA, 20 nt, synthetic construct
  source 1..20: mol_type = other RNA
gcacagcctgaactgctgta

SEQ ID NO: 354 - RNA, 20 nt, synthetic construct
  source 1..20: mol_type = other RNA
gcccccaaacgcctgagatt

SEQ ID NO: 355 - RNA, 20 nt, synthetic construct
  source 1..20: mol_type = other RNA
gacgctgcgctctgaaggga

SEQ ID NO: 356 - RNA, 20 nt, synthetic construct
  source 1..20: mol_type = other RNA
ggcgccttgacaggtgaagt

SEQ ID NO: 357 - RNA, 20 nt, synthetic construct
  source 1..20: mol_type = other RNA
gtaaagggaaaaagctccag

SEQ ID NO: 358 - RNA, 20 nt, synthetic construct
  source 1..20: mol_type = other RNA
gctctattgtattggcggag

SEQ ID NO: 359 - RNA, 20 nt, synthetic construct
  source 1..20: mol_type = other RNA
gggtagagaaggctacgtgg

SEQ ID NO: 360 - RNA, 20 nt, synthetic construct
  source 1..20: mol_type = other RNA
gaaccggcaccctctctgag

SEQ ID NO: 361 - RNA, 20 nt, synthetic construct
  source 1..20: mol_type = other RNA
ggaagcggttcagcaggcag

SEQ ID NO: 362 - RNA, 20 nt, synthetic construct
  source 1..20: mol_type = other RNA
gaggaagcccgggggcaccg

SEQ ID NO: 363 - RNA, 20 nt, synthetic construct
  source 1..20: mol_type = other RNA
gctgcgcggtcccaacgcag

SEQ ID NO: 364 - RNA, 20 nt, synthetic construct
  source 1..20: mol_type = other RNA
ggcgaccacgcactacgggt

SEQ ID NO: 365 - RNA, 20 nt, synthetic construct
  source 1..20: mol_type = other RNA
gccctggcctgggacggtcc

SEQ ID NO: 366 - RNA, 20 nt, synthetic construct
  source 1..20: mol_type = other RNA
ggactgactaaacgcaccag

SEQ ID NO: 367 - RNA, 20 nt, synthetic construct
  source 1..20: mol_type = other RNA
gcttacagggtaggagctgg

SEQ ID NO: 368 - RNA, 20 nt, synthetic construct
  source 1..20: mol_type = other RNA
gcggaggcgactccctcagg

SEQ ID NO: 369 - RNA, 20 nt, synthetic construct
  source 1..20: mol_type = other RNA
gtaggaactgtggcgaaaca

SEQ ID NO: 370 - RNA, 20 nt, synthetic construct
  source 1..20: mol_type = other RNA
gggagcaggagaccaataca

SEQ ID NO: 371 - RNA, 20 nt, synthetic construct
  source 1..20: mol_type = other RNA
gaccgtagggtcccgagcag

SEQ ID NO: 372 - RNA, 20 nt, synthetic construct
  source 1..20: mol_type = other RNA
gggcggctagagcgacgcgg

SEQ ID NO: 373 - RNA, 20 nt, synthetic construct
  source 1..20: mol_type = other RNA
gagcacaccagaagcaaggt

SEQ ID NO: 374 - RNA, 20 nt, synthetic construct
  source 1..20: mol_type = other RNA
gacccgcggtgcccatggag

SEQ ID NO: 375 - RNA, 20 nt, synthetic construct
  source 1..20: mol_type = other RNA
ggaagaggcgcaggaagcca

SEQ ID NO: 376 - RNA, 20 nt, synthetic construct
  source 1..20: mol_type = other RNA
gcacagcctgaactgctgta

SEQ ID NO: 377 - RNA, 20 nt, synthetic construct
  source 1..20: mol_type = other RNA
gcccccaaacgcctgagatt

SEQ ID NO: 378 - RNA, 20 nt, synthetic construct
  source 1..20: mol_type = other RNA
gaggcggaggatgttcccgg

SEQ ID NO: 379 - RNA, 20 nt, synthetic construct
  source 1..20: mol_type = other RNA
ggcgccttgacaggtgaagt

SEQ ID NO: 380 - RNA, 20 nt, synthetic construct
  source 1..20: mol_type = other RNA
gtaaagggaaaaagctccag

SEQ ID NO: 381 - RNA, 20 nt, synthetic construct
  source 1..20: mol_type = other RNA
gctctattgtattggcggag

SEQ ID NO: 382 - RNA, 20 nt, synthetic construct
  source 1..20: mol_type = other RNA
ggcgaggcccagtgagttag

SEQ ID NO: 383 - RNA, 20 nt, synthetic construct
  source 1..20: mol_type = other RNA
gagcaaatcgcggaccaccg

SEQ ID NO: 384 - RNA, 20 nt, synthetic construct
  source 1..20: mol_type = other RNA
gaggcgacgcaactccggcc

SEQ ID NO: 385 - RNA, 20 nt, synthetic construct
  source 1..20: mol_type = other RNA
gtggcccgccaagttcaaac

SEQ ID NO: 386 - RNA, 20 nt, synthetic construct
  source 1..20: mol_type = other RNA
gaagtcccgtggcgccgggt

SEQ ID NO: 387 - RNA, 20 nt, synthetic construct
  source 1..20: mol_type = other RNA
gcgcgcgcggtagtaacccg

SEQ ID NO: 388 - RNA, 20 nt, synthetic construct
  source 1..20: mol_type = other RNA
gagcgccaggaaccgaccag

SEQ ID NO: 389 - RNA, 20 nt, synthetic construct
  source 1..20: mol_type = other RNA
gtcggggagagagtgtaata

SEQ ID NO: 390 - RNA, 20 nt, synthetic construct
  source 1..20: mol_type = other RNA
ggagggaggagatcggcgga

SEQ ID NO: 391 - RNA, 20 nt, synthetic construct
  source 1..20: mol_type = other RNA
gcggtttagacaccagacgg

SEQ ID NO: 392 - RNA, 20 nt, synthetic construct
  source 1..20: mol_type = other RNA
gaacctggcccgtgcggaaa

SEQ ID NO: 393 - RNA, 20 nt, synthetic construct
  source 1..20: mol_type = other RNA
gagaagcgcagcgacggcgt

SEQ ID NO: 394 - RNA, 20 nt, synthetic construct
  source 1..20: mol_type = other RNA
gatctccacaagcactcctt

SEQ ID NO: 395 - RNA, 20 nt, synthetic construct
  source 1..20: mol_type = other RNA
gctcgctcggctccgctccc

SEQ ID NO: 396 - RNA, 20 nt, synthetic construct
  source 1..20: mol_type = other RNA
aggtagcaccgaatctaccg

SEQ ID NO: 397 - RNA, 20 nt, synthetic construct
  source 1..20: mol_type = other RNA
ggagaggccaatcgtagaca

SEQ ID NO: 398 - RNA, 20 nt, synthetic construct
  source 1..20: mol_type = other RNA
cagcttgaacaggtaatcgt

SEQ ID NO: 399 - RNA, 20 nt, synthetic construct
  source 1..20: mol_type = other RNA
ggtattcagaattcgagcgc

SEQ ID NO: 400 - RNA, 20 nt, synthetic construct
  source 1..20: mol_type = other RNA
gttgtcactatatatctagg

SEQ ID NO: 401 - RNA, 20 nt, synthetic construct
  source 1..20: mol_type = other RNA
attctcagtacatactctta

SEQ ID NO: 402 - RNA, 20 nt, synthetic construct
  source 1..20: mol_type = other RNA
cacacacctccactatcacc

SEQ ID NO: 403 - RNA, 20 nt, synthetic construct
  source 1..20: mol_type = other RNA
gctatctgattcccattccc

SEQ ID NO: 404 - RNA, 20 nt, synthetic construct
  source 1..20: mol_type = other RNA
gagcatagagaggctaaatc

SEQ ID NO: 405 - RNA, 20 nt, synthetic construct
  source 1..20: mol_type = other RNA
aggaccccgccagtggcgtg

SEQ ID NO: 406 - RNA, 20 nt, synthetic construct
  source 1..20: mol_type = other RNA
gccgccacatcctctgaggt

SEQ ID NO: 407 - RNA, 20 nt, synthetic construct
  source 1..20: mol_type = other RNA
aaaggagataacgtactgtt

SEQ ID NO: 408 - RNA, 20 nt, synthetic construct
  source 1..20: mol_type = other RNA
atggagcccctaaatatgct

SEQ ID NO: 409 - RNA, 20 nt, synthetic construct
  source 1..20: mol_type = other RNA
gtttatccaacgagcaatct

SEQ ID NO: 410 - RNA, 20 nt, synthetic construct
  source 1..20: mol_type = other RNA
gcccctcctcaccctcgtag

SEQ ID NO: 411 - RNA, 20 nt, synthetic construct
  source 1..20: mol_type = other RNA
gcgggcggaagacaacggag

SEQ ID NO: 412 - RNA, 20 nt, synthetic construct
  source 1..20: mol_type = other RNA
gacgtaagtgacggcgaagg

SEQ ID NO: 413 - RNA, 20 nt, synthetic construct
  source 1..20: mol_type = other RNA
ggcgggagggactgggtcag

SEQ ID NO: 414 - RNA, 20 nt, synthetic construct
  source 1..20: mol_type = other RNA
ggccagccgcgtttcttcac

SEQ ID NO: 415 - RNA, 20 nt, synthetic construct
  source 1..20: mol_type = other RNA
gtatgccgggagccacgcgg

SEQ ID NO: 416 - RNA, 20 nt, synthetic construct
  source 1..20: mol_type = other RNA
gtctacaccgagctcctgag

SEQ ID NO: 417 - RNA, 20 nt, synthetic construct
  source 1..20: mol_type = other RNA
gcggccatcttggctccgtg

SEQ ID NO: 418 - RNA, 20 nt, synthetic construct
  source 1..20: mol_type = other RNA
gtctgctaccgtgactaaga

SEQ ID NO: 419 - RNA, 20 nt, synthetic construct
  source 1..20: mol_type = other RNA
ggagcaggcgaagagaatga

SEQ ID NO: 420 - RNA, 20 nt, synthetic construct
  source 1..20: mol_type = other RNA
gcaaagcgagctcaccatga

SEQ ID NO: 421 - RNA, 20 nt, synthetic construct
  source 1..20: mol_type = other RNA
gccgcggtcccgggcctaag

SEQ ID NO: 422 - RNA, 20 nt, synthetic construct
  source 1..20: mol_type = other RNA
ggcagccgcgggggtttcag

SEQ ID NO: 423 - RNA, 20 nt, synthetic construct
  source 1..20: mol_type = other RNA
gagattggcttggattctgt

SEQ ID NO: 424 - RNA, 20 nt, synthetic construct
  source 1..20: mol_type = other RNA
gagccaagcaaggcctaaca

SEQ ID NO: 425 - RNA, 20 nt, synthetic construct
  source 1..20: mol_type = other RNA
ggggagtctcagtcctgtgt

SEQ ID NO: 426 - RNA, 20 nt, synthetic construct
  source 1..20: mol_type = other RNA
gggcaggctcctctccaaaa

SEQ ID NO: 427 - RNA, 20 nt, synthetic construct
  source 1..20: mol_type = other RNA
gggggctccggcagcggacg

SEQ ID NO: 428 - RNA, 20 nt, synthetic construct
  source 1..20: mol_type = other RNA
gggcgtcgggcgaacaacgc

SEQ ID NO: 429 - RNA, 20 nt, synthetic construct
  source 1..20: mol_type = other RNA
gtgattgccgcagacatgct

SEQ ID NO: 430 - RNA, 20 nt, synthetic construct
  source 1..20: mol_type = other RNA
ggagggctgcgtcatgccgg

SEQ ID NO: 431 - RNA, 20 nt, synthetic construct
  source 1..20: mol_type = other RNA
cgcggcgctctaccacgatc

SEQ ID NO: 432 - RNA, 20 nt, synthetic construct
  source 1..20: mol_type = other RNA
tattggatatatcttcacgg

SEQ ID NO: 433 - RNA, 20 nt, synthetic construct
  source 1..20: mol_type = other RNA
ccccgggtgccggacgcatc

SEQ ID NO: 434 - RNA, 20 nt, synthetic construct
  source 1..20: mol_type = other RNA
acgtgactcaggaaagcacg

SEQ ID NO: 435 - RNA, 20 nt, synthetic construct
  source 1..20: mol_type = other RNA
atacccctctctcattaact

SEQ ID NO: 436 - RNA, 20 nt, synthetic construct
  source 1..20: mol_type = other RNA
agtgaagggtccagcagttc

SEQ ID NO: 437 - RNA, 20 nt, synthetic construct
  source 1..20: mol_type = other RNA
ggagaaccgggtacgcaacc

SEQ ID NO: 438 - RNA, 20 nt, synthetic construct
  source 1..20: mol_type = other RNA
aggcgtccgcaccacgagca

SEQ ID NO: 439 - RNA, 20 nt, synthetic construct
  source 1..20: mol_type = other RNA
ctgactccgcggcccccgac

SEQ ID NO: 440 - RNA, 20 nt, synthetic construct
  source 1..20: mol_type = other RNA
aggaaggcgctgcctcgcgt

SEQ ID NO: 441 - RNA, 20 nt, synthetic construct
  source 1..20: mol_type = other RNA
tgggtctggcacccagtatg

SEQ ID NO: 442 - RNA, 20 nt, synthetic construct
  source 1..20: mol_type = other RNA
ccagttcgcggtctgccatc

SEQ ID NO: 443 - RNA, 20 nt, synthetic construct
  source 1..20: mol_type = other RNA
gcggtggcaagcgaccgact

SEQ ID NO: 444 - RNA, 20 nt, synthetic construct
  source 1..20: mol_type = other RNA
ccgggatctagacggccgta